(19) United States
(12) Patent Application Publication
(10) Pub. No.: US 2008/0097558 A1
(43) Pub. Date: Apr. 24, 2008

(54) Method and apparatus for carrying out the controlled heating of dermis and vascular tissue

(75) Inventors: Philip E. Eggers, Dublin, OH (US); Andrew R. Eggers, Ostrander, OH (US); Eric A. Eggers, Portland, OR (US); Mark A. Mayerchak, Bothell, WA (US)

Correspondence Address: MUELLER AND SMITH, LPA;MUELLER-SMITH BUILDING, 7700 RIVERS EDGE DRIVE, COLUMBUS, OH, 43235, US

(73) Assignee: Apsara Medical Corporation, Columbus, OH (US)

(21) Appl. No.: 11/900,286

(22) Filed: Sep. 11, 2007

Related U.S. Application Data
(63) Continuation-in-part of application No. 11/825,902, filed on Jul. 10, 2007.
Application No. 11/825,902 is a continuation-in-part of application No. 11/583,621, filed on Oct. 19, 2006.

(51) Int. Cl. A61N 5/00

(52) U.S. Cl. 607/101

(57) ABSTRACT

Method, system and apparatus for carrying out a controlled heating of tissue in the region of skin dermis. A quasi-bipolar arrangement of wands or implants carrying multi-segmented active electrodes, temperature sensing and wand location LEDs as combined with a return electrode and heat sink function. Radiofrequency energization of the active electrodes is carried out in a ramping-up and pulsing fashion to provide relatively short therapy intervals. The combined return electrode and heat sink components may incorporate photo-detectors, which perform to insure proper alignment with the LED carrying active electrode sequences. An isotonic saline solution is located intermediate the contact surface of the combined return electrode and heat sink and the surface of skin over implanted active electrodes.

### CROSS-REFERENCE TO RELATED APPLICATIONS

This application is a continuation-in-part of copending U.S. patent application Ser. No. 11/825,902, filed Jul. 10, 2007, which, in turn, is continuation-in-part of copending U.S. application Ser. No. 11/583,621, filed Oct. 19, 2006.

### STATEMENT REGARDING FEDERALLY SPONSORED RESEARCH

Not applicable.

### BACKGROUND

The skin or integument is a major organ of the body present as a specialized boundary lamina, covering essentially the entire external surface of the body, except for the mucosal surfaces. It forms about 8% of the body mass with a thickness ranging from about 1.5 to about 4 mm. Structurally, the skin organ is complex and highly specialized as is evidenced by its ability to provide a barrier against microbial invasion and dehydration, regulate thermal exchange, act as a complex sensory surface, and provide for wound healing wherein the epidermis responds by regeneration and the underlying dermis responds by repair (inflammation, proliferation, and remodeling), among a variety of other essential functions.

Medical specialties have evolved with respect to the skin, classically in connection with restorative and aesthetic (plastic) surgery. Such latter endeavors typically involve human aging. The major features of the skin are essentially formed before birth and within the initial two to three decades of life are observed to not only expand in surface area but also in thickness. From about the third decade of life onward there is a gradual change in appearance and mechanical properties of the skin reflective of anatomical and biological changes related to natural aging processes of the body. Such changes include a thinning of the adipose tissue underlying the dermis, a decrease in the collagen content of the dermis, changes in the molecular collagen composition of the dermis, increases in the number of wrinkles, along with additional changes in skin composition. The dermis itself decreases in bulk, and wrinkling of senescent skin is almost entirely related to changes in the dermis. Importantly, age related changes in the number, diameter, and arrangement of collagen fibers are correlated with a decrease in the tensile strength of aging skin in the human body, and the extensibility and elasticity of skin decrease with age. Evidence indicates that intrinsically aged skin shows morphological changes that are similar in a number of features to skin aged by environmental factors, including photoaging.

See generally:
1. Gray's Anatomy, 39^{th} Edition, Churchill Livingstone, N.Y. (2005)
2. Rook's Textbook of Dermatology, 7^{th} Edition, Blackwell Science, Malden, Mass. (2004)

A substantial population of individuals seeking to ameliorate this aging process has evolved over the decades. For instance, beginning in the late 1980s researchers who had focused primarily on treating or curing disease began studying healthy skin and ways to improve it and as a consequence, a substantial industry has evolved. By reducing and inhibiting wrinkles and minimizing the effects of ptosis (skin laxity and sagging skin) caused by the natural aging of collagen fibrils within the dermis, facial improvements have been realized with the evolution of a broad variety of corrective approaches.

Considering its structure from a microscopic standpoint, the skin is composed of two primary layers, an outer epidermis which is a keratinized stratified squamous epithelium, and the supporting dermis which is highly vascularized and provides supporting functions. In the epidermis tissue there is a continuous and progressive replacement of cells, with a mitotic layer at the base replacing cells shed at the surface. Beneath the epidermis is the dermis, a moderately dense connective tissue. The epidermis and dermis are connected by a basement membrane or basal lamina with greater thickness formed as a collagen fiber which is considered a Type I collagen having an attribute of shrinking under certain chemical or heat influences. Lastly, the dermis resides generally over a layer of contour defining subcutaneous fat.

It has been widely recognized, beginning with Langer's 1861 report of lines of tension in cadaver skin, that aspects of the skin are under tension, and that the surface contour of skin is determined in part by mechanical forces. As demonstrated by Rose, et al., 1978 variations in the orientation of the fibrous network architecture of animal skin porcine model can affect dermal and hypodermal extensibility along particular force vectors. Utilizing a porcine skin model, Rose et al. demonstrated that the vectoral forces and orientations of Langer's lines (i.e. tension lines) vary in comparison between a pig with extensive adipose tissue and a leaner animal, with a lean animal having tension lines that are more transversely oriented relative to a heavier animal. It thus appears that the elastic fibers of the epithelial and subepithelial network are primarily oriented in parallel to the direction of the tension load.

Other research has demonstrated that similar tension lines are present in the skin tissue of other vertebrate animals, and that such tension lines of the pig are similar in mechanical character to those in human skin. For instance, Stell et al., 1980 demonstrated that the mechanical characteristics of pig skin are similar to the mechanical characteristics of human skin and show similar directional variation, with greater tension along the anterior-posterior axis of the animal than perpendicular to this axis. In humans, Langer's lines are not only representative of cleavage lines but also lines of tension and are indicative of the direction of minimal extensibility, i.e. greatest tension along a vector. While the amount of force necessary to stretch or reshape tissue varies in relation to the tissue orientation relative to tension lines, the terminal elastic modulus of skin tissue is the same for orientations parallel to tension lines as for orientations perpendicular to tension lines. In the pig, the ventral-caudal orientation appears to have the greatest laxity. For further background, see:
3. Rose, E. H., Vistnes, L. M., and Ksander, G. A., “A microarchitectural model of regional variations in hypodermal mobility in porcine and human skin.” Ann. Plastic Surg., 1: 252-266 (1978).
4. Stell, P. M., “The effects of varying degrees of tension on the viability of skin flaps in pigs.” Brit. J. Plastic Surg., 33: 371-376 (1980).

Early and some current approaches to the rejuvenation have looked to treatments directed principally to the epidermis, an approach generally referred to ablative resurfacing of the skin. Ablative resurfacing of the skin has been carried out with a variety of techniques. One approach, referred to as “dermabrasion” in effect mechanically grinds off components of the epidermis. Mechanical dermabrasion activities reach far back in history. It is reported that about 1500 B.C. Egyptian physicians used sandpaper to smooth scars. In 1905 a motorized dermabrasion was introduced. In 1953 powered dental equipment was modified to carry out dermabrasion practices. See generally:
5. Lawrence, et al., “History of Dermabrasion” Dermatol Surg, 26:95-101 (2000).

A corresponding chemical approach is referred to by dermatologists as “chemical peel”. See generally:
6. Moy, et al., “Comparison of the Effect of Various Chemical Peeling Agents in a Mini-Pig Model” Dermatol Surg. 22:429-432 (1996).

Another approach, referred to as “laser ablative resurfacing of skin” initially employed a pulsed CO₂ laser to repair photo-damaged tissue which removed the epidermis and caused residual thermal damage within the dermis. It is reported that patients typically experienced significant side effects following this ablative skin resurfacing treatment. Avoiding side effects, non-ablative dermal remodeling was developed wherein laser treatment was combined with timed superficial skin cooling to repair tissue defects related to photo-aging. Epidermal removal or damage thus was avoided, however, the techniques have been described as having limited efficacy. More recently, fractional photothermolysis has been introduced wherein a laser is employed to fire short, low energy bursts in a matrix pattern of non-continuous points to form a rastor-like pattern. This pattern is a formation of isolated non-continuous micro-thermal wounds creating necrotic zones surrounded by zones of viable tissue. See generally:
7. Manstein, et al., “Fractional Photothermolysis: A New Concept for Cutaneous Remodeling Using Microscopic Patterns of Thermal Injury” Lasers in Surgery and Medicine, 34:426-438 (2004).

These ablative techniques (some investigators consider fractional photothermolysis as a separate approach) are associated with drawbacks. For instance, the resultant insult to the skin may require 4-6 months or more of healing to evolve newer looking skin. That newer looking skin will not necessarily exhibit the same shade or coloration as its original counterpart. In general, there is no modification of the dermis in terms of a treatment for ptosis or skin laxity through collagen shrinkage.

To treat patients for skin laxity, some investigators have looked to procedures other than plastic surgery. Techniques for induced collagen shrinkage at the dermis have been developed. Such shrinkage qualities of collagen have been known and used for hundreds of years, the most classic example being the shrinking of heads by South American headhunters. Commencing in the early 1900s shrinking of collagen has been used as a quantitative measure of tanning with respect to leather and in the evaluation of glues. See:
8. Rasmussen, et al., “Isotonic and Isometric Thermal Contraction of Human Dermis I. Technic and Controlled Study”, J. Invest. Derm. 43:333-9 (1964).

Dermis has been heated through the epidermis utilizing laser technology as well as intense pulsed light exhibiting various light spectra or single wavelength. The procedure involves spraying a burst of coolant upon the skin such as refrigerated air, whereupon a burst of photons penetrates the epidermis and delivers energy into the dermis.

Treatment for skin laxity by causing a shrinkage of collagen within the dermis generally involves a heating of the dermis to a temperature of about 60° C. to 70° C. over a designed treatment interval. Heat induced shrinkage has been observed in a course of laser dermabrasion procedures. However, the resultant energy deposition within the epidermis has caused the surface of the skin to be ablated (i.e., burned off the surface of the underlying dermis) exposing the patient to painful recovery and extended healing periods which can be as long as 6-12 months. See the following publication:
9. Fitzpatrick, et al., “Collagen Tightening Induced by Carbon Dioxide Laser Versus Erbium: YAG Laser” Lasers in Surgery and Medicine 27: 395-403 (2000).

Dermal heating in consequence of the controlled application of energy in the form of light or radiofrequency electrical current through the epidermis and into the dermis has been introduced. To avoid injury to the epidermis, cooling methods have been employed to simultaneously cool the epidermis while transmitting energy through it. In general, these approaches have resulted in uncontrolled, non-uniform and often inadequate heating of the dermis layer resulting in either under-heating (insufficient collagen shrinkage) or over heating (thermal injury) to the subcutaneous fat layer and/or weakening of collagen fibrils due to over-shrinkage. See the following publication:
10. Fitzpatrick, et al., “Multicenter Study of Noninvasive Radiofrequency for Periorbital Tissue Tightening”, Lasers in Surgery in Medicine, 33:232-242 (2003).

The RF approach described in publication 8 above is further described in U.S. Pat. Nos. 6,241,753; 6,311,090; 6,381,498; and 6,405,090. Such procedure involves the use of an electrode capacitively coupled to the skin surface which causes radiofrequency current to flow through the skin in monopolar fashion to a much larger return electrode located remotely upon the skin surface of the patient. Note that the electrodes are positioned against skin surface and not beneath it. The radiofrequency current density caused to flow through the skin is selected to be sufficiently high to cause resistance heating within the tissue and reach temperatures sufficiently high to cause collagen shrinkage and thermal injury, the latter result stimulating beneficial growth of new collagen, a reaction generally referred to as “neocollagenesis”.

Uniform heating of the dermal layer generally is called for in the presence of an assurance that the underlying fat layer is not adversely affected while minimal injury to the epidermis is achieved. A discussion of the outcome and complications of the noted non-ablative mono-polar radiofrequency treatment is provided in the following publication:
11. Abraham, et al., “Current Concepts in Nonablative Radiofrequency Rejuvenation of the Lower Face and Neck” Facial Plastic Surgery, Vol. 21 No. 1 (2005).

In the late 1990s, Sulamanidze developed a mechanical technique for correcting skin laxity. With this approach one or more barbed non-resorbable sutures are threaded under the skin with an elongate needle. The result is retention of the skin in a contracted state and, over an interval of time, the adjacent tissue will ingrow around the sutures to stabilize the facial correction. See the following publications:
12. Sulamanidze, et al., “Removal of Facial Soft Tissue Ptosis With Special Threads”, Dermatol Surg., 28:367-371 (2002).
13. Lycka, et al., “The Emerging Technique of the Antiptosis Subdermal Suspension Thread”, Dermatol Surg., 30:41-44 (2004).

Eggers, et al., in application for U.S. patent Ser. No. 11/298,420 entitled “Aesthetic Thermal Sculpting of Skin”, filed Dec. 9, 2005 describes a technique for directly applying heat energy to dermis with one or more thermal implants providing controlled shrinkage thereof. Importantly, while this heating procedure is underway, the subcutaneous fat layer is protected by a polymeric thermal barrier. In one arrangement this barrier implant is thin and elongate and supports a flexible resistive heating circuit, the metal heating components of which are in thermal exchange contact with dermis. Temperature output of this resistive heating circuit is intermittently monitored and controlled by measurement of a monitor value of resistance. For instance, resistive heating is carried out for about a one hundred millisecond interval interspersed with one millisecond resistance measurement intervals. Treatment intervals experienced with this system and technique will appear to obtain significant collagen shrinkage within about ten minutes to about fifteen minutes. During the procedure, the epidermis is cooled by blown air.

Eggers et al., in application for U.S. patent Ser. No. 11/583,555 entitled “Method and Apparatus for Carrying Out the Controlled Heating of Tissue in the Region of Dermis”, filed Oct. 19, 2006 describes an improved utilization of such barrier implants wherein a slight pressure or tamponade is applied over the skin region during treatment to an extent effective to maintain substantially continuous conduction heat transfer between tissue in the region of the dermis and the implant heater segments. One result is an important lessening of required treatment time.

Eggers et al., in application U.S. patent Ser. No. 11/583,621 entitled “Method and Apparatus for Carrying Out the Controlled Heating of Tissue in the Region of Dermis”, filed Oct. 19, 2006 describes a bipolar radiofrequency implementation of the barrier implants wherein a continuous power modulating ramping up of power and electrode temperature occurs until a threshold level is reached. Once that level is reached, the continuous power is reduced for a soak interval. Treatment time is advantageously short with the bipolar R.F. approach.

Particularly where barrier implants are implemented using bipolar R.F. energy, protection of the epidermis from thermal damage has remained a concern. Cooling of the skin surface is called for at least during treatment. Such cooling must be sufficient to protect the epidermis while still permitting an effective heating of dermis to achieve proper collagen shrinkage.

Some of the procedures described above may be carried out using local anesthesia. Local anesthetic agents may be, for example, weakly basic tertiary amines, which are manufactured as chloride salts. The molecules are amphipathic and have the function of the agents and their pharmacokinetic behavior can be explained by the structure of the molecule. Such local anesthetics have a lipophilic side; a hydrophilic-ionic side; an intermediate chain, and, within the connecting chain, a bond. That bond determines the chemical classification of the agents into esters and amides. It also determines the pathway for metabolism. While there are a variety of techniques for administering local anesthesia, in general, it may be administered for infiltration, activity or as a nerve block. In each approach, the active anesthetic drug is administered for the purpose of intentionally interrupting neural function and thereby providing pain relief.

A variety of local anesthetics have been developed, the first agent for this purpose being cocaine, which was introduced at the end of the nineteenth century. Lidocaine is the first amide local anesthetic and the local anesthetic agent with the most versatility and thus popularity. It has intermediate potency, toxicity, onset, and duration, and it can be used for virtually any local anesthetic application. Because of its widespread use, more knowledge is available about metabolic pathways than any other agent. Similarly, toxicity is well known.

Vasoconstrictors have been employed with the local anesthetics. In this regard, epinephrine has been added to local anesthetic solutions for a variety of reasons throughout most of the twentieth century to alter the outcome of conduction blockade. Its use in conjunction with infiltration anesthesia consistently results in lower plasma levels of the agent. See generally:
14. “Clinical Pharmacology of Local Anesthetics” by Tetzlaff, J. E., Butterworth-Heinemann, Woburn, Mass. (2000).

To minimize the possibility of irreversible nerve injury in the course of using local anesthetics, the drugs necessarily are diluted. By way of example, the commonly used anesthetic drug is injected using concentrations typically in the range of 0.4% to 2.0% (weight percent). The diluent contains 0.9% sodium chloride. Such isotonic saline is used as the diluent due to the fact that its osmolarity at normal body temperature is 286 milliOsmols/liter which is close to that of cellular fluids and plasma which have a osmolarity of 310 milliOsmols/liter. As a result, the osmotic pressure developed across the semipermeable cell membranes is minimal when isotonic saline is injected. Consequently, there is no injury to the tissue's cells surrounded by this diluent since there is no significant gradient which can cause fluids to either enter or leave the cells surrounded by the diluent. It is generally accepted that diluents having an osmolarity in the range of 240 to 340 milliOsmols/liter are isotonic solutions and therefore can be safely injected.

A variety of aberrant vascular formations, i.e. angiomas, hemangiomas vascular malformations and other vascular anomalies, are present near the surface of the skin, such that these aberrant vascular formations display a visual or structural alteration of the appearance of the skin. Aberrant vascular formations may occur in arterial, venuous, or lymphatic tissues. Mulliken and Glowacki distinguished vascular anomalies (lesions) into two major categories, angiomas and vascular malformations. Vascular malformations are further subdivided and characterized as arterial, venuous, lymphatic, capillary and mixed (e.g., arterio-capillary-venuous). Jackson, et al., along with the ISSVA have provided further categorization of vascular lesions as being classified as either vascular tumors (i.e. angiomas, a term currently disfavored by the ISSVA, but utilized in the literature) or vascular malformations. See
15. Jackson, et al., “Hemangiomas, vascular malformations and lymphovenous malformations: classifications and methods of treatment.” Plat. Recon. Surg., 91: 1216-30 (1993).
16. “ISSVA Classification” (of vascular malformations) excerpt from Color Atlas of Vascular Tumors and Vascular Malformations, by O. Enjolras, M. Wassef and R. Chapot Cambridge University Press (2007).

Vascular tumors or angiomas are known as one type of aberrant vascular formation that are presented on the surface of the skin. Angiomas are an aberrantly or hyperplastically proliferating vascular tissue and include, for example, benign infantile hemangiomas; congenital hemangiomas; tufted angioma, with or without Kasabach-Merritt syndrome; Kaposiform hemangioendothelioma; spindle cell hemangioendothelioma; other rare hemangioendotheliomas, including epithelioid, composite, retiform, polymorphous, Dabska Tumor, and lymphangioendotheliomatosis; and dermatologic acquired vascular tumors, including pyogenic granuloma, targetoid hemangioma, glomeruloid hemangioma and microvenular heangioma. Hemangiomas are localized tumors of blood vessels, and may be generally classified, particularly with respect to infantile hemangiomas, as either proliferating (progressive growth), involuting (slowing rate of growth or regressing), or involuted (stable, with no further regression). Hemangiomas appear in approximately 10% of Caucasian infants, with complete regression occurring by age 7 in 70% of children. See:
17. Takahashi, et al., J. Clin. Invest. 93: 2357-2364. (June 1994).
   Angiomas exhibit increased endothelial cell turnover, and the proliferating stage is characterized by the expression of Type IV collagenase, and growth factors such as vascular endothelial growth factor (VEGF) and basic fibroblast growth factor (bFGF). Lymphangiomas are tumors of the lymphatic system and are usually benign and congenital, with approximately 75% occurring in the cervical region.

Jackson, et al., and the ISSVA classification subdivide a variety of nonproliferative vascular malformations, e.g., vascularity with quiescent endothelium and considered to be localized defects of vascular morphogenesis, and include such low flow vascular malformations such as, for instance, capillary malformations (CM), including Port-Wine stain (PWS), nevus flammeus, telangiectasia, and angiokeratoma; venuous malformation (VM), including, common sporadic VM, Bean syndrome, familial cutaneous and mucosal VM, glomuvenous malformation (GVM) and Mafucci syndrome; and lymphatic malformation (LM), fast-flow vascular malformations such as, for instance, arterial malformation, (AM); arteriovenous fistula (AVM) and arteriovenous malformation (AVM), and complex-combined vascular malformations such as, for instance CVM, CLM, LVM, CLVM, AVM-LM and CM-AVM.

Laser induced interstitial thermotherapy has been applied to the treatment of vascular anomalies, with effects differing from malignant cell necrosis, irreversible tissue damage or carbonization, depending on the maximum temperature to which the treated tissue has been heated. Successful outcomes utilizing tissue heating are highly dependent on effective monitoring of the temperature increase induced by localized heating, especially when vital anatomic structures are located in close proximity to treated tissues. Carbonization of tissue is difficult to completely avoid when utilizing laser induced interstitial thermotherapy, and once a carbonized tissue volume is created, this carbonized volume is not generally mobilized by metabolic processes, and can lead to deleterious side effects such as abscess formation. Thus, when utilizing thermotherapy techniques, reliable and accurate quantitative tissue temperature monitoring of great importance to avoid damage to healthy or untargeted tissue and organs, and to avoid induction of structures that might lead to deleterious side effects. One relatively unwieldy system available for tissue temperature monitoring is real-time magnetic resonance imaging. See, e.g.:
18. Eyrich et al., “Temperature mapping of magnetic resonance guided laser interstitial therapy (LITT) in lymphangiomas of the head and neck.” Lasers in Surgery and Medicine 26: 467-476 (2000).

Established treatment modalities for vascular anomalies include surgery, intralesional sclerotherapy and topical or interstitial heating using lasers, for instance Nd:YAG lasers. In most cases resection of extensive vascular anomalies in their entirety is not feasible, and unresected portions of a vascular anomaly may rapidly re-expand. Resection of a large lesions is hazardous due to risk of uncontrollable bleeding and mutilation of superficial surfaces due to extensive resection. Additional side effects of the above identified treatment modalities that suffer from inadequate control of tissue disruption include parethesia, tirsmus and local motoric plegia.

The dermis is the primary situs of congenital birthmarks generally deemed to be aberrant vascular formations or vascular lesions as capillary malformations, including those historically referred to as nevus flammeus and “Port-Wine Stains” (PWS). Ranging in coloration from pink to purple, these non-proliferative lesions are characterized histologically by ecstatic vessels of capillary or venular type within the papillary and reticular dermis and are considered as a type of vascular malformation. The macular lesions are relatively rare, occurring in about 0.3% of newborns and generally appear on the skin of the head and neck within the distribution of the trigeminal (fifth cranial) nerve. They persist throughout life and may become raised, nodular, or darken with age. Their depth has been measured utilizing pulsed photothermal radiometry (PPTR) and ranges from about 200 μm to greater than 1000 μm.

See the following publication:
19. Bincheng, et al., Accurate Measurement of Blood Vessel Depth in Port Wine Stain Human Skin in vivo Using “Photothermal Radiometry”, J. Biomed. Opt. (5), 961-966 (September/October 2004).

Fading or lightening the PWS lesions has been carried out with lasers with somewhat mixed results. For instance, they have been treated with pulsed dye lasers (PDL) at 585 mm wavelength with a 0.45 ms pulse length and 5 mm diameter spot size. Cryogenic bursts have been used with the pulsing for epidermal protection. Generally, the extent of lightening achieved is evaluated six to eight weeks following laser treatment. Such evaluation assigns the color of adjacent normal skin as 100% lightening and a post clearance, evaluation of lesions will consider more than 75% lightening as good.

See the following publication:
20. Fiskerstrand, et al., “Laser Treatment of Port Wine Stains: Therapeutic Outcome in Relation to Morphological Parameters” Brit. J. of Derm., 134, 1039-1043, (1996).

Capillary malformation lesions have been classified, for instance, utilizing video microscopy, three patterns of vascular ectasia being established; type 1, ectasia of the vertical loops of the papillary plexus; type 2, ectasia of the deeper, horizontal vessels in the papillary plexus; and type 3, mixed pattern with varying degrees of vertical and horizontal vascular ectasia. In general, due to the limited depth of laser therapy, only type 1 lesions are apt to respond to such therapy.

Port wine stains also are classified in accordance with their degree of vascular ectasia, four grades thereof being recognized, Grades I to IV. Grade 1 lesions are the earliest lesions and thus have the smallest vessels (50-80 um in diameter). Using ×6 magnification and transillumination, individual vessels can only just be discerned and appear like grains of sand. Clinically, these lesions are light or dark pink macules. Grade II lesions are more advanced (vessel diameter=80-120 um). Individual vessels are clearly visible to the naked eye, especially in less dense areas. They are thus clearly distinguishable macules. Grade III lesions are more ecstatic (120-150 um). By this stage, the space between the vessels has been replaced by the dilated vessels. Individual vessels may still be visible on the edges of the lesion or in a less dense lesion, but by and large individual vessels are no longer visible. The lesion is usually thick, purple, and palpable. Eventually dilated vessels will coalesce to form nodules, otherwise known as cobblestones. Grade IV represents the largest vessels. The main purpose of these classifications has been to assign a grade for ease in communication between practitioners and for ease of determination of the appropriate laser treatment settings.

See the following publication:
21. Mihm, Jr., et al, “Science, Math and Medicine—Working Together to Understand the Diagnosis, Classification and Treatment of Port-Wine Stains”, a paper presented in Mt. Tremblant, Quebec, Canada, 2004, Controversies and Conversations in Cutaneous Laser Surgery—An Advanced Symposium.

### SUMMARY

The present discourse is addressed to method, system and apparatus for carrying out the heating of tissue within the region of the dermis. An implant or wand carrying radiofrequency excitable active electrodes, associated electrode temperature sensors, and a thermal barrier functioning support is located at the interface between dermis and next adjacent subcutaneous tissue layer. The contact surface of a combined return electrode and heat sink is positioned against skin surface over the active electrode in combination with an electrically and thermally conductive material such as isotonic saline solution. Implanted active electrodes are then excited with radiofrequency energy. This excitation involves an initial power ramp-up to an active electrode setpoint threshold temperature, which may be elected for the therapy at hand, for instance, a cosmetic shrinkage of dermis or a treatment of vascular anomaly. When the setpoint threshold active electrode temperature is reached, essentially full power is applied to the electrodes in pulsed or intermittent fashion. This energization approach results in a short therapy interval. At the termination of the therapy interval, the heat sinking function is continued for a post therapy cool-down interval to avoid thermal injury to the epidermis.

To avoid thermal damage to the fat and muscle underlying the region of dermis, the radiofrequency pulse energization following powering a ramp-up energization interval may occur in relatively shorter time interval stages separated in time by cool-down inter-stages of time extent permitting the aforesaid underlying fat and muscle tissue to substantially return to normal body temperature. That inter-stage cool-down period may be pre-selected or established by a lower limit temperature level.

To improve performance, the wand or implant associated electrodes are configured in multi-segmented fashion. In one embodiment, the electrode segments are present in a longitudinally spaced-apart sequence, each arranged transversely to the implant axis. Such an arrangement promotes subsequent healing. In another embodiment, the electrode segments are present in a spaced-apart sequence, each arranged in parallel with the implant axis. This arrangement promotes thermal spreading.

The wands or implants carrying a sequence of electrodes, for example, four active electrodes, also may support two red region light emitting diodes aligned with and adjacent to the electrode sequence. Thus, when implanted within a heating channel, the location of the active electrode sequence is readily ascertained, by the practitioner.

The return electrode and heat sink apparatus may be configured to conform to facial contours. In this regard, the devices may be formed of a plurality of generally rectangular aluminum blocks having oppositely disposed slide surfaces, extending between an outer surface and a receiving surface. The blocks are joined in freely abutting relationship at the slide surfaces by a tensioned retainer located adjacent the outer surface and extending between oppositely disposed end blocks. A thin flexible thermally and electrically conductive shim extends across and is affixed to the sequence of blocks while permitting slideable movement to generally accommodate skin contours. A terminal assembly is configured to electrically couple the sequence blocks as a return electrode. That terminal assembly may also be configured to carry out a patient circuit safety monitoring function. The combined return electrode and heat sink apparatus also may be formed of a unitary aluminum block having a contact surface engageable with skin. In each embodiment however, there are provided two photo-detectors adjacent the contact surface, which provide a contact output when located over the emitting wand-carrying light emitting diodes. This assures a proper alignment of the device with the sequence of active electrodes. In case of loss of one of the contact outputs, a voice cue may be provided to the operator from a controller.

Where a sequence of four active electrodes are employed, they may be made of relatively shorter length, for example, 10 mm in length, space apart, for example, 1 or 2 mm. By positioning the sequence well forwardly on a given wand or implant, they may be repositioned within a common heating channel to permit the carrying out of two discrete therapies.

As another feature, a method is described for effecting a heating of tissue within the region of dermis of skin, which comprises the steps:

(a) determining a skin region for treatment;

(b) providing one or more implants each having one or more R.F. excitable active electrodes, each electrode being associated in thermal exchange relationship with a temperature sensor having a sensor output;

(c) providing one or more combined return electrode and heat sink assemblages each having one or more contact surfaces;

(d) determining one or more heating channel locations along the skin region, each extending from an entrance location;

(e) locating each implant along a heating channel generally at the interface between dermis and next adjacent subcutaneous tissue wherein the one or more active electrodes are contactable with dermis;

(e) selecting a setpoint temperature threshold level for the one or more electrodes;

(g) positioning said one or more combined return electrode and heat sink assemblages contact surfaces over and in substantial alignment with the active electrodes and against epidermis in heat and electrical transfer relationship and locating an electrical and thermally conductive material between the contact surface and the epidermis surface; and

(h) effecting a radiofrequency power energization at the one or more active electrodes for an energization interval wherein the energization is carried out for at least a portion of the energization interval during power-on intervals spaced apart in time by power-off intervals to reach and substantially maintain the setpoint temperature threshold level while avoiding thermal trauma to the epidermis.

Other objects of the disclosure will, in part, be obvious and will, in part, appear hereinafter.

The disclosure, accordingly, comprises the apparatus, method and system possessing the construction, combination of elements, arrangement of parts and steps, which are exemplified in the following detailed description.

For a fuller understanding of the nature and objects hereof, reference should had to the following detailed description taken in connection with the accompanying drawings.

### BRIEF DESCRIPTION OF THE DRAWINGS

FIG. 1 is a diagram of the structure of the extra-cellular matrix of dermis tissue;

FIG. 2 is a family of curves relating linear shrinkage of dermis of time and temperature;

FIG. 3 is a schema representing the organization of skin;

FIG. 4 is a perspective view of a wand or implant showing one version of a multi-segmented sequence of active electrodes;

FIG. 5 is a partial and enlarged view of the forward region of the implant of FIG. 4;

FIG. 6 is a perspective view of a wand or implant showing another active electrode segmented pattern;

FIG. 7 is an enlarged partial view of the forward region of the implant of FIG. 6;

FIG. 8 is an exploded view of the implant of FIG. 4;

FIG. 9 is a partial perspective view of the trailing end of an implant or wand showing resistor segment lead wrap-around features as well as a cable connector;

FIG. 10 is a bottom view of an implant with a portion broken away;

FIG. 11 is a side view of the implant of FIG. 10;

FIG. 12 is an enlarged side view of the trailing end of the implant of FIG. 11;

FIG. 13 is an enlarged partial top view of the implant of FIG. 4;

FIG. 14 is an enlarged view of the implant of FIG. 13 showing leads extending to the trailing end thereof;

FIG. 15 is an enlarged and broken away view of the implant of FIG. 6 illustrating electrode lead structure;

FIG. 16 is a further enlarged broken away view of the implant of FIG. 15 showing active electrode lead structure;

FIG. 17 is a top view of temperature sensing resistor segments of an implant or wand with portions broken away;

FIG. 18 is an enlarged view taken from the elliptical dashed region shown in FIG. 17;

FIG. 19 is an enlarged view taken within the rectangular dashed boundary shown in FIG. 17;

FIG. 20 is a broken away view showing in enlarged fashion the lead structure of the implant of FIG. 17;

FIG. 21 is a bottom view of an introducer instrument or needle;

FIG. 22 is a side view of the instrument of FIG. 21;

FIG. 23 is a schematic view showing epidermis, dermis, fat layer and underlying muscle in combination with an end view of an implant and combined return electrode and heat sink;

FIG. 24 is a perspective view of a multi-block combined return electrode and heat sink;

FIG. 25 is a sectional view taken through the plane 25-25 shown in FIG. 24;

FIG. 26 is schematic view of epidermis, dermis, fat layer and underlying muscle in combination with a side view of an implant showing two LEDs and four active electrodes and with a solid combined returned electrode and heat sink carrying two photodiodes and a thermocouple;

FIG. 27 is a schematic view showing the general system of the present discussion;

FIG. 28 is a block diagram of a controller employed with the system hereof;

FIG. 29 is a schematic view illustrating the performance of temperature sensor resistor segments, active electrodes and combined return electrode and heat sink;

FIG. 30 is a schematic representation of one active electrode energization scheme;

FIG. 31 is a top view of a component of an in vivo animal study;

FIG. 32 is a graph illustrating thermal dose limits for irreversible cell death of tissue;

FIG. 33 is a diagram illustrating temperatures and voltage levels versus time for a multi-stage pulse-mode method of active electrode energization;

FIGS. 34A-34H combine as labeled thereon to provide a flow chart of a method for carrying out cosmetic dermis contraction;

FIG. 35 is a schematic representation of a human head showing the location of an implant at the dermis-next subcutaneous tissue layer interface within a heating channel location in parallel with relaxed intrinsic skin tension lines; and

FIGS. 36A-36H combine as labeled thereon to provide a flow chart describing a method for thermally treating a vascular anomaly.

### DETAILED DESCRIPTION OF THE INVENTION

The arrangement of the physical structure of the dermis is derived in large part from the structure of the extracellular matrix surrounding the cells of the dermis. The term extracellular matrix (ECM) refers collectively to those components of a tissue such as the dermis that lie outside the plasma membranes of living cells, and it comprises an interconnected system of insoluble protein fibers, cross-linking adhesive glycoproteins and soluble complexes of carbohydrates and carbohydrates covalently linked to proteins (e.g. proteoglycans). A basement membrane lies at the boundary of the dermis and epidermis, and is structurally linked to the extracellular matrix of the dermis and underlying hypodermis. Thus the extracellular matrix of the dermis distributes mechanical forces from the epidermis and dermis to the underlying tissue.

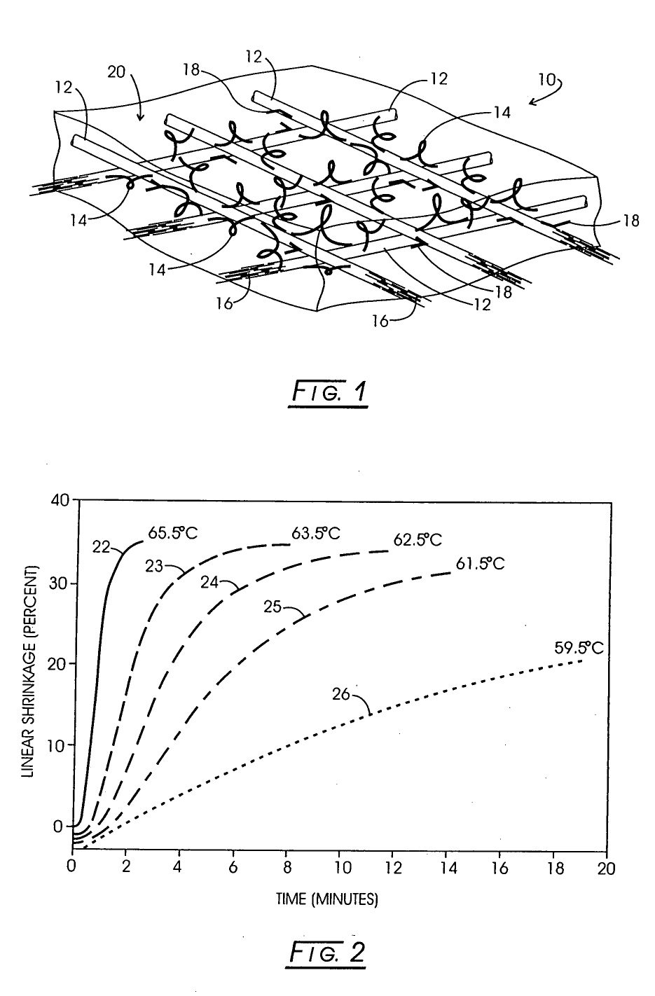

Looking to FIG. 1, a schematic representation of a region of the extracellular matrix of the dermis is represented generally at 10. The insoluble fibers include collagen fibers at 12, most commonly collagen Type I, and elastin at 14. The fundamental structural unit of collagen is a long, thin protein (300 nm×15 nm) composed of three subunits coiled around one another to form the characteristic right-handed collagen triple helix. Collagen is formed within the cell as procollagen, wherein the three subunits are covalently cross-linked to one another by disulphide bonds, and upon secretion are further processed into tropocollagen. The basic tropocollagen structure consists of three polypeptide chains coiled around each other in which the individual collagen molecules are held in an extended conformation. The extended conformation of a tropocollagen molecule is maintained by molecular forces including hydrogen bonds, ionic interactions, hydrophobicity, salt links and covalent cross-links. Tropocollagen molecules are assembled in a parallel staggered orientation into collagen fibrils at 16, each containing a large number of tropocollagens, held in relative position by the above listed molecular forces and by cross-links between hydrolysine residues of overlapping tropocollagen molecules. Certain aspects of collagen stabilization are enzyme mediated, for example by Cu-dependent lysyl oxidase. Collagen fibrils are typically of about 50 nm in diameter. Type I collagen fibrils have substantial tensile strength, greater on a weight basis than that of steel, such that the collagen fibril can be stretched without breaking. Collagen fibrils are further aggregated into more massive collagen fibers, as previously shown at 12. The aggregation of collagen fibers involves a variety of molecular interactions, such that it appears that collagen fibers may vary in density based on the particular interactions present when formed. Elastin, in contrast to collagen, does not form such massive aggregated fibers, may be thought of as adopting a looping conformation (as shown at 14) and stretch more easily with nearly perfect recoil after stretching.

The extracellular matrix (ECM) as at 10 lies outside the plasma membrane, between the cells forming skin tissue. ECM components including tropocollagen, are primarily synthesized inside the cells and then secreted into the ECM through the plasma membrane. The overall structure and anatomy of the skin, and in particular the dermis, are determined by the close interaction between the cells and ECM. Referring again to FIG. 1, only a few of the many and diverse components of the ECM are shown. In addition to collagen fibers 12 and elastin 14 are a large number of other components that serve to crosslink or cement these named components to themselves and to other components of the ECM. Such crosslinking components are represented as at 18, and may be of protein, glycoprotein and or carbohydrate composition, for example. The cross-linked collagen fibers shown in FIG. 1 are embedded in a layer of highly hydrated material, including a diverse variety of modified carbohydrates, including particularly the large carbohydrate hyaluronic acid (hyaluronan) and chondroitin sulphate. Hyaluronan is a very large, hydrated, non-sulphated mucopolysaccaride that forms highly viscous fluids. Chondroitin sulphate is a glycosaminoglycan component of the ECM. Accordingly, the volume of the ECM as represented generally at 20 is filled with a flexible gel with a hydrated hyaluronan component that surrounds and supports the other structural components such as collagen and elastin. Thus the structural form of the dermis may be thought to be composed of collagen, providing tensile strength, with the collagen being held in place within a matrix of hyaluronan, which resists compression. Underlying this structure are the living cells of the dermis, which in response to stimuli (such as wounds or stress, for instance) can be induced to secrete additional components, synthesize new collagen (i.e. neocollagenesis), and otherwise alter the structural form of the ECM and the skin itself. The structure of the collagen reinforced connective tissues should not be considered entirely static, but rather that the net accumulation of collagen connective tissues is an equilibrium between synthesis and degradation of the components of the collagen reinforced connective tissues. Similarly, the other components of the ECM are modulated in response to environmental stimuli. In essence, the ultrastructural characteristics of the dermis, combining cellular and extracellular elements, including collagen fibers, determine the mechanical behavior of the dermis and provide the predominate determinants of the mechanical behavior of skin. Recognizing that collagen fibers are one of the primary load bearing structures of the skin, and such fibers possessing significant tensile strength, certain load bearing collagen fibers are primarily oriented in parallel to the direction of the tension load on the skin structure. Additional disclosure relevant to the mechanobiology of force transduction in skin may be found be referring to:
22. Silver et al., Skin Research and Technology, 9: 3-23 (2003).

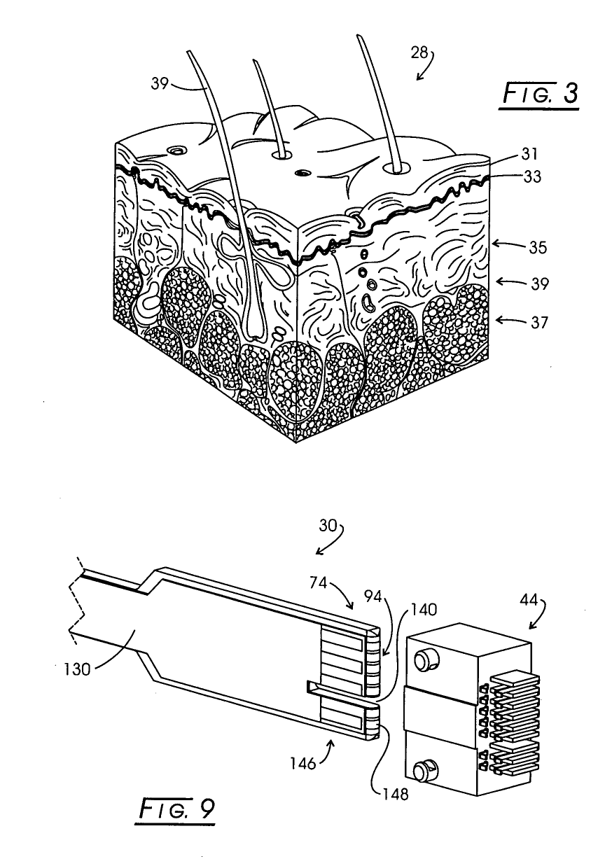

As noted earlier previous researchers have shown that collagen fibers can be induced to shrink in overall length by application of heat. Experimental studies have reported that collagen shrinkage is, in fact, dependent upon the thermal dose (i.e., combination of time and temperature) in a quantifiable manner. (See publication 16, infra). Looking to FIG. 2, a plot of linear collagen shrinkage versus time for various constant temperatures is revealed in association with plots or lines 22-26. For instance, at line 24, linear shrinkage is seen to be about 30% for a temperature of 62.5° C. held for a ten minute duration. Curve 24 may be compared with curve 22 where shrinkage of about 36% is achieved in very short order where the temperature is retained at 65.5° C. Correspondingly, curve 26 shows a temperature of 59.5° C. and a very slow rate of shrinkage, higher levels thereof not being reached. Clinicians generally would prefer a shrinkage level on the order of 10% to 20% in dealing with skin laxity.

FIG. 3 reveals a schema representing the organization of skin. Shown generally at 28, the illustrated skin structure is one of two major skin classes of structure and functional properties representing thin, hairy (hirsute) skin which constitutes the great majority of the body's covering. This is as opposed to thick hairless (glabrous) skin from the surfaces of palms of hands, soles of feet and the like. In the figure, the outer epidermis layer 31 is shown generally having inwardly disposed rete ridges or pegs 33 and extending over the dermis layer represented generally at 35. Dermis 35, in turn, completes the integument and is situated over an adjacent subcutaneous tissue layer represented generally at 37. Those involved in the instant subject matter typically refer to this adjacent subcutaneous layer 37 which has a substantial adipose tissue component as a “fat layer” or “fatty layer,” and this next adjacent subcutaneous tissue layer is also called the “hypodermis” by some artisans. The figure also reveals a hair follicle and an associated shaft of hair 39. Not shown in FIG. 3 are a number of other components, including the cellular structure of the dermis, and the vascular tissues supplying the vascularized dermis and its overlying epidermis.

Epidermis 31 in general comprises an outer or surface layer, the stratum corneum, composed of flattened, cornified non-nucleated cells. This surface layer overlays a granular layer, stratum granulosum, composed of flattened granular cells which, in turn, overlays a spinous layer, stratum spinosum, composed of flattened polyhedral cells with short processes or spines and, finally, a basal layer, stratum basale, composed columnar cells arranged perpendicularly. For the type skin 28, the epidermis will exhibit a thickness from about 0.07 to 0.20 mm. Heating implants or wands described herein will be seen to be contactable with the dermis 35 at a location shown generally at 39 representing the interface between dermis 35 and next adjacent subcutaneous tissue or fat layer 37. The dermis in general comprises a papillary layer, subadjacent to the epidermis, and supplying mechanical support and metabolic maintenance of the overlying epidermis. The papillary layer of the dermis is shaped into a number of papillae that interdigitate with the basal layer of the epidermis, with the cells being densely interwoven with collagen fibers. The reticular layer of the dermis merges from the papillary layer, and possesses bundles of interlacing collagen fibers (as shown in FIG. 1) that are typically thicker than those in the papillary layer, forming a strong, deformable, three dimensional lattice around the cells of the reticular dermis. Generally, the dermis is highly vascularized, especially as compared to the avascular epidermis. The dermis layer 34 will exhibit a thickness of from about 1.0 mm to about 3.0 mm to 4.0 mm.

For the purposes of the application, “intradermal” is defined as within the dermis layer of the skin itself. “Subcutaneous” has the common definition of being below the skin, i.e. near, but below the epidermis and dermis layers. “Subdermal” is defined as a location immediately interior to, or below the dermis, at the interface 40 between the dermis and the next adjacent subcutaneous layer. “Hypodermal” is defined literally as under the skin, and refers to an area of the body below the dermis, within the hypodermis, and is usually not considered to include the subadjacent muscle tissue. “Peridermal” is defined as in the general area of the dermis, whether intradermal, subdermal or hypodermal. Transdermal is defined in the art as “entering through the dermis or skin, as in administration of a drug applied to the skin in ointment or patch form,” i.e. transcutaneous. A topical administration as used herein is given its typical meaning of application at skin surface.

As noted, the thickness of the epidermis and dermis vary within a range of only a few millimeters. Thus subcutaneous adipose tissue is responsible in large part for the overall contours of the skin surface, and the appearance of the individual patient's facial features, for instance. The size of the adipose cells may vary substantially, depending on the amount of fat stored within the cells, and the volume of the adipose tissue of the hypodermis is a function of cell size rather than the number of cells. The cells of the subcutaneous adipose tissue, however, have only limited regenerative capability, such that once killed or removed, these cells are not typically replaced. Any treatment modality seeking to employ heat to shrink the collagen of the ECM of the skin, must account for the risk associated with damaging or destroying the subcutaneous adipose layer, with any such damage representing a large risk of negative aesthetic effects on the facial features of a patient.

In general, the structural features of the dermis are determined by a matrix of collagen fibers forming what is sometimes referred to as a “scaffold.” This scaffold, or matrix plays an important role in the treatment of skin laxity in that once shrunk, it must retain it's position or tensile strength long enough for new collagen evolved in the healing process to infiltrate the matrix. As noted above, that process is referred to as “neocollagenesis.” Immediately after the collagen scaffold is heated and shrunk portions of it are no longer vital because of having been exposed to a temperature evoking an irreversible denaturation. Where the scaffold retains adequate structural integrity in opposition to forces that would tend to pull it back to its original shape, a healing process requiring about four months will advantageously occur. During this period of time, neocollagenesis is occurring, along with the deposition and cross linking of a variety of other components of the ECM. In certain situations, collagen is susceptible to degradation by collagenase, whether native or exogenous.

Studies have been carried out wherein the mechanical properties of collagen as heated were measured as a function of the amount of shrinkage induced. The results of one study indicated that when the amount of linear shrinkage exceeds about 20%, the tensile strength of the collagen matrix or scaffold is reduced to a level that the contraction may not be maintained in the presence of other natural restorative forces present in tissue. Hence, with excessive shrinkage, the weakened collagen fibrils return from their now temporary contracted state to their original extended state, thereby eliminating any aesthetic benefit of attempted collagen shrinkage. The current opinion of some investigators is that shrinkage should not exceed about 25%.

One publication reporting upon such studies describes a seven-parameter logistic equation (sigmoidal function) modeling experimental data for shrinkage, S, in percent as a function of time, t, in minutes and temperature, T, in degrees centigrade. That equation may be expressed as follows:

$$S ( t , T ) = \frac{[ {a}_{0} ( T - 62 ) + {a}_{1} ] - {a}_{2}}{1 + {( \frac{t}{{a}_{3} {e}^{- a [ T - 62 ]}} )}^{( {a}_{4} ( T - 62 ) + {a}_{5} )}} + {a}_{2} \qquad ( 1 )$$

where:
a₀, a₁, a₂, a₃, a₄, and a₅, are empirically derived constant coefficients.
T is the temperature in degrees Centigrade
t is the exposure duration in minutes
S is the level of shrinkage or contraction in percent.

Equation (1) may, for instance, be utilized to carry out a parametric analysis relating treatment time and temperature with respect to preordained percentages of shrinkage. For example, where shrinkage cannot be observed by the clinician then a time interval of therapy may be computed on a preliminary basis. For further discourse with respect to collagen matrix shrinkage, temperature and treatment time, reference is made to the following publication:
23. Wall, et al., “Thermal Modification of Collagen” Journal of Shoulder and Elbow Surgery, 8:339-344 (1999).

When practicing the embodiments as disclosed herein, it will be useful to deliver the increase of tension due to heat induced shrinkage parallel to the tension lines of the face, thus enhancing the reduction of skin laxity. As utilized herein, Langer's lines are analogous to vectoral lines of increased skin tension, i.e. skin tension lines. In contrast are generally perpendicular zones of relaxed skin or “relaxed intrinsic skin tissue lines”. As an example of how the use of the present embodiments can be enhanced, in one experiment known in the art, full thickness specimens of human cadaver vital abdominal skin, including subcutaneous fat, skin was heated parallel to Langer's lines. These specimens were exposed to immersion in water at temperatures ranging from 37° C. to 97° C. for 1 minute. In these experiments, a maximum of about 40-45% shrinkage was reported compared to shrinkage of about 25 to 30% perpendicular to Langer's lines. In these specimens, a 59-62° C. threshold temperature for shrinkage was noted with no additional shrinkage at temperatures higher than 70° C. Indications of cell death were first noted at 56° C., with such indications including denaturation of fat. In these experiments, deep reticular dermis demonstrated greater amounts of shrinkage than papillary dermis, while minimal to no epidermal shrinkage was noted.

Additional testing in human cadaver abdominal skin demonstrated the effect of Langer's lines or tension lines on the orientation of shrinkage. Specimens of human tissue had an oval hole punched in the middle, with heating at 80° C. for 1 minute resulting in shrinkage along the modified Langer's lines, parallel to the Langer's lines (of a maximum 47%) enhanced over shrinkage perpendicular to these Langer's lines (of a maximum 29%). These experiments demonstrate, that when utilizing the apparatus and method disclosed herein, which produce controlled heating along a linear path, that cognizance of the predicted location of Langer's lines will be useful in guiding the practitioner in utilizing the apparatus and method to effectively sculpt the body surface contour through heat induced shrinkage. Placement of wands may be oriented in a manner to take advantage of increased shrinkage parallel to relaxed intrinsic skin tissue lines and relatively reduced shrinkage perpendicular or oblique to relaxed intrinsic skin tissue lines. Application of the apparatus and method is preferably utilized by accounting for a predicted increased relative linear shrinkage of collagen fibers, wherein such fibers are preferentially oriented along skin tension lines. Experience on the part of the practitioner will provide guidance as to the location of tensional vectors, along which maximal shrinkage would be expected to occur. In addition, a variety of techniques are known that will be useful in assisting with locating the vectors of tension lines. Non-methods are available for identifying the preferential orientation of collagen fibers, and allowing prediction of the location of skin tension lines. See further discourse in relation to FIG. 35 herein, a face detail of Langer's lines from Netter's Clinical Anatomy. For additional disclosure relative to identifying the location of relaxed intrinsic skin tissue lines see:
24. Nickell, S., Hermann, M., Essenpreis, M., Farrell, T. J., Kramer, U., and Patterson, M. S., “Anisotropy of light propagation in human skin.” Phys. Med. Biol., 45: 2873-2886 (2000).

With the present treatment approach, dermis is heated by radiofrequency current passing from four active electrodes located on a wand and positioned at the interface between dermis and the next subcutaneous tissue or fat layer. These four active electrodes are associated in quasi bipolar relationship with the contact surface of a metal combined heat sink and return electrode. Coupling at the epidermis surface between the return electrode and the active electrodes is through a thermally and electrically conductive medium which preferably is isotonic saline solution. To protect the subcutaneous layer, the four electrodes are supported upon a polymeric thermal barrier. That barrier support is formed of a polymeric resin such as polyetherimide available under the trade designation “Ultem” from the plastics division of General Electric Company of Pittsfield, Mass.

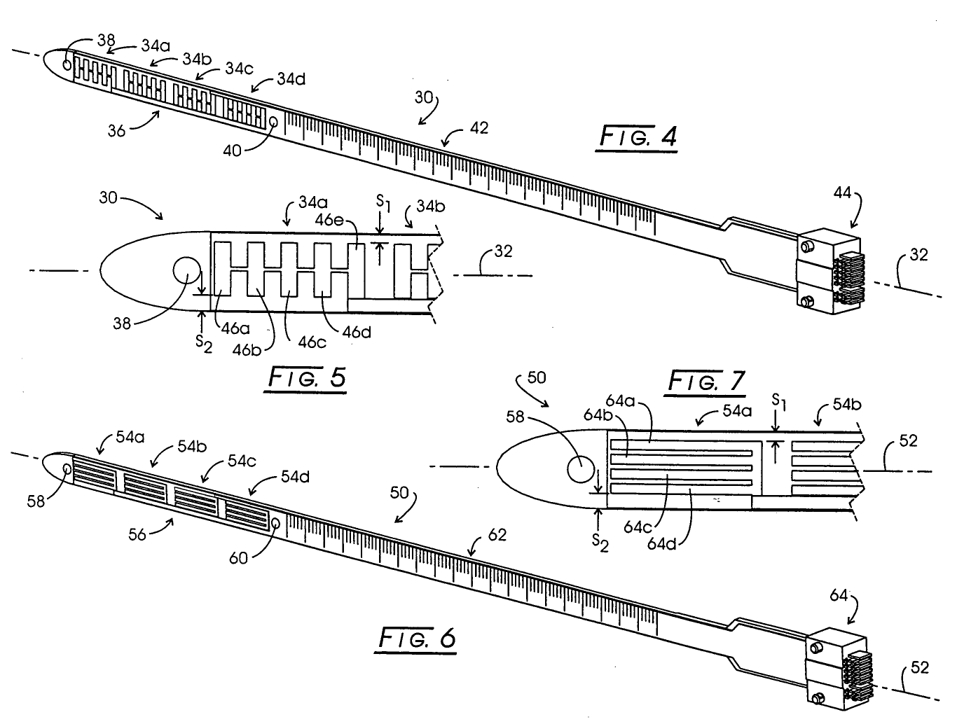

Looking to FIG. 4, one embodiment of the wand structure according to the invention is represented in general at 30. Wand 30 has an overall length along its longitudinal axis 32 of about 7.7 inches and a widthwise dimension along an insertion length of about 5 mm. Its four electrodes as seen at 34a-34d have lengths along axis 32 of 10 mm and are mutually spaced apart 1 mm such that electrode region represented in general at 36 has an overall length of about 43 mm. The effective width of these electrodes is about 3.7 mm to about 3.8 mm. Aligned with electrodes 34a-34d as well as wand axis 32 is a distal light emitting diode (LED) 38. In similar fashion, a light emitting diode 40 is located proximal to the electrode array at region 36 and in alignment therewith as well as axis 32. LEDs 38 and 40 are energizable to emit light in a region of the spectrum effective to penetrate tissue, for example, in the red region. Note that the electrode region 36 is located well forwardly upon the wand 30. Positioned behind region 36 are wand positioning indicia represented generally at 42. Indicia 42 aid the clinician in positioning region 36 within a heating channel by observing the indicia with respect to an entrance incision to such heating channel. Of course, when energized, LEDs 38 and 40 also permit accurate positioning. At the rearward end of wand 30 is a commercially available cable connector represented generally at 44. Connector 44 may be provided, for example, as a type MECI-108-02-X-D-RAI-SL, marketed by Samtec, Inc. of New Albany Ind. With that connector as at 44, over and under contacts are provided which are in mutual alignment.

Looking additionally to FIG. 5, note that each of the electrodes 34a-34d are formed with five electrically interconnected electrode segments which are identified in FIG. 5 at 46a-46e. These electrode segments are present in a longitudinally spaced apart sequence, each arranged transversely to the implant axis 32. Such segments generally are employed because solid, peripherally rectangular electrodes tend to overheat the dermis region of interest. By utilizing the transverse arrangement shown in FIGS. 4 and 5, zones of heating are created during use and subsequent to a given procedure, such zone heating will promote healing.

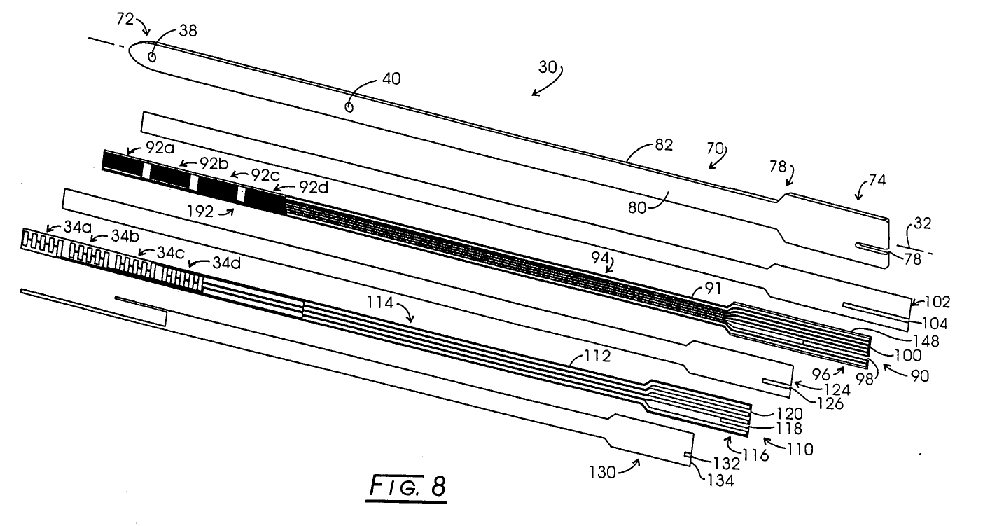

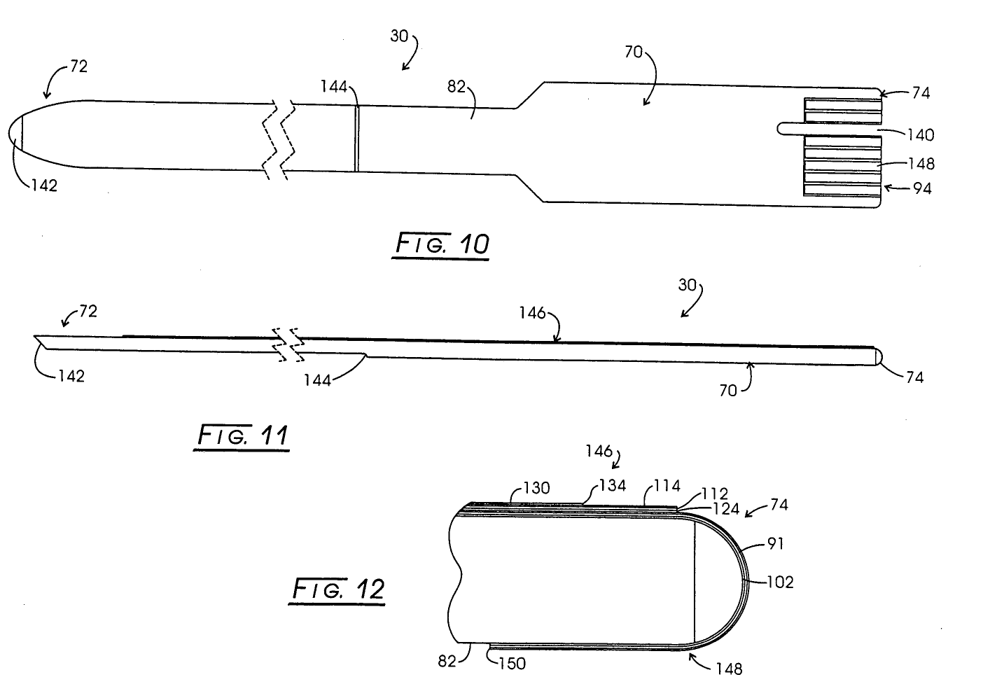

Looking to FIG. 6, another wand embodiment is disclosed in general at 50. Wand 50 is symmetrically disposed about a longitudinal axis or wand axis 52 and, as before, is formed with four active electrodes 54a-54d symmetrically disposed about wand axis 52. Wand 50 has an insertion length with a width of about 5 mm, while electrodes 54a-54d have an overall width of about 3.7 mm-3.8 mm and lengths of 10 mm. These electrodes are spaced apart about 1 mm such that the electrode region 56 has a length along axis 52 of about 43 mm. Inasmuch as the wand 50 will have an overall length of about 7.7 inches, region 56 is spaced well forwardly. As is the case of implant or wand 30, implant or wand 50 incorporates a distal light emitting diode 58 configured in the same manner as LED 38. LED 58 is aligned with wand axis 52 as well as active electrodes 54a-54d. In similar fashion, a proximal LED light emitting diode 60 will be located rearwardly of region 56, in alignment with wand axis 52 as well as electrodes 54a-54d. LEDs 58 and 60 are energizable to emit light in a region of the spectrum effective to penetrate tissue, for example, the red region. Extending rearwardly from region 56 are visible indicia represented generally at 62. Finally, at the trailing end of the implant or wand 50 is a cable connector represented generally at 64. Connector 64 may be identical to that described at 44 in FIG. 4.

Looking additionally to FIG. 7, it may be observed that electrodes 54a-54d are formed with four electrically interconnected electrode segments. These segments are identified at 64a-64d in FIG. 7 with respect to electrode 54a. Electrode segments as at 64a-64d are present in a spaced apart sequence, each arranged in parallel with implant axis 52. Such a parallel pattern avoids the above-noted overheating phenomena and provides the advantage of evoking a thermal spreading effect. While such an arrangement does not promote healing as described in connection with FIGS. 4 and 5, the transverse segment arrangement of those figures does not promote thermal spreading.

In the course of carrying out in vivo animal experiments with wands as at 30 and 50 it was determined that the set back distances s₁ and S₂, as illustrated in FIGS. 5 and 7 were quite important in avoidance of aberrant currents extending to subcutaneous fat and muscle. This was particularly true where the fat layer was less than about 2.5 mm in thickness. Note that these set backs extend between the widthwise edges of electrode 34a-34d, and 54a-54d and the adjacent edges of the thermal barrier and support described in FIG. 8 at 70. Set backs s₁ and S₂, should fall within a range from about 0.030 inch to about 0.100 inch. Preferably a minimum such set back is 0.040 inch.

Referring to FIG. 8, an exploded view of thermal wand or implant 30 is again represented in general with that numeration. Implant 30 incorporates a support and thermal barrier represented generally at 70. Barrier 70 is formed of the earlier-described polyetherimide and extends along a longitudinal wand axis 32 between a leading end represented in general at 72 and a trailing end represented generally at 74. Support 70 exhibits an arbitrarily designated insertion length extending from leading end 72 to about position 78. In addition to supporting LEDs 38 and 40, in general, the width of support 70 along the so called insertion length will be about 5 mm and its thickness adjacent trailing end 74 will be about 0.054 inch. Note that a slot 78 extends inwardly from trailing end 74. This slot is a slot component for purposes of attaining proper registration with the earlier-described connectors as at 44 or 64 discussed in connection with respective FIGS. 4 and 6. Generally, the device 70 will have an arbitrarily designated support surface represented at 80 as well as an arbitrarily designated oppositely disposed insulative surface 82. Initially positioned over support surface 80 is a temperature sensing resistor carrying flexible circuit or flex circuit represented generally at 90. Circuit 90 is configured with a thin (0.001 inch) polyimide (Kapton) or flexible substrate 91 which, in turn, carries four temperature sensing resistor segments 92a-92d from which extend an initial sequence of leads represented generally at 90 and which extend to an enlarged rearward lead assemblage represented in general at 96. Note the presence of a slot 98 which, as before participates in proper registration with cable connectors. It may be noted that the trailing end 100 of lead assemblage 74 extends further outwardly rearwardly from corresponding trailing end 74 of support 70. This is because it will be bent around that trailing end 74 during assembly to provide temperature sensing resistor contacts for engagement with a cable connector. Flex circuit 90 is secured to support surface 88 of support 70 by a medical grade pressure sensitive adhesive represented generally at 102. Resistor segments 92a-92d and their related lead structuring are formed of % ounce copper having a thickness of about 0.00035 inch. Segments 92a-92d are configured with trace widths of 0.003 inch and spacing between trace lengths of that same width. This permits development of a 10-15 ohm resistance measurement. Such copper thickness also permits the bending of the rearward portion of the lead structure over trailing end 74 of support 70. Note the cable connector registration participating slot 104 of implant or thermal wand 50 (FIG. 6) is configured in the same manner with the same dimensions to this point in the assembly. However their electrode circuits differ but only in terms of their electrode patterns.

In FIG. 8, the electrode flex circuit supported from support surface 80 is represented in general at 110. Circuit 110 is formed with a thin polyimide (Kapton) substrate or support 112 having a thickness of 0.001 inch which in turn, supports four electrodes shown as transversely segmented and identified with the same numeration as FIG. 4 at 34a-34d. From electrodes 34a-34d extend lead traces represented in general at 114 which, in turn, extend to a lead contact region of expanded width represented generally at 116. Note the slot 118 extending inwardly from the circuits' trailing end 120. As before, this slot is for the purpose of contributing to registration in connection with cable connectors as at 44 described in connection with FIG. 4. Flex circuit 110 is adhesively attached to the outward face of flex circuit 90 by a pressure sensitive adhesive as represented in general at 124. Note the registration slot 126. The electrodes and leads of the flex circuits of implants 30 and 50 are formed of gold plated copper with a thickness of about 0.0014 inch.

The uppermost layer of implant 30 is a coverlay represented in general at 130. Coverlay 130 is electrically insulative and covers all otherwise exposed leads extending from electrodes 34a-34d. Note that it also defines a slot 132 and has a trailing end or termination 134 permitting the lead contact region 116 to make an appropriate electrical contact with the corresponding contact members of cable connectors as at 44 (FIG. 4).

Looking momentarily to FIG. 9, a trailing end region of implant or wand 30 is represented along with one of the noted cable connectors as described at 44 in FIG. 4. Note that coverlay 130 terminates before trailing end 74 is reached and that the seven lead array described in conjunction with temperature sensing resistor circuit 90 has been wrapped around to the bottom side of the implant. Accordingly, the upper and lower contacts of cable connector 44 may be electrically associated with both of the flex circuits 90 and 110. Note the compiled registration slot 140.

Turning now to FIG. 10, a bottom view of the implant 30 is represented showing the insulated surface of support 70 and its trailing end 74. Note that seven resistor segment leads are revealed in their wrapped around orientation again identified at 94 and slot 140 is seen to reappear. Looking additionally to FIG. 11, the leading end 72 of wand 30 is seen to be formed in the shape of a half ellipse and is slanted upwardly and outwardly at 142 to evoke what may be considered a “sled” effect biasing the support 70 toward dermis inward side. Note, additionally that the thickness is stepped down at a radiused position 144. FIG. 11 reveals the assemblage of two flex circuits as represented in general at 146.

FIG. 12 is a partial side view of trailing end 74, showing the two flex circuit assemblage represented generally at 146 and the enlarged lead wrap around portion 148. Components described in conjunction with FIG. 8 again are identified with the same numeration.

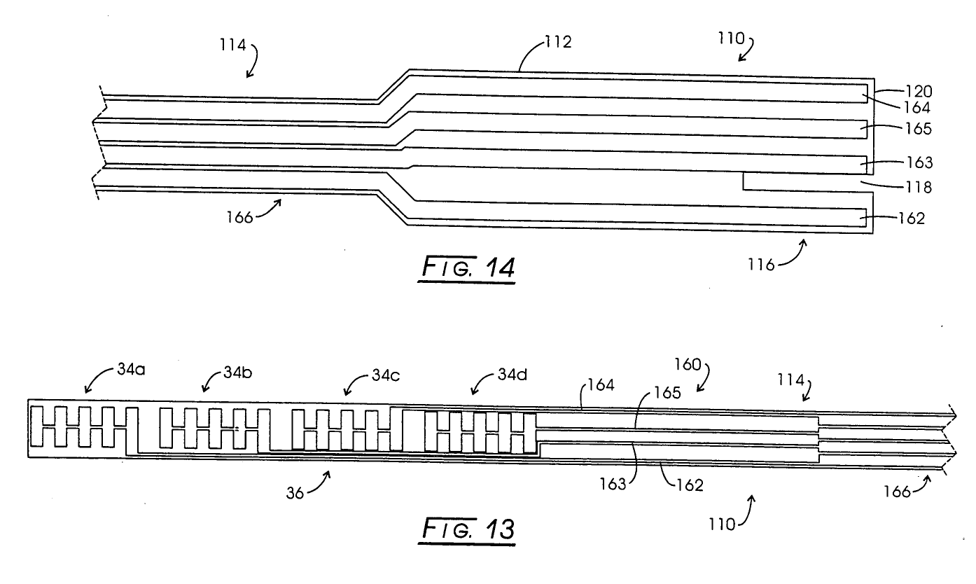

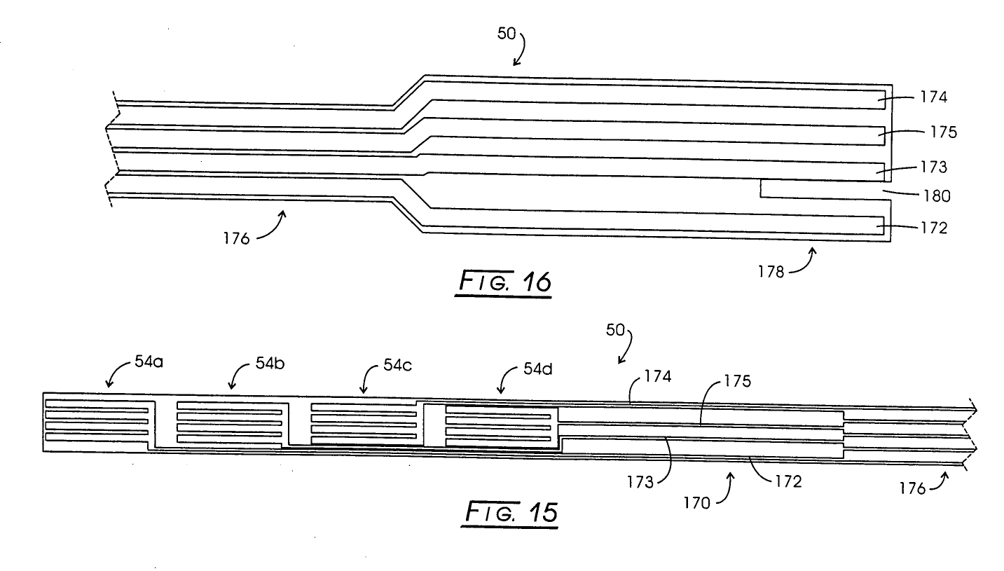

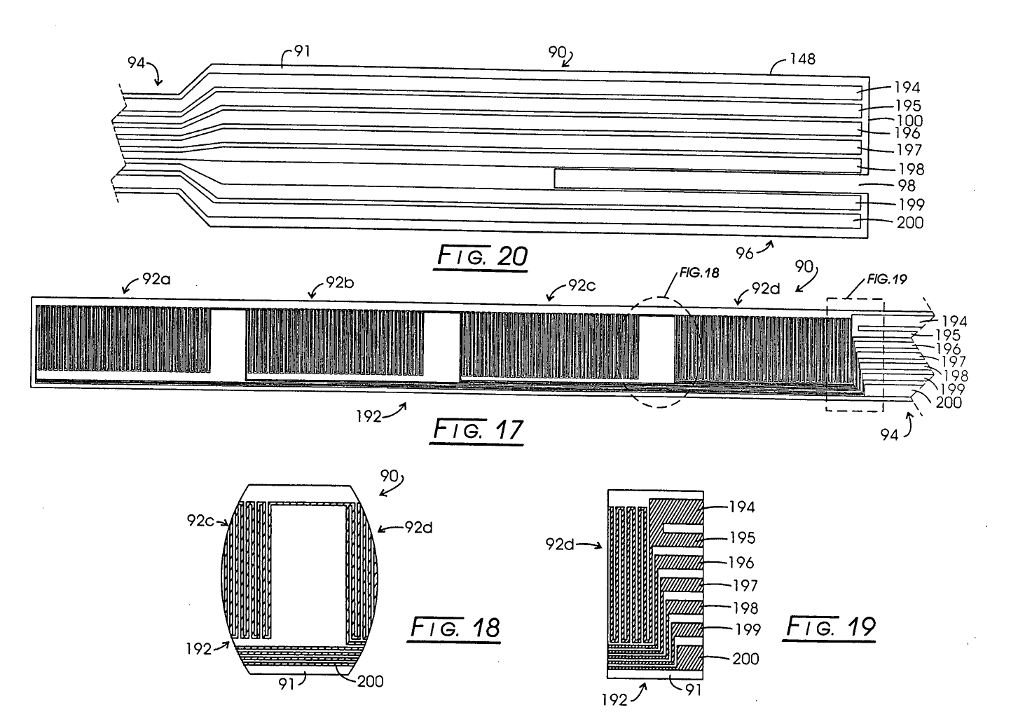

FIGS. 13 and 14 illustrate portions of flex circuit 110 and, in particular, the lead structure extending to active electrodes 34a-34d. In FIG. 13 an initial lead region is represented in general at 160 and is comprised of quite thin width leads 162-165 extending from respective connection with electrodes 34a-34d. Leads 162-165 within region 160 exhibit a width of about 0.010 inch and a lead spacing of about 0.005 inch. From region 110, the leads extend to region 166 where their width expands to about 0.040 inch. That widthwise extent continues into the contact region 116 as seen in FIG. 14.

Referring to FIGS. 15 and 16, an enlargement of the electrode-bearing flex circuit for wands carrying the parallel segmented electrodes as at 50 is revealed at an enlarged level of detail. In the figure, an initial lead region is represented generally at 170. Within that lead region 170, leads 172-175 extend respectively to parallel segmented electrode structures 54a-54d. These leads have the same dimension and spacing as described above in connection with region 110 in FIG. 13. Rearwardly of region 170, the electrode leads expand in width at a region 176. The widthwise extent of the leads in region 176 corresponds with those described in connection with region 166 illustrated in FIGS. 13 and 14. The leads then continue to a contact region represented generally at 178 which is configured for engagement with the noted cable connectors as at 64 or 44. Note the presence of a cable connector registration slot component 180.

FIGS. 17-20 illustrate portions of the temperature sensing resistor carrying flex circuit 90 at an enlarged level of detail. Flex circuit 90 is configured with a polyimide (Kapton) substrate 91 having a thickness of about 0.001 inch. Resistor segments 92a-92d are configured and arranged to reside directly beneath in coincidence with the electrode structures of the electrode flex circuit. Accordingly, they will have a length along the wand or implant axis of about 10 mm. Additionally, the serpentine resistor segments will have an overall width of 0.151 inch and the serpentine traces will exhibit a width of about 0.003 inch. Those dimensions hold true within the resistor segment region represented generally at 192. With the geometry disclosed in connection with FIG. 8, the resistor segments 92a-92d are in thermal transfer relationship with corresponding electrodes so as to be effective to evaluate the temperature of those electrodes. These four sensing resistor segments are addressed by lead traces 194-200 which are arranged to provide a 4-point interconnection. In this regard, lead traces 194 and 200 provide a low level d.c. source current while leads 195-199 serve to provide temperature sensor outputs. Note that the widths of leads 194-200 are expanded within region 94 as represented in connection with FIGS. 19 and 20. In FIG. 20 the expanded width leads are seen to extend into the contact region 96 which is configured for compatible electrical union with cable connectors as identified at 44 and 64 in connection with respective FIGS. 4 and 6.

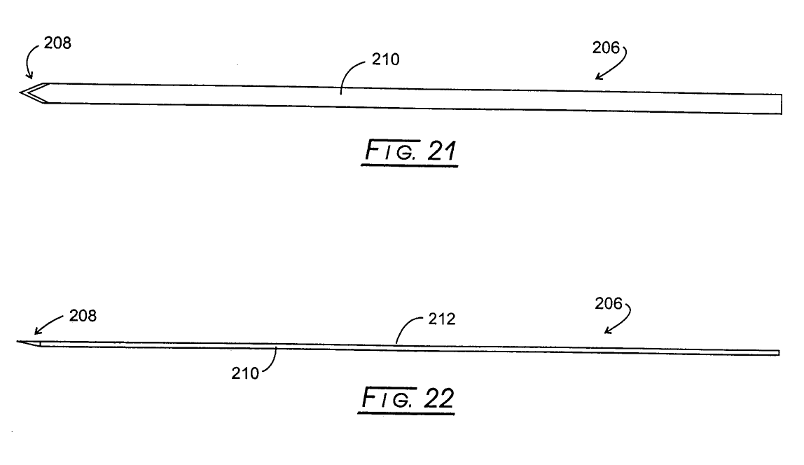

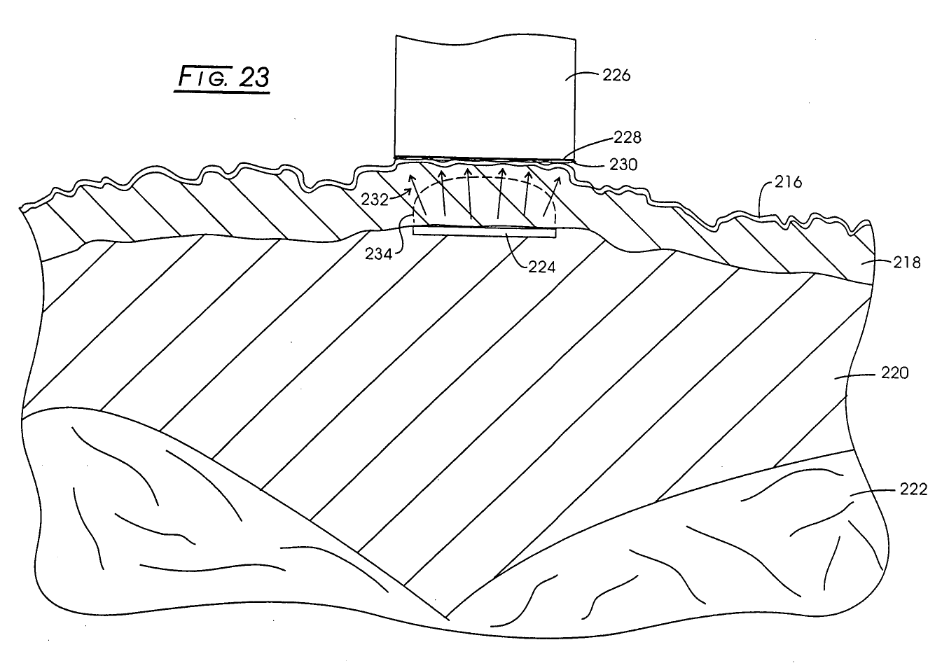

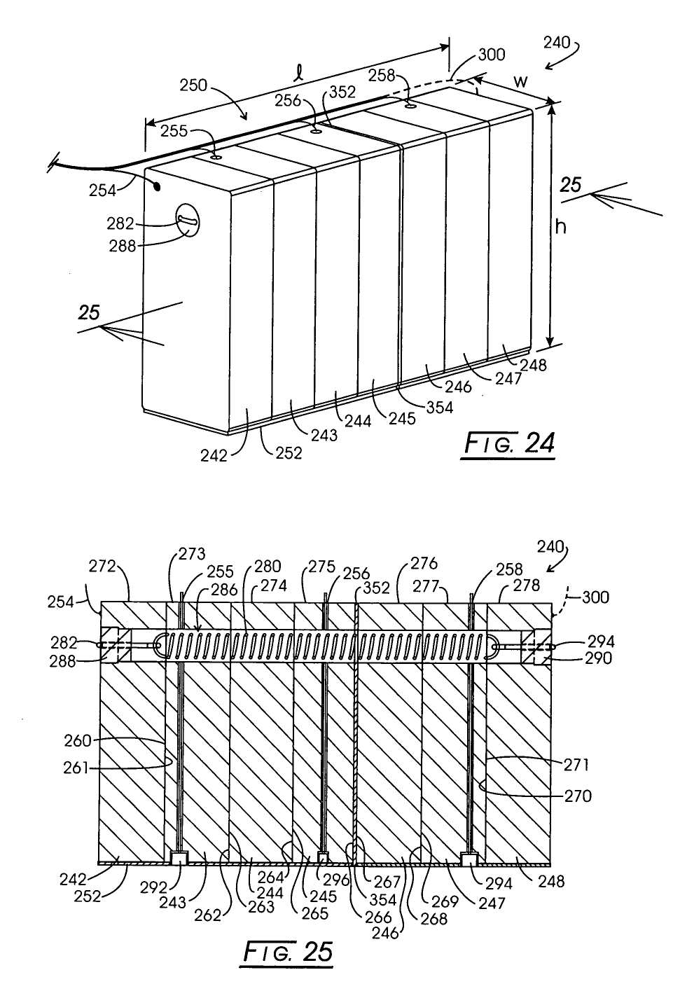

The positioning of implants or wands as at 30 and 50 at the interface between dermis and the next subcutaneous tissue layer involves a preliminary formation of a heating channel utilizing a flat needle introducer or blunt dissector. Looking to FIG. 21, such an introducer is represented generally at 206. Device 206 is, for instance, about 5 mm wide and is formed of a stainless steel, for example, type 304 having a thickness of about 0.020 inch to about 0.060 inch. Its tip, represented generally at 208 is not “surgically sharp” in consequence of the nature of the noted interface between dermis and fat layer. However, looking to FIG. 22, it may be observed that the tip 208 slants upwardly from the bottom surface 210 of device 206. This evokes a slight mechanical bias toward dermis when the instrument is utilized for the formation of a heating channel. In utilizing an introducer as at 206, the introducer is employed to form a heating channel from a scalpel formed entrance incision. Following placement and formation of a heating channel, a wand or implant is slid over the top surface 212 of the introducer. Upon positioning the implant or wand, then the introducer 206 is removed leaving the implant or wand in place. Alternatively, the heating channel can be formed following which the introducer 206 is removed and then a wand or implant is inserted in the thus formed heating channel.

Referring to FIG. 23, a sectional view of animal (pig) flesh is presented which schematically shows epidermis 216; dermis 218; next subcutaneous or fat layer 220; and underlying muscle 222. An introducer will have been employed to form a heating channel in which a wand or implant will have been inserted as represented schematically at 224. As noted above, that wand will carry four heating active electrodes as discussed in connection with FIGS. 4 and 6. Over these four electrodes there is positioned a return electrode and heat sink apparatus configured with aluminum blocks, an end block of which is represented at 226. The heat sink and return electrode apparatus will have a somewhat conformal contact surface 228 which is seen to extend slightly beyond the edges of the thermal wand 224. Of importance, there is located an electrically and thermally conductive material 230 between contact surface 228 and the surface of epidermis 216. Preferably, that material is isotonic saline solution. When the four electrodes of the thermal wand 224 are excited, R.F. current passes from them toward contact surface 228 as depicted by an arrow array represented generally at 232. This produces a treatment or heating zone represented by the dashed profile 234. Accordingly, the R.F. energy is utilized in a transdermal quasi bipolar fashion. It is of further importance that the contact surface 228 be accurately aligned with these four active electrodes. Wands as at 30 and 50 as described in connection with respective FIGS. 4 and 6 may be located at two or more positions within a heating channel such that excitation may take place at a furtherest distance within the channel from the entrance incision and then withdrawn to a second position within the same heating channel whereupon the active electrode excitation is repeated. Generally, it is desirable that assurance be made that the isotonic saline solution or equivalent be replenished for the second location elected for active electrode excitation. It may be noted that the material or isotonic saline solution 230 is both thermally and electrically conductive. Thus, there is no capacitive coupling involved with the present system. Were material 230 not employed, the resistance at the contact surface would be about 4,000 to 5,000 ohms at 350 kHz. With the location of isotonic saline solution between the epidermis surface and the contact surface of the heat sink and return electrode, the resistance reverts to a range of about 70 to about 180 ohms. Essentially all of the resistance involved resides at the dermis and the therapy interval can be quite short, for example around 65 seconds. Improved thermal conductance is achieved. Some purely monopolar systems employed capacitance coupling for an active electrode which is placed upon the surface of the epidermis. That is not desirable for the instant quasi bipolar system, inasmuch as capacitive coupling would require much higher voltage to achieve an adequate current level.

Looking to FIG. 24, a return electrode and heat sink apparatus is illustrated in perspective fashion and identified in general at 240. Device 240 is formed of seven generally rectangular thermally and electrically conductive material blocks 242-248. When combined together, the length, 1, of the sequence of blocks 242-248 will be about 3.75 inches; the width of the sequence, w, will be about 0.75 inch; and the height of each block, h, will be about 1.5 inches. End blocks are shown at 242 and 248 and for an embodiment not involving a patient circuit safety monitor feature, a terminal assembly is represented in general at 250. The contact surface of device 240 is provided as a thin flexible thermally and electrically conductive shim 252 fixed to and electrically interconnecting the sequence of blocks 242-248 while permitting their slideable movement to generally accommodate a given contour of the skin surface of the patient. The shim 252 may be formed, for example, of copper. For the instant embodiment, the R.F. electrical return component of terminal assembly 250 is shown at lead 254 electrically engaging end block 242. Terminal assembly 250 also includes a lead 255 extending within a slanted bore within end block 242 which receives an output from a photo-detector adjacent contact surface or shim 252. Another lead as at 256 extends within a slanted bore to a thermocouple located adjacent the shim-defined contact surface 252. In this regard, a thermocouple is utilized in view of its more rapid response time. Next, a lead 258 extends to a photo-detector through a slanted bore.

Looking to FIG. 25, a sectional view of device 240 is revealed showing that the blocks 242-248 are configured with flat sliding surfaces 260-271 which extend between an outer surface 272-278 and a receiving surface adjacent shim 252. Shim 252 is connected with this receiving surface with a thermally and electrically conductive adhesive. Blocks 242-248 are retained together adjacent outer surfaces 272-278 by a tension retainer such as a coil spring 280. Spring 280 is seen to extend in tension between hooks 282 and 284 within a sequence of mutually aligned bores represented generally at 286. Hooks 282 and 284 are mounted within respective end caps 288 and 290 located within end blocks 242 and 248. With the arrangement shown, the blocks 242-248 are arranged in a freely abutting relationship such that their slide surfaces may move slightly, albeit constrained by the shim 252 such that the device 240 may conform to the contours of the skin surface being treated. Spring 280 may be replaced with an electrically insulative elastameric band.

As noted above, it is important that the combined return electrode and heat sink 240 be disposed directly over and in alignment with the four electrodes, for example, as described at 34a-34d in FIG. 4 and 54a-54d in FIG. 6. Misalignment may result in thermal trauma to the skin resulting from a lack of the heat sink function. In general, device 240 is designed with blocks 242-248 formed of aluminum and, as a sequence, defining a thermal mass effective to avoid thermal trauma to epidermis. One approach to assuring proper alignment with the four electrodes of a wand as at 30 or 50 is to provide what may be termed an electronic interlock wherein photo-detectors are centered along the width, w, of device 240 and are spaced apart a distance matching the space between the two light emitting diodes, for example, as described in FIG. 4 at 38 and 40 and in FIG. 6 at 58 and 60. That spacing will be about 47 mm. In FIG. 25, leads 254 are seen extending to a photo-detector such as a photodiode 292. Similarly, leads 258 extending through a slanted bore in block 247 extend to communication with a photo-detector 294. Photo-detectors 292 and 294 are spaced apart about 47 mm. Leads 256 within block 245 extend to a thermocouple 296 mounted in thermal exchange relationship with shim 252. Control can be provided from a controller to develop an oral warning cue should no signal be provided from either photo-detector 292 or 294. Such a controller also may be programmed to shut down in addition to providing an oral cue.

The return electrode function of device 240 also can be configured such that, in combination with a controller, a patient circuit safety monitor (PCSM) function may be carried out. To do this, the electrical return function of device 240, is divided into two components which are mutually electrically isolated. This requires an additional return terminal for the assemblage 250 as shown in dashed line fashion at lead 300. Additionally, an electrically insulative divider is positioned between blocks 246 and 245 as shown in FIGS. 24 and 25 at 352. Leads 254 and 300 additionally function as monitors for the PCSM function and the shim 252 is split at location 354. During the test function, leads 254 and 300 are utilized to output a high frequency current, which is directed from one electrically insolated block sequence to the other. In this regard, the PCSM circuit will apply about a 10 volt signal at 50 KHz to the two return electrode regions and verify proper resistance. In general, only upon such verification will the control system permit the practitioner to continue the procedure. For a detailed description of PCSM functions, see U.S. Pat. No. 6,923,804 by Eggers, et al. In general, the sides of device 240 are covered or coated with an electrically insulative material permitting it to be hand-held by a practitioner.

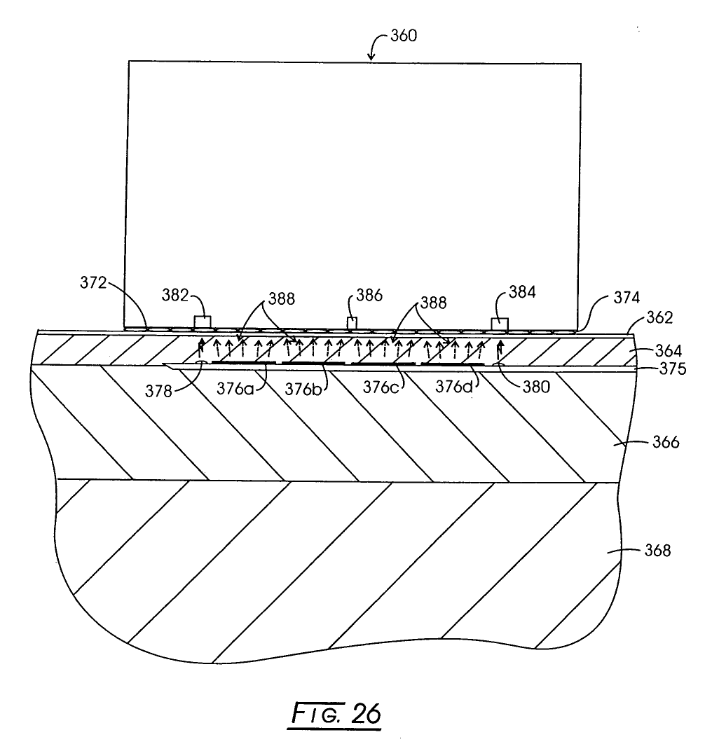

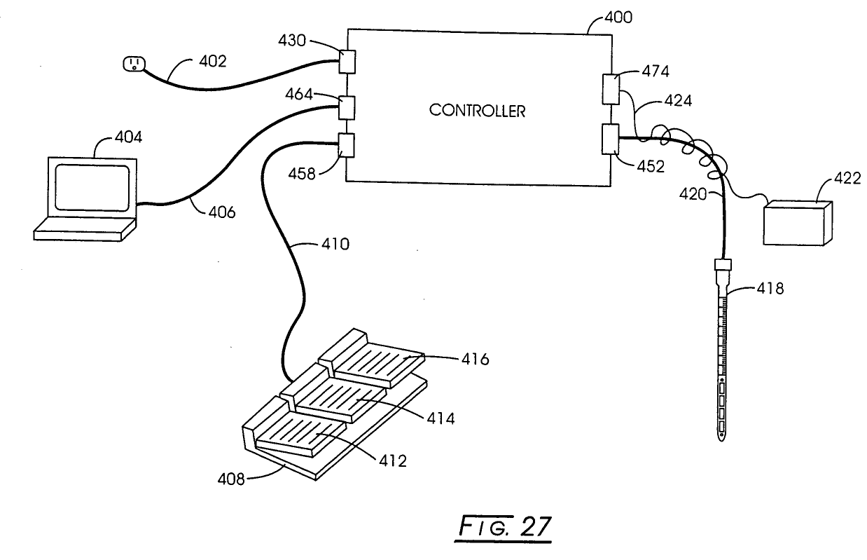

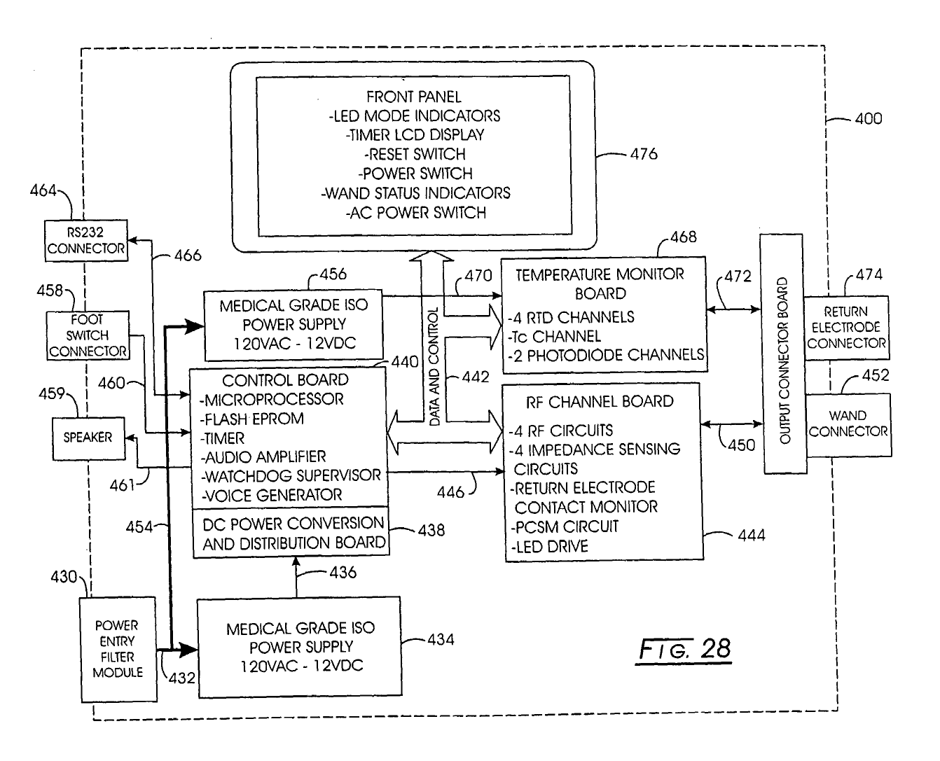

The return electrode and heat sink function may be carried out with a solid block formed of electrically and thermally conductive material such as aluminum. Referring to FIG. 26, such a solid combined return electrode and heat sink is represented in general at 360. Block 360 is rectangular with generally the same dimensions as device 240, preferably being formed of aluminum with a thermal mask effective to carry out the heat sink function. Block 360 is illustrated in conjunction with a schematically depicted tissue section including epidermis 362; dermis 364; next subcutaneous tissue layer or fat layer 366; and underlying muscle 368. A thermally and electrically conductive material such as isotonic saline solution is symbolically represented at 370 located between the contact surface 372 and the surface of epidermis 362. A thermal wand or implant is shown in general at 374 located at the interface between dermis 364 and fat layer 366. Four R.F. energized active electrodes 376a-376d are shown to be carried by the implant 375 in addition to two red region light emitting diodes 378 and 380. LED 378 is shown to be energized and emitting toward an aligned and centrally disposed photo-detector 382. In similar fashion, LED 380 is shown to be energized and aligned with centrally disposed photo-detector 384. Accordingly, at the loss of a signal from either of the photo-detectors 382 or 384, a verbal cue may be given to the practitioner and, additionally, the procedure may be shut down to avoid thermal damage. Also disposed centrally within device 360 and in adjacency with contact surface 372 is a thermocouple 386. R.F. current flow from active electrodes 376a-376d is represented by arrow arrays shown in general at 388.

Temperature evaluating resistor segments have been discussed, inter alia, in conjunction with FIGS. 8 and 17-20. Considering the functioning of these segments, once a wand or implant has been located within a heating channel and preferably with application of the heat sink function, the temperature of resistor segments is determined. For example, this predetermined resistor segment temperature, T_{RS, t0}, based on an algorithm related to the measured skin surface temperature, T_{skin, t0}, may be expressed as follows:

T_{RS,t0}=f(T_{skin,t0}). (2)

As an example, this computed temperature may be 35° C. Also predetermined are the treatment threshold or setpoint temperature as well as high or limit temperature, and optionally, lower limit temperature, and optionally lower limit temperature.

When the controller is instructed to commence auto-calibration the following procedure may be carried out:
a. The controller measures the resistance of each resistor segment preferably employing a low-current DC resistance measurement to prevent current induced heating of those resistors.
b. Since the resistor component is metal having a well-known, consistent and large temperature coefficient of resistance, α having a value preferably greater than 3000 ppm/° C. (a preferred value is 3800 ppm/° C.), then the target resistance for each Resistor Segment can be calculated using the relationship:

R_{RSi,target}=R_{RSi,t0}(1+α*(T_{RS,t}−Tₜₒ)) (3)
where:
R_{RSi,t0}=measured resistance of Resistor Segment, i, at imputed temperature of Resistor Segment under skin, T_{RS,to}
α=temperature coefficient of resistance of resistor segment.
T_{RS,t}=target or setpoint treatment temperature.
T_{RS,t0}=Imputed temperature of RF electrodes residing under the skin and prior to the start of any heating of them.

Concerning the imputed temperature of a resistor segment under the skin, their actual temperature is estimated based upon the read-out of a temperature sensor such as a thermocouple on the skin surface with an added estimated temperature increment. That added increment evokes an estimated temperature value at the dermis-subcutaneous fat layer (39) interface. Alternately, a temperature sensor such as a sheathed thermocouple can be temporarily inserted along the heating channel over a wand over an electrode and underlying resistor to obtain a resistance/temperature relationship.

For four-point sensor resistor connections, no accommodation need be made for the impedance exhibited by the cable extending to the controller. Temperature evaluations are made intermittently. For instance, during a continuous or ramp-up mode of performance they may be made every 500 milliseconds and a sampling interval may be quite short, for instance, two milliseconds. For intermittent mode performance, the interval for temperature management in voltage control may be approximately one second with respect to the measurement of temperature of all electrodes involved. Again, the sampling interval may be quite short, for example, two milliseconds.

Referring to FIG. 27, a pictorial representation of the system at hand is presented. In the figure, a controller is represented symbolically at 400. This controller is provided conventional a.c. power through a hospital grade power cord represented at 402. As represented at laptop computer symbol 404 and serial cable 406, the controller 400 is seen to be associated with a computer display and interactive association with an external computer. Wand electrode energization under timing constraint is activated by the practitioner, for example, utilizing a three-pedal foot switch symbolically represented at 408 as is associated with controller inputting cable 410. The foot activated switches at symbol 408 include a reset switch 412, a heat enable switch 414, and a heat application switch 416. The thermal wand or implant is represented at symbol 418 as being associated with controller 400 from cable 420. Finally, the return electrode for this quasi-bipolar transdermal dermis region heating approach is represented symbolically at 422 with the return electrode cabling being represented at 424 extending to controller 400.

Now looking to the controller itself, reference is made to FIG. 28 where the controller function is represented within dashed boundary 400. Utility power is supplied to the controller 400 at a power entry filter module represented at block 430. As represented at arrow 432 and block 434 the filtered input is treated by a medical grade power factor correction form of network power supply deriving a 28 volt d.c. output as represented at arrow 436. Such a power factor correction derives a universality to controller 400, for instance, permitting its use in off-shore locations with different electrical utility parameters. Arrow 436 is seen to be directed to a d.c. power conversion and distribution board represented at block 438. Board 438 supplies appropriate levels of d.c. input to a control board represented at block 440 which carries, for example, a microprocessor function; a flash EPROM function; a timer function; an audio amplifier function employed with a voice enerator providing vocal cues to the operator; and a conventional watchdog supervisor function. Those functions are associated with a data and control busing organization represented at bus symbol 442. That bus function 442 is seen associated with a radiofrequency channel board represented at block 444 as is the control board 440 as represented at arrow 446. The radiofrequency channel board 444 incorporates four radiofrequency energization circuits and four impedance sensing circuits which monitor the above-discussed resistor segments utilized for monitoring the temperature of the active electrodes. The function of block 444 additionally includes a return electrode contact monitor function. It may be observed that current flow from the active electrodes will shut down if the return electrode is removed from the surface of the epidermis. Additionally, this board will carry a PCSM circuit as above-described and, additionally a drive circuit for the light emitting diode function associated with each wand or implant. Board 444 is shown interactively associated with an output connector board represented at block 448 as represented at interactive or dual arrow 450. Connector board 448 is seen to be electrically operationally associated with a wand or implant connector as represented at block 452.

Returning to arrow 432, a diversionary arrow 454 is seen extending to block 456 representing a medical grade ISO power supply which develops a 12 v d.c. output. Note that control board 440 is activated from footswitch 408 as represented by a footswitch connector at block 458 as associated with arrow 460. The function at block 440 additionally is interactively associated with an external computer function as described at 404 as represented at R.S. 232 connector function block 464 and dual or interactive arrow 466. Also, control board 440 drives a speaker as represented at block 459 and arrow 461. In this regard, the microprocessor or microcontroller function may direct A law compressed voice data to a general purpose signal channel PCM CODEC. The latter device may be provided as a type W681310 device marketed by Winbond Electronics Corp., of Hfinchu, Taiwan. This device carries out a decompression function or decoding to evolve analog human voice signals providing a voice message output which may be volume controlled. In this regard, it may be observed that the operator of the system typically will be hand-holding a heat sink as described in connection with FIGS. 24 and 26 in position over the electrodes of the implant or wand.

The power supply as represented at block 456 distributes d.c. input to a temperature monitor board represented at block 468 as indicated by arrow 470. Such function as represented at block 468 provides for four RTD channels which monitor temperature responsive resistor segments; thermocouple channel; and two photodiode or photo-detector channels. The function as represented at block 468 is interactively associated with the output connector board 448 as represented by dual arrow 472. Note additionally, that the output function at block 448 is associated with the return electrode function as represented at block 474.

The data and control busing function 442 also is seen associated with the temperature monitoring board 468 and, additionally, extends to a controller front panel function represented symbolically at 476. That front panel will incorporate light emitting diode mode indicators; timer liquid crystal displays; a reset switch; a power switch; wand or implant status indicators; and an AC power switch.

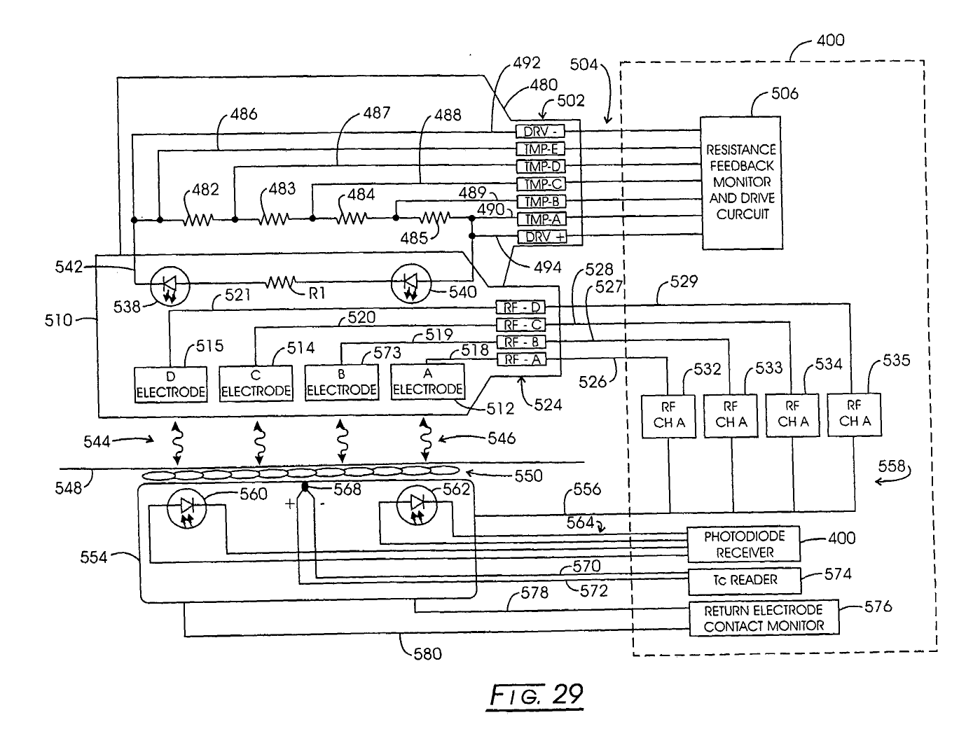

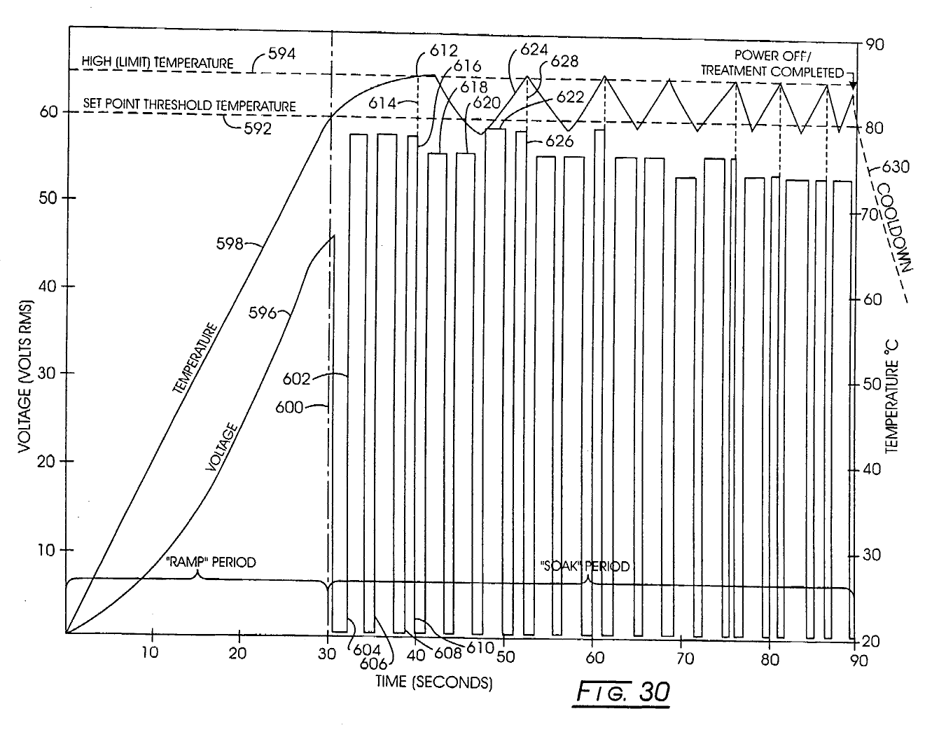

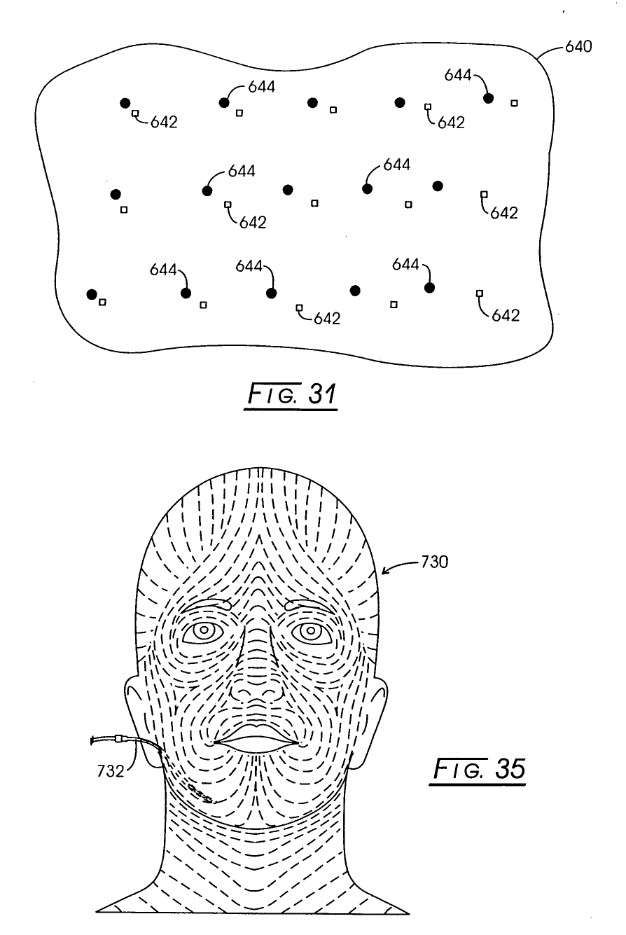

Referring to FIG. 29, a schematic representation is provided showing the association of the temperature sensing resistor flex circuit function as discussed in connection with FIGS. 17-20 with the controller function 400; the association of the active electrode carrying flex circuit as described in connection with FIGS. 13-16 with the controller function 400; and the association with the combined return electrode and heat sink function with controller function 400. As noted earlier, the resistor segment temperature sensors are associated with the controller function through a four-point coupling which functions to sample temperature intermittently both during active electrode energization and during cool-down periods. The four resistor segment temperature monitors are represented at 482-485 as associated with leads 486-490. Four-point connector drives are shown at leads 492 and 494. These leads extend to a cable connector contact array represented generally at 502. From these contacts, as represented at lead array 504, connection is made with a resistance feedback monitor and drive circuit shown at block 506 within controller 400.

Associated with the temperature monitoring flex circuit 480 is the active electrode carrying flexible circuit now represented symbolically at 510. Within that symbol the active radiofrequency energized electrodes are identified at 512-514 and further represented by respective alphabetical designations A-D. Lead inputs to these electrodes are represented at respective lines 518-521 which extend to cable connector contacts having corresponding alphabetical designations and represented at a contact array identified in general at 524. From the contact array 524, cable leads 526-529 extend to the controller 400 and corresponding radiofrequency excitation channels represented at respective blocks 532-535. Note additionally, that the active electrode carrying flexible circuit 510 also functions to energize the alignment light emitting diodes. In this regard, inasmuch as these devices are of relatively low voltage, they can be coupled in series circuit fashion. These LEDs are represented symbolically at 538 and 540, connected within a lead 542 incorporating resistor R1 and coupled with flex circuit source or drive leads 492 and 494. Dermis is represented in general at 544 and R.F. energy is represented at a symbol array shown generally at 546. Epidermis is represented at line 548 and a layer of isotonic saline solution is represented symbolically in general at 550. The combined return electrode and heat sink is symbolically represented at 554. This device is shown incorporating a return electrode function with a cable lead 556 extending to a lead array represented in general at 558 providing a common return connection to radiofrequency channels 532-535. A photo-detector function as represented by photodiode symbols 560 and 562 is provided within the heat sink function 554. Note that they are aligned with LEDs 538 and 540. Photodiodes 560 and 562 are coupled by a lead array represented generally at 564 to a photodiode receiver function represented at block 566 within controller 400. Any loss of signal from devices 560 and 562 will represent a misalignment of the return electrode and heat sink function to generate voice cueing. The earlier-described thermocouple is symbolically represented at 568 in conjunction with dual leads 570 and 572 extending to a thermocouple reader function represented at block 574 within controller 400. Also within that controller designation is a return electrode contact monitor as well as a PCSM circuit as represented at block 576 and leads 578 and 580. Typically, the PCSM circuit will be coupled to the return electrode function via terminal assemblies associated with cable lead 556.

Controller 400 performs in an manner at least in part wherein active electrode excitation takes place during power-on intervals spaced apart in time by power-off intervals. These intervals are associated with a setpoint temperature threshold as well as an upper limit high temperature level. This approach has the advantage of achieving dermis treatment for treating skin laxity and the like over quite a short interval. Following this pulse-like treatment interval which may amount to 60 or 65 seconds, a cool-down interval ensues wherein the heat sink function is maintained in contact with the epidermis surface to absorb residual dermis heating and protect the epidermis. Referring to FIG. 30, procedure time in seconds is plotted along an abscissa; applied voltage (volts RMS) is plotted along a leftward ordinate; and temperature in degrees centigrade is plotted along a rightward ordinate. A setpoint threshold temperature may be set by the operator, for example, at 80° C. as represented by horizontal dashed line 592. Additionally set is a high or limit temperature as represented at horizontal line 594 representing an 85° C. level. Voltage associated with the radiofrequency energization process is plotted at curve 596, and the temperature of the electrodes as monitored by the above-noted resistor segments is plotted at curve 598. At the commencement of the procedure, controller 400 modulates voltage in a ramping fashion generally occurring within the first thirty seconds from startup. This is referred to as a “ramp” which terminates as represented at vertical dashed line 600. Note that it is at this same time or ramp period, the temperature has risen to the setpoint threshold temperature of 80° C. RMS voltage then drops to zero whereupon following a pre-designated power-off time interval a nominal maximum voltage pulse having a width as preset by the controller 400 software is carried out to define a power-on time, for example, of two second duration. This power-on time is represented by the voltage pulse 602. Note that during this interval, curve 598 is passing the setpoint threshold temperature level 592. As represented at pulse 604, a timed power-off interval ensues having a duration, for example, of one second. During this power-off time, the temperature responsive resistor segments may be polled to determine electrode temperature. The programmed power-on time then is repeated as represented at pulse line 606, again being programmed for full voltage assertion for a programmed power-on interval, for example, two seconds. Note that the temperature curve 598 continues to rise toward high temperature level 594. At the termination of this next power-on interval, as represented at pulse line 608, a programmed power-off interval ensues, for example, of one second. Note that temperature curve 598 continues to ascend toward the high temperature level at dashed line 594 following this next power-off interval. The next power-on pulse commences as represented at pulse line 610. However, during a programmed power-on interval represented by that pulse, the temperature level will have reached the high limit temperature represented at dashed line 594 as represented at position 612. As illustrated by dotted line 614 and pulse line 616, the power-on interval is terminated and as represented at pulse levels 618 and 620, voltage amplitude is adjusted downwardly to permit the electrode temperature to drop as is shown slightly below the setpoint threshold temperature represented at dashed line 592. A power-off interval ensues whereupon as represented by pulse 622, a next power-on interval is one under full voltage conditions causing an elevation in temperature as represented at curve component 624 of temperature curve 598. Following a next power-off interval, a full power pulse voltage occurs as represented at 626, however, as represented at dotted line 628, the power-on interval of pulse 626 is terminated early. This procedure continues until the completion of therapy, which is shown to occur at 90 seconds, at which point the voltage is terminated. However, the operator is cued to maintain the heat sink function in place for a cool-down interval of, for example, thirty seconds as represented at dashed line 630. The interval shown in the figure extending from thirty seconds to ninety seconds generally is referred to as a “soak period”.

The energization mode represented in FIG. 30 was utilized in carrying out animal (pig) studies which resulted in a desirable dermis shrinkage occurring over the noted relatively short therapy intervals. Looking to FIG. 31, a sketch reproduction of a digital image of an in vivo animal test is represented within boundary 640. The epidermis surface initially was marked with a starting pattern of dots, herein represented as small squares, certain of which are identified at 642. Following a 65 second soak interval of treatment, the dot pattern shifted to represent shrinkage as illustrated at black circles, certain of which are represented at 644. Such shrinkage represents an average contraction along the length of the thermal wands utilized of 11%. In general, the animal experiments were carried out in connection with a surgical manipulation of the skin region tested to simulate skin laxity or the like. Histopathology carried out with the tissue samples collected showed in some cases, either an epidermal burn or thermal damage to the muscle layer, such layers being depicted at 222 in connection with FIGS. 23 and 368 in connection with FIG. 26.

The burn injury observed in histopathology analysis may have been caused by at least one of the following:

1. Conduction of heat i.e., from the actively heated side of the thermal wand (which faces the dermis) to the underlying subcutaneous fat layer and muscle layer.

2. Conduction of R.F. electrical current from the R.F. electrode, through the subcutaneous fat layer to the muscle layer. In this possible cause, errant R.F. electrical current spreads into the muscle layer and then flows back through the subcutaneous fat layer, dermis and epidermis over a much larger area that corresponds to the total contact area of the return electrode/heat sink. As the R.F. electrical current flows through the muscle layer it can induce Joulean heating by virtue of the electrical resistance of the tissue.

With the above observation it was determined to provide an R.F. excitation of the active electrodes utilizing a multi-stage pulsed-mode heating method.

The purpose of this multi-stage pulse-mode heating method is based on two inter-related thermal doses. A first thermal dose is based on the thermal dose achieved within the dermis, which needs to be sufficiently high to effect aesthetically beneficial levels of skin contraction (preferably about 10% along the longitudinal axis of the thermal wand and observable at the time of treatment). This thermal dose is referred to as the “contraction thermal dose”.

The amount of time, t, that the collagen fibrils need to be exposed to elevated temperature, T, to achieve a desired collagen contraction level, S, has been previously reported by Wall (see publication 20) and is given by the empirically derived 7-parameter logistic equation (sigmoidal function) earlier described at equation (1).

The results of this experimental study reported by Wall demonstrated the exponential dependence of exposure time, t, on treatment temperature, T. Hence, the required level of contraction can be achieved in a relatively short period of time if the collagen fibrils are raised to a sufficiently high temperature of about 70° C. or more. In addition, a sufficient thickness of collagen fibrils needs to be raised to an elevated temperature for the required duration in order to produce a contraction force sufficient to overcome the restorative forces in the surrounding untreated skin (i.e., a force sufficient to effect observable skin contraction). As reported by Rasmussen (publication no. 6), the level of contraction “tension” produced in freshly harvested human dermis samples (with epidermis and subcutaneous fat layers removed) raised to over 75° C. is directly proportional to the thickness of the dermis. Accordingly, if only a small fraction of the dermis exposed to a thermal dose sufficient to effect contraction (e.g., 10% of the total dermis thickness), the tension it will produce would only be one-fourth of the tension produced if 40% of the total dermis thickness is exposed to the same thermal dose.

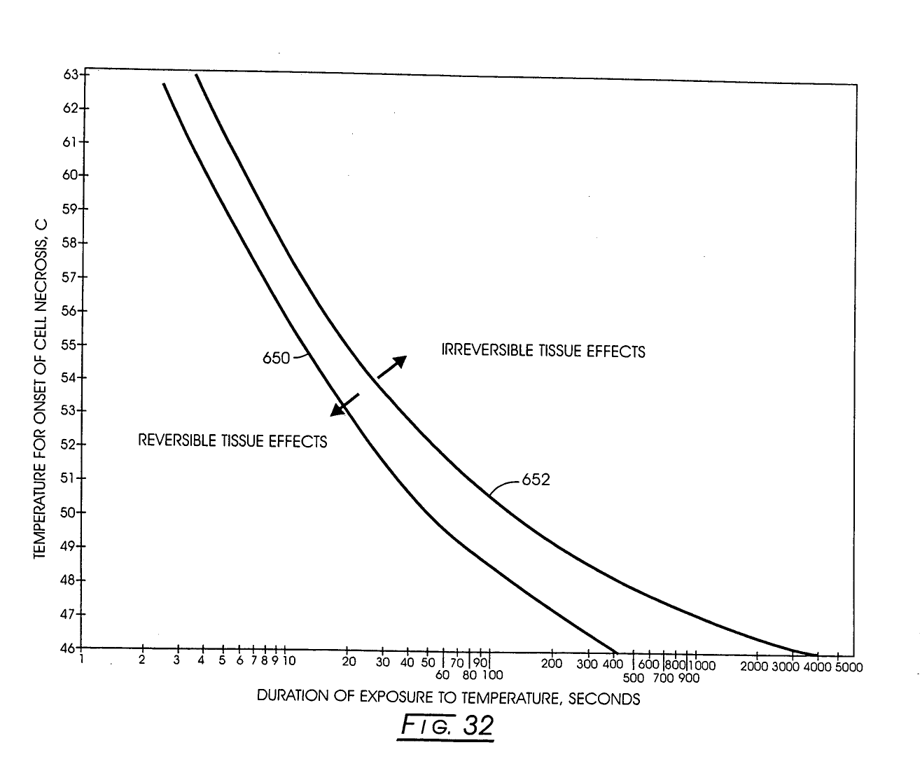

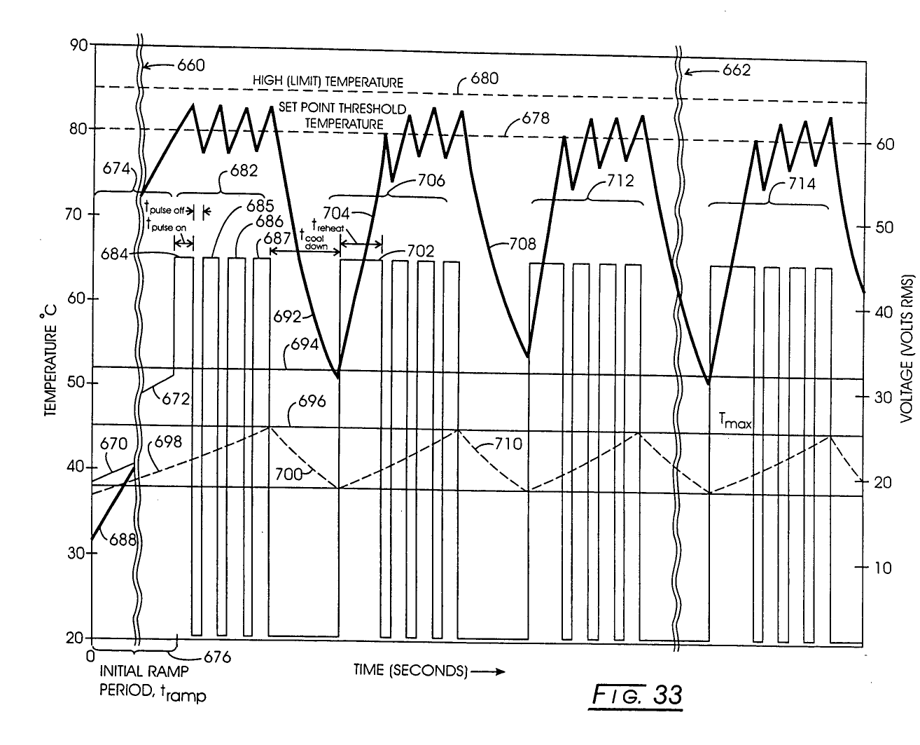

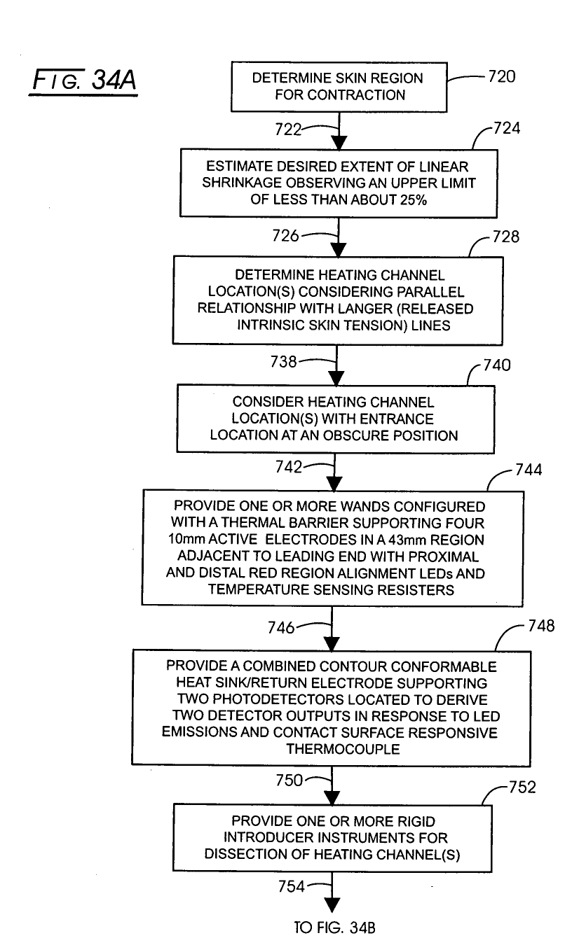

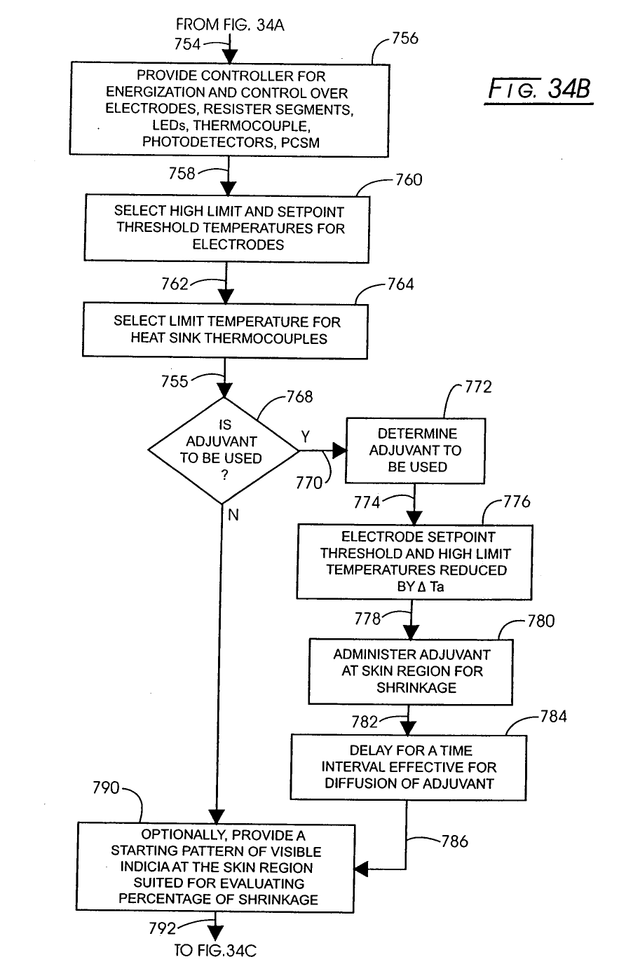

A second thermal dose of interest is the maximum thermal dose that the subcutaneous fat layer and muscle layer can withstand during the intradermal heating procedure without inducing irreversible cell death (also referred to as “acute coagulative necrosis”). This thermal dose has been referred to as the “safe limit thermal dose”. The maximum allowed exposure time of tissue (including the more thermally sensitive nerve tissue) to elevated temperatures is presented in FIG. 32. For example, as seen in that figure, the subcutaneous fat layer, muscle layer and nerve tissue can be protected from irreversible injury for total exposure periods of 60 seconds if the maximum exposure temperature is maintained below about 50° C. In the figure, the region to the left of nerve tissue curve 650 and other tissue curve 652 represents reversible tissue effects, while the curve region to the right of these curves represents irreversible tissue effects. The sources for evolving these curves are as follows:
25. Henriques, F., Studies of Thermal Injury, Arch. Path., 1947, 43, pp 489-502.
26. Niemz M., Laser-Tissue Interactions, Springer-Verlag, Berlin, 2002, pg 78.
27. Eichler, H. and Seller, T., Lasertechnik in der Medizin, Springer-Verlag, Berlin 1991.

In view of the forgoing it is necessary to achieve both (1) the contraction thermal dose necessary to affect aesthetically acceptable levels of skin contraction and (2) a safe limit thermal dose which avoids clinically significant thermal injury to the subcutaneous fat layer and muscle layer underlying the dermis.

The intermittent or pulse node of energization described in connection with FIG. 30 was originally developed to avoid burn injury to the epidermis. In this heating mode, power is applied for a brief period (viz., the power-on) of 1 to 4 seconds followed by a period of no power application (viz., the power-off interval) of 1 to 2 seconds. This pulsed or intermittent form of intradermal heating has been shown to be effective in achieving target skin contraction levels of at least 10% (parallel to the longitudinal axis of the implant or wand) while avoiding thermal injury to the epidermis. This is represented at FIG. 31. However, this intermittent or pulse mode of heating does not necessarily prevent irreversible burn injury to the muscle layer when the thickness of the subcutaneous fat layer underlying the implant or wand is about 2.5 mm or less.

An approach to avoiding clinically unacceptable irreversible burn injury to the muscle layer is to (1) lower the treatment temperature range (i.e., the range of temperatures reached during each power-on and power-off intervals of power application) and/or (2) or reduce the total treatment period. Either one or a combination of these approaches will unavoidably reduce the contraction thermal dose. Using one or both of these approaches to affect a sufficient reduction in the safe limit thermal dose to prevent clinically significant irreversible injury to the muscle layer (as well as the subcutaneous fat layer) will most likely reduce the contraction thermal dose to the point of unacceptably small skin contraction levels. This observation is based upon the fact that the contraction thermal dose identified in the course of acute in vivo pig experiments is close to the minimum contraction thermal dose required to achieve the target 10% contraction level. Consequently, any reduction in this contraction thermal dose can only serve to reduce the skin contraction level below 10%.

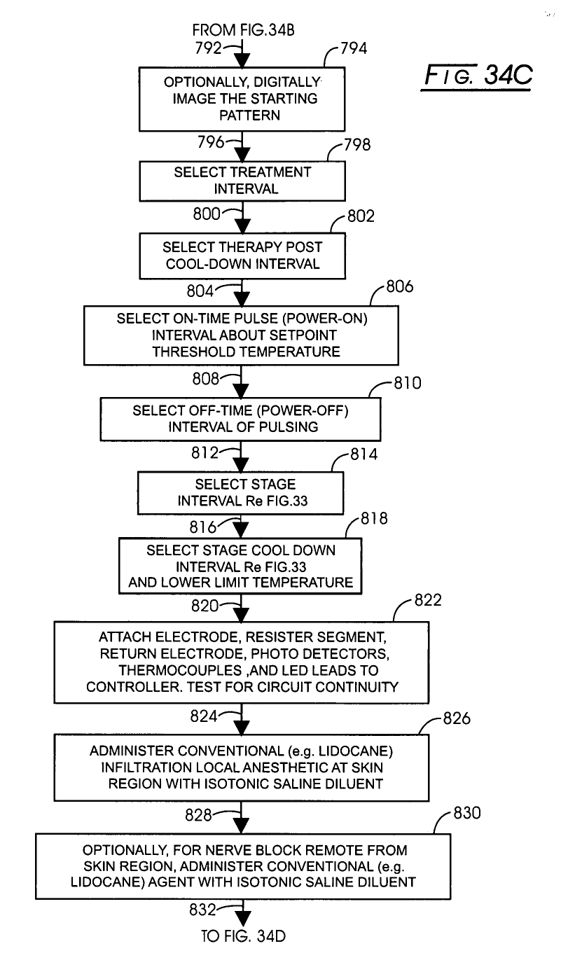

An alternative approach to achieving clinically acceptable levels of both the contraction thermal dose and the safe limit thermal dose is illustrated in connection with FIG. 33. In the convenience of scale, the figure is presented with two broken away regions represented generally at 660 and 662. In the figure, time in seconds is represented along the abscissa. Temperature in degrees centigrade with respect to the active electrodes is represented at the left ordinate and applied voltage (volts RMS) is represented in the right abscissa. The energization algorithm illustrated involves multiple stages of pulsed-mode treatments separated by port stage cool down period, t_{cool down}. The rationale for the critical cool down period, t_{cool down} is that it be just long enough to allow the temperature of the subcutaneous fat layer and muscle layer to cool down to a sufficiently low temperature that the next pulsed or intermittent mode treatment cycle will maintain these layers below the threshold thermal dose for irreversible burn injury. During the cooling period, t_{cool down} the vascular nature of the muscle layer serves as a heat sink to cool down these muscle tissue as a result of the flow of perfused blood which is at a temperature of about 37° C. As in the case of the ramp represented in FIG. 30, there is an initial voltage modulated ramp-up period represented by the voltage curve components 670 and 672. This is referred to as the ramp-up stage as represented at bracket 674 which occurs during the initial ramp period, tᵣₐₘₚ, as indicated at bracket 676. As in the case of FIG. 30, the practitioner will have set a setpoint threshold temperature, for example, of 80° C. as represented at horizontal dashed line 678 and a high limit temperature as represented at dashed line 680. The instant approach is similar to that described in connection with FIG. 30, however, the total treatment period during an initial stage 1 as represented at brackets 682 is much shorter, (e.g., 6-12 seconds). Note that the pulse form in stage 1 is at a full voltage level as represented at pulse voltage amplitude levels 684-687. Now looking to the electrode temperature values, elevation in temperature is represented by temperature curve components 688 and 690. Note as curve component 690 reaches the setpoint threshold temperature at dashed line 678, stage 1 as shown at bracket 682 commences. For each power-off interval, t pulse off, the temperature is seen to drop below setpoint threshold temperature as represented at line 678. Following the pulse shown at pulse amplitude 687, a cool-down interval ensues, t_{cool down}, as the temperature of the electrode drops significantly as represented at temperature curve component 692. This interval may be on the order of 6-15 seconds. The duration of the cooling period, t_{cool down}, may be a fixed time period or it may be based upon a predetermined lower limit level of the electrode temperature, T cool down, as represented at horizontal line 694. Below horizontal line 694 is a horizontal line 696 identified as, Tₘₐₓ, representing the maximum temperature to be permitted at the fat layer and muscle layer. In this regard, the temperature at those layers is plotted at dashed curve 698 which is seen to commence during the ramp-up period from a normal temperature of 37 C and will have reached the, Tₘₐₓ, level at line 696 at the termination of stage 1 as represented at bracket 682. During the, t_{cool down}, interval, the fat and muscle layers will be seen to return toward normal body temperature of 37° C. as represented by curve component 700. At the commencement of stage 2, a wider voltage pulse width as represented at pulse 702 is evolved which is referred to as, t reheat. Note that this pulse 702 terminates as electrode temperature reaches the setpoint threshold temperature at dashed line 678 as represented at curve component 704. When that temperature level is reached, the pulse 722 is terminated. This pulse interval may be accomplished as a shorter interval than a ramp-up period, i.e., of about 8 to 20 seconds since the electrode temperature at the start of this reheat period is well above the initial temperature of 30 to 35° C. Once this reheating as represented at pulse 702 is completed, the pulse or intermittent heating cycle is reinitiated. Stage 2 is represented at bracket 706 and during that stage the active electrode temperature is seen to move above and below the setpoint threshold temperature represented at dashed line 678. Following stage 2, a power-off interval, t_{cool down}, then ensues as active electrode temperature drops as shown at curve component 708. Note that during this interval temperature at the fat and muscle layer reduces as represented at dashed curve component 710. Following the power-off cool-down period, a stage 3 then ensues as represented at bracket 712. This procedure continues until stage N as represented at bracket 714. Following the total treatment interval, a power-off condition ensues as the practitioner maintains the heat sink upon the skin surface over the four active electrodes for an interval also referred to as post therapy cool-down which may continue for about thirty seconds or so. In general, the total number of stages will be based upon the attainment of the target skin contraction level. A summary of possible parameters for this multi-stage pulse-mode method for intradermal heating is provided in Table 1 below. The higher the target skin contraction level, the greater the number of treatment cycles or stages required. Note no. 1 in Table 1 provides that this temperature is not controlled since a fixed cooling period is used in this example. Note no. 2 provides that this temperature is not measured but is inferred from the results of in vivo animal experiments and subsequent histopathology analysis. Note no. 3 provides that this cooling period is variable and is based on a predetermined lower limit electrode temperature to be reached during the cooling period.

TABLE 1
RANGE OF PARAMETERS FOR MULTI-STAGE PULSED-MODE INTRADERMAL HEATING METHOD

| Parameter | Variable Name | Minimum Value | Maximum Value | Example 1 | Example 2 |
|---|---|---|---|---|---|
| Initial ramp up period, seconds | tᵣₐₘₚ | 15 | 45 | 30 | 30 |
| Duration of Pulse “On” period, seconds | t_{pulse on} | 1.0 | 3.0 | 2.0 | 2.0 |
| Duration of Pulse “Off” period, seconds | t_{pulse off} | 1.0 | 3.0 | 1.0 | 1.0 |
| Cooling period between stages, seconds | t_{cool down} | 6 | 15 | 8 | Variable (Note 3) |
| Duration of reheating period, seconds | tᵣₑₕₑₐₜ | 6 | 20 | 10 | 10 |
| Number of Pulses in each Stage | Nₚᵤₗₛₑₛ | 3 | 8 | 4 | 4 |
| Number of Stages | N_{stages} | 2 | 6 | 3 | 3 |
| Set Point temperature, C. | Tₛₚ | 70 | 80 | 80 | 80 |
| High Temperature, C. | T_{H} | 75 | 85 | 85 | 85 |
| Lower-limit electrode temperature during | T_{cool down} | 45 | 60 | ~53 (Note 1) | 52 |
| Cooling down period, C. | | | | | |
| Maximum temperature of muscle during | Tₘᵢₙ | 45 | 50 | ~45 (Note 2) | ~45 (Note 2) |
| Pulse-Mode heating cycle, C. | | | | | |

A number of substances have been identified that interact with the ECM of the dermis to alter the thermally responsive properties of the collagen fibers. As described herein, substances with such properties are termed “adjuvants”. It will be recognized by those skilled in the art of protein structural chemistry that the reduction in length of collagen fibers, i.e., shrinkage, is the result in part of an alteration of the physical structure of the molecular structure of the collagen fibers. The internal ultrastructure of collagen fibers, being comprised of tropocollagen molecules aggregated into collagen fibrils, and then aggregated further into even larger collagen fibers, is a result of complex interactions between the individual tropocollagen molecules, and between molecules associated with the collagen fibers, for example, elastin, and hyaluronan. The molecular forces of these interactions include covalent, ionic, disulphide, and hydrogen bonds; salt bridges; hydrophobic, van der Waals forces. In the context of the present disclosure, adjuvants are substances that are capable of inducing or assisting in the alteration of the physical arrangement of the molecules of the skin in order to induce, for instance shrinkage. With respect to collagen fibers, adjuvants are useful for altering the molecular forces including those hydrophilic and hydrophobic forces holding collagen and associated molecules in position, changing the conditions under which shrinkage of collagen can occur.

Protein molecules, such as collagen are maintained in a three dimensional arrangement by the above described molecular forces. The temperature of a molecule has a substantial effect on many of those molecular forces, particularly on relatively weaker forces such as hydrogen bonds. An increase in temperature may lead to thermal destabilization, i.e., melting, of the three dimensional structure of a protein. The temperature at which a structure melts is known as the thermal transformation temperature. In fact, irreversible denaturation of a protein, e.g., cooking, is a result of melting or otherwise disrupting the molecular forces maintaining the three dimensional structure of a protein to such an extent that that once heat is removed, the protein can no longer return to its initial three dimensional orientation. Collagen is stabilized in part by electrostatic interactions between and within collagen molecules, and in part by the stabilizing effect of other molecules serving to cement the molecules of the collagen fibers together. Stabilizing molecules may include proteins, polysaccharides (e.g., hyaluronan, chondroitin sulphate), and ions.

A persistent problem with existing methods of inducing collagen shrinkage that rely on heat is that there is a substantial risk of damaging and or killing adipose (fat layer) tissue underlying the dermis, resulting in deformation of the contours of the overlying tissues, with a substantial negative aesthetic effect. Underlying muscle tissue also may be adversely affected. Higher temperatures or larger quantities of energy applied to the living cells of the dermis can moreover result in irreversible damage to those cells, such that stabilization of an altered collagen network cannot occur through neocollagenesis. Damage to the living cells of the dermis will negatively affect the ability of the dermis to respond to treatment through the wide variety of healing processes available to the skin tissue. Adjuvants that lower the thermal transition temperature required for shrinkage have the advantage that less total heat need be applied to the target tissue to induce shrinkage, thus limiting the amount of heat accumulating in the next adjacent subcutaneous tissue layer (hypodermis). Reducing the total energy application is expected to minimize tissue damage to the sensitive cells of the hypodermis, thereby limiting damage to the contour determining adipose cells.

One effect of such adjuvants is that certain chosen biocompatible reagents have the effect of lowering the temperature required to begin disruption of certain molecular forces. In essence, adjuvants are capable of reducing the molecular forces stabilizing the ultrastructure of the skin, allowing a lower absolute temperature to induce shrinkage of the collagen network that determines the anatomy of the skin. Any substance that interferes with the molecular forces stabilizing collagen molecules and collagen fibers will exert an influence on the thermal transformation temperature (melting temperature). As collagen molecules melt, the three dimensional structure of collagen undergoes a transition from the triple helix structure to a more random polypeptide coil. The temperature at which collagen shrinkage begins to occur is that point at which the molecular stabilizing forces are overcome by the disruptive forces of thermal transformation. Collagen fibers of the skin stabilized in the ECM by accessory proteins and compounds such as hyaluronan and chondroitin are typically stable up to a temperature of approximately 58° C. to 60° C., with thermal transformation and shrinkage occurring in a relatively narrow phase transition range of 60-70° C. Variations of this transition range are noted to occur in the aged (increasing the transition temperature) and in certain tissues (decreasing by 2-4° C. in tendon collagen). In effect, the lower temperature limit of the collagen shrinkage domain, is determined by the thermal transformation temperature of a particular collagen containing structure.

It will be recognized by those skilled in molecular biology that the thermal transformation temperature necessary to achieve a reduction in skin laxity may not entirely be determined by the thermal transformation temperature of collagen fibers, but may also be affected by a variety of other macromolecules present in the dermis, including other structural proteins such as elastin, fibronectin, heparin, carbohydrates such as hyaluronan and other molecules such as water and ions.

Substances exhibiting the properties desirable for lowering the thermal transition temperature include enzymes such as hyaluronidase, collagenase and lysozyme; compounds that destabilize salt bridges, such as beta-naphtalene sulphuric acid; each of which is expected to reduce the thermal transition temperature by 10-12° C., and substances that interfere with hydrogen bonding and other electrostatic interactions, such as ionic solutions, such as calcium chloride or sodium chloride; detergents (a substance that alters electrostatic interactions between water and other substances), such as sodium dodecyl sulphate, glycerylmonolaurate, cationic surfactants, or N,N, dialkyl alkanolamines (i.e. N,N-diethylethanolamine); lipophilic substances (lipophiles) including steroids, such as dehydroepiandrosterone, and oily substances such as eicosapentanoic acid; organic denaturants, such as urea; denaturing solvents, such as alcohol, ethanol, isopropanol, acetone, ether, dimethylsulfoxide (DMSO) or methylsulfonylmethane; and acidic or basic solutions. The adjuvants that interfere with hydrogen bonding and other electrostatic interactions may reduce the thermal transition temperature by as much as 40° C. depending on the concentration and composition of the substances administered. The extent of effectiveness of a particular adjuvant in use will be dependent on the chemical properties of the adjuvant and the concentration of adjuvant administered to the patient. For enzymatic adjuvants such as hyaluronidase, the thermal transition temperature is also dependent on the specific activity of the delivered enzyme adjuvant in the dermis environment.

Adjuvants suitable for use would desirably be compatible with established medical protocols and be safe for use in human patients. Adjuvants should be capable of rapidly infiltrating the targeted skin tissue, should cause minimal negative side effects, such as causing excess inflammation, and should preferably persist for the duration of the procedure. Suitable adjuvants may be, for instance, combined with local anesthetics used during treatment, be injectable alone or in combination with other reagents, be heat releaseable from the implants of the invention, or be capable of entering the targeted tissue following topical application to the skin surface. Certain large drug molecules, such as enzymes functioning as adjuvants according to the invention may be drawn into the target dermal tissue through iontophoresis (electric current driving charged molecules into the target tissues) The exact mode administration of adjuvants will be dependent on the particular adjuvant employed.

Preferably, the thermal transition temperature lowering adjuvant is present in highest concentrations in the tissues of the dermis. For highest efficacy, a concentration gradient is established, wherein the adjuvant is at a higher concentration in the dermis that in the hypodermis. A transdermal route of administration is one preferred mode of administration, as will occur with certain topical adjuvants. For adjuvants that are applied topically to the surface of the skin, for instance as a pomade, as the adjuvant either diffuses or is driven across the epidermis, and passes into the dermis, a concentration gradient is established wherein the adjuvant concentration is higher in the dermis than in the hypodermis. Because the collagen matrix is much more prevalent in the dermis than in the epidermis, presence of the adjuvant in the epidermis is expected to be without negative effect. Certain adjuvants, for instance, enzymes with collagen binding activity, would be expected to accumulate in the dermal tissue.

A variety of methods are known wherein drugs are delivered to the patient transdermally, i.e. percutaneously, through the outer surface of the skin. A variety of formulations are available that enhance the percutaneous absorption of active agents. These formulations may rely on modification of the active agent, or the vehicle or solvent carrying that agent. Such formulations may include solvents such as methylsulfonylmethane, skin penetration enhancers such as glycerylmonolaurate, cationic surfactants, and N,N, dialkyl alkanolamines such as N,N-diethylethanolamine, steroids, such as dehydroepiandrosterone, and oily substances such as eicosapentanoic acid. For further discussion of enhancers of transdermal delivery of active agents, for instance adjuvants according to the invention, see: U.S. Pat. No. 6,787,152 to Kirby et al., issued Sep. 7, 2004; and U.S. Pat. No. 5,853,755 to Foldvari, issued Dec. 29, 1998.

When adjuvants are injected, it is preferable that they be deposited as close to the dermis as practicable, preferably, intradermally. Because the dermis is relatively thin, and difficult to penetrate with hypodermic needles, the invention is also embodied in adjuvants that are delivered subdermally, or at the interface between the dermis and the next adjacent subcutaneous tissue (hypodermis or adipose tissues underlying the dermis). Even to the extent that adjuvants are delivered into the adipose tissue of the hypodermis, because the hypodermis is typically very thick compared to the dermis, a concentration gradient will develop, wherein the adjuvant will diffuse quickly into the dermis, and fully equilibrate with the dermal tissue, before the adjuvant has fully equilibrated with the hypodermis.

In a further embodiment, the implants carry a surface coating of adjuvant that is released into the dermis upon activation of the implant. It is an advantage of the invention when utilizing thermal transition temperature lowering adjuvants that the implants are placed very near the location where adjuvants can provide the most benefit. A number of compositions are known in the art that can be released from an implant by heating of the implant. For example, the upper, or dermis facing, surface of the implant can be coated with microencapsulated adjuvant, for instance hyaluronan. Once a preliminary heating of the implant begins, the encapsulated adjuvant is released, and immediately begins diffusing into the dermis tissue, as the implant is already in place at the interface between the dermis and hypodermis. As the adjuvant diffuses through the dermis, a concentration gradient develops wherein the adjuvant is at the greatest concentration in the dermis, with reduced concentrations in the epidermis and hypodermis. Following this preliminary heating, regular ramp up to a lowered setpoint temperature may be carried out. As described previously, while it is not a requirement that the adjuvant be at greatest concentration in the dermis (for instance, if the adjuvant is applied topically to the skin surface), it is considered an advantage to for the adjuvant to be at the greatest concentration in the tissue layer wherein adjuvant activity is needed.

In a further embodiment of implant delivery of the adjuvant, the adjuvant is encapsulated in liposomes and suspended in a compatible vehicle. The surfaces of the implant to be inserted into the patient are then coated with the liposome/vehicle composition. When the implant is inserted into the tissue of the patient, the vehicle coating, preferably moderately water soluble and biologically inert, prevents the adjuvant from being displaced from the implant surface for the period of time necessary for insertion. Once the implant is activated on the noted preliminary basis, the dermis facing upper surface of the implant is heated and the liposomes encapsulating the adjuvant are induced by heat to release the adjuvant. The adjuvant may alternatively be released from implants by brief preliminary heating. Different compositions of liposomes are useful for providing release of the adjuvant at a particular temperature range. Similarly, the vehicle binding the adjuvant encapsulating liposomes to the implant can be chosen so that the vehicle does not release the liposomes themselves unless a desired temperature has been reached. In this manner the release of adjuvant from an implant surface may be configured so that the adjuvant is released in a directional manner, even though the entire implant surface is coated with an adjuvant composition. Those skilled in the art will recognize that a variety of heat releaseable encapsulating systems are available for use with the invention. Further discourse on the composition of liposomes is available by referring to U.S. Pat. No. 5,853,755 (supra).

The following discourse specifically describes certain embodiments of specific adjuvants that are useful. Artisans will recognize that other substances known in the art to have similar effects will be useful as adjuvants, and thus, the following embodiments should not be considered as limiting.

Hyaluronidase is an enzyme that cleaves glycosidic bonds of hyaluronan, depolymerizing it and, converting highly viscous polymerized hyaluronan into a watery fluid. A similar effect is reported on other acid mucopolysaccharides, such as chrondroitin sulphate. Hyaluronidase is commercially available from a number of suppliers (e.g., Hyalase, C.P. Pharmaceuticals, Red Willow Rd. Wrexham, Clwydd, U.K.; Hylenex, Halozyme Therapeutics (human recombinant form); Vitrase, (purified ovine tissue derived form) ISTA Pharmaceuticals; Amphadase, Amphastar Pharmeceuticals (purified bovine tissue derived)).

Hyaluronidase modifies the permeability of connective tissue following hydrolysis of hyaluronan. As one of the principal viscous polysaccharides of connective tissue and skin, hyaluronan in gel form, is one of the chief ingredients of the tissue cement, offering resistance to the diffusion of liquids through tissue. One effect of hyaluronidase is to increase the rate of diffusion of small molecules through the ECM, and presumably to decrease the melting temperature of collagen fibers necessary to induce shrinkage. Hyaluronidase has a similar lytic effect on related molecules such as chondroitin sulphate. Hyaluronidase enhances the diffusion of substances injected subcutaneously, provided local interstitial pressure is adequate to provide the necessary mechanical impulse. The rate of diffusion of injected substances is generally proportionate to the dose of hyaluronidase administered, and the extent of diffusion is generally proportionate to the volume of solution administered. The addition of hyaluronidase to a collagen shrinkage protocol results in a reduction of the thermal transition temperature required to induce 20% collagen shrinkage by about 12° C. Review of pharmacological literature reveals that doses of hyaluronidase in the range of 50-1500 units are used in the treatment of hematomas and tissue edema. Thus, local injection of 1500 IU hyaluronidase in 10 ml vehicle into the target tissue is predicted to reduce the temperature necessary to accomplish 20% shrinkage of collagen length from about 63° C. to about 53° C. For multiple injection sites 100 IU hyaluronidase in 2 ml of alkalinized normal saline or 200 IU/ml are expected to be similarly effective as an adjuvant. The manufacturer's recommendations for Vitrase indicate that 50-300 IU of Vitrase per injection are expected to exert the adjuvant effect. It should be noted that use of saline vehicle for delivery of adjuvants and anesthesia may be contraindicated where introduction of excess electrolytes would interfere with operation of the implants.

Hyaluronidase has been used in clinical settings as an adjunct to local anesthesia for many years, without significant negative side effects, and is thus believed to be readily adaptable for use with the instant method. When used as an adjunct to local anesthesia, 150 IU of hyaluronidase are mixed with a 50 ml volume of vehicle that includes the local anesthetic. A similar quantity of hyaluronidase is expected to be effective for reducing the thermal transition temperature for effecting shrinkage by approximately 10° C., with or without the addition of anesthetic. When hyauronidase is injected intradermally or peridermally, the dermal barrier removed by hyaluronidase activity persists in adult humans for at least 24 hours, with the permeabilization of the dermal tissue being inversely related to the dosage of enzyme delivered (in the range of administered doses of 20, 2, 0.2, 0.02, and 0.002 units per mL. The dermis is predicted to be restored in all treated areas 48 hours after hyaluronidase administration. Additional background on the activity of hyaluronidase is available by referring to the following publications (and the references cited therein):
28. Lewis-Smith, P. A., “Adjunctive use of hyaluronidase in local anesthesia” Brit. J. Plastic Surgery, 39: 554-558 (1986).
29. Clark, L. E., and Mellette, J. R., “The Use of Hyaluronidase as an Adjunct to Surgical Procedures” J. Dermatol., Surg. Oncol., 20: 842-844 (1994).
30. Nathan, N., et al., “The Role of Hyaluronidase on Lidocaine and Bupivacaine Pharmaco Kinetics After Peribulbar Blockade” Anesth Analg., 82: 1060-1064 (1996).

See also U.S. Pat. No. 6,193,963 to Stern, et al., issued Feb. 27, 2001.

Lysozyme is an enzyme capable of reducing the cementing action of ECM compounds such as chondroitin sulphate. Lysozyme (aka muramidase hydrochloride) has the advantage that it is a naturally occurring enzyme; relatively small in size (14 kD), allowing rapid movement through the ECM; and is typically well tolerated by human patients. A topical preparation of lysozyme, as a pomade of lysozyme is available (Murazyme, Asta Medica, Brazil; Murazyme, Grunenthal, Belgium, Biotene with calcium, Laclede, U.S.). The addition of lysozyme as an adjuvant to a collagen shrinkage protocol results in a reduction of the thermal transition temperature required to induce 20% collagen shrinkage by about 10-12° C. Additional background on the use of lysozyme to lower the thermal transition temperature for collagen shrinkage is available. See for instance, U.S. Pat. No. 5,484,432 to Sand, issued Jan. 16, 1996.

Those skilled in the art will recognize that a variety of adjuvants that reduce the stability of the collagen fiber, tropocollagen, and or substances that serve to cement these structures are adaptable for use with the heater implants of the invention. Adjuvant ingredients may include agents such as solvents, such as dimethylsulfoxide (DMSO), monomethylsulfoxide, polymethylsulfonate (PMSF), methylsulfonylmethane, alcohol, ethanol, ether, diethylether, and propylene glycol. Certain solvents, such as DMSO, are known to lead to the disruption of collagen fibers, and collagen turnover. When DMSO is delivered to patients with scleroderma, a condition that exhibits an overproduction of collagen and scar tissue as a symptom, an increase of excretion of hydroxyproline, a constituent of collagen, is noted. This is believed to due to increased breakdown of collagen. Solvents that will alter the hydrogen bonding interactions of collagen fibers, such as DMSO and ethanol are predicted to reduce the thermal transition temperature necessary to reach the thermal transition temperature of collagen fibers, with the reduction of thermal transition temperature being expected to be relative to the alteration of the hydrophilicity of the collagen environment by the solvent. Small diffusible solvents such as DMSO and ethanol offer the further advantage of being able to rapidly penetrate the epidermis and reach the dermis tissue, while being generally safe for use in human patients.

In a further embodiment, adjuvants may be used in combination with one another, in a manner that either further lowers the thermal transition temperature either synergistically or additively. Combining adjuvants provides a means to utilize a particular adjuvant to achieve its optimal effect, and when combined with a second adjuvant, further lower the heating necessary to achieve the desired shrinkage, while avoiding adverse side effects associated with higher doses of a particular adjuvant.

The practitioner may also wish to consider the utilization of a local anesthetic having generally electrically neutral or non-conductive diluent. However, where the above-discussed blocking and remotely administered local anesthetic is utilized, the possible adverse effect of isotonic saline diluent is not present.

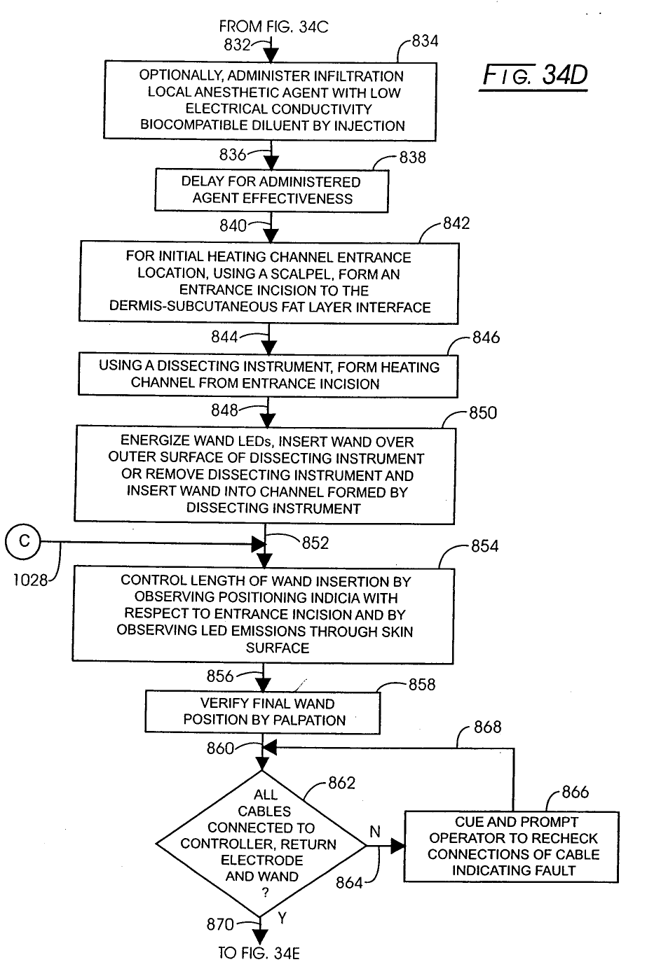

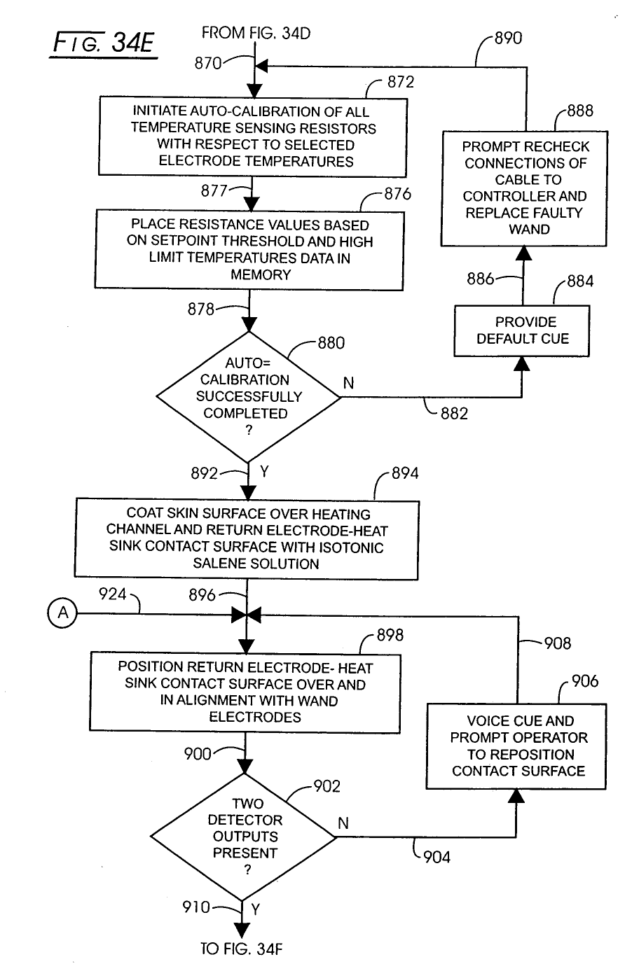

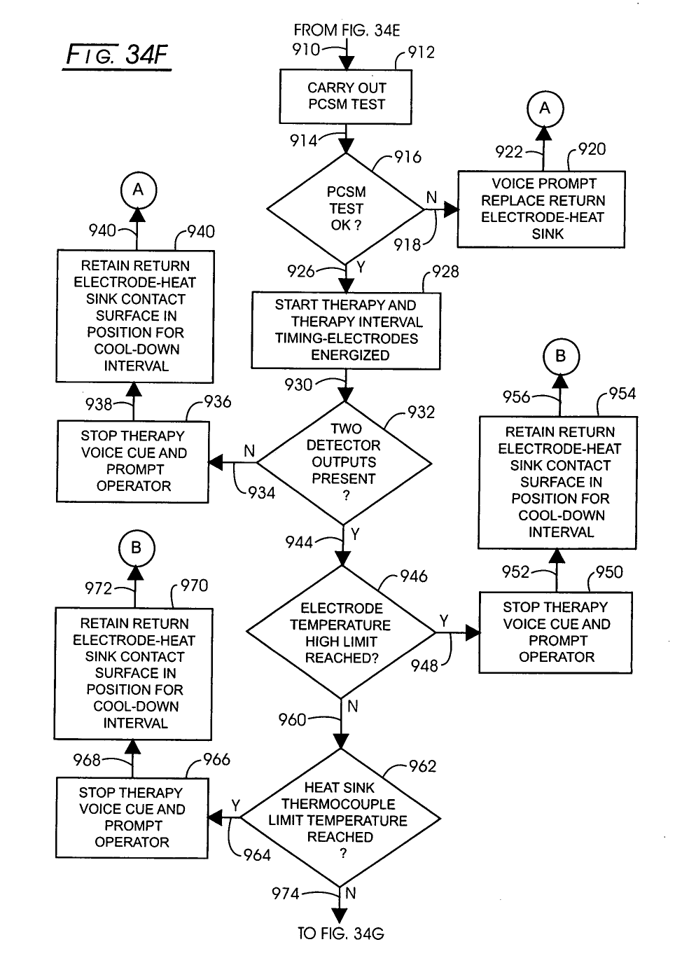

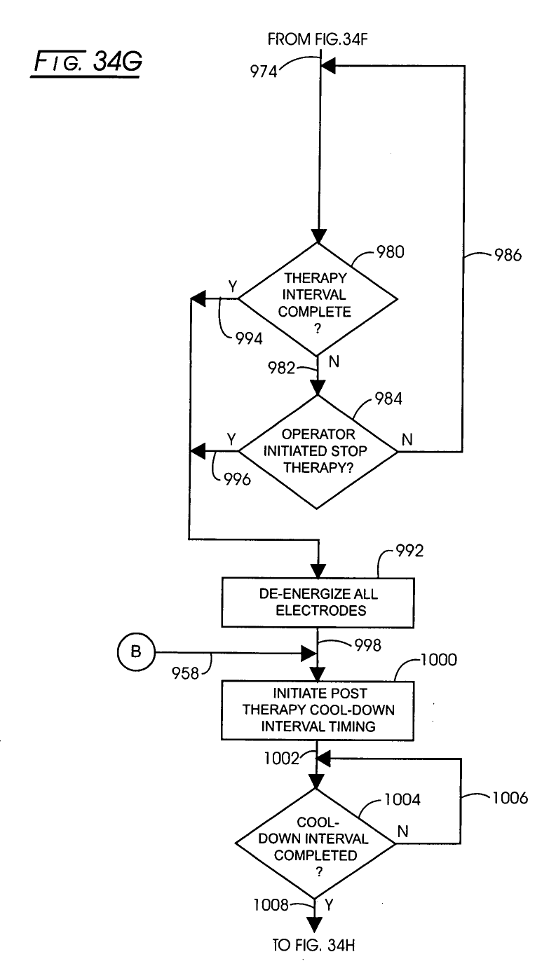

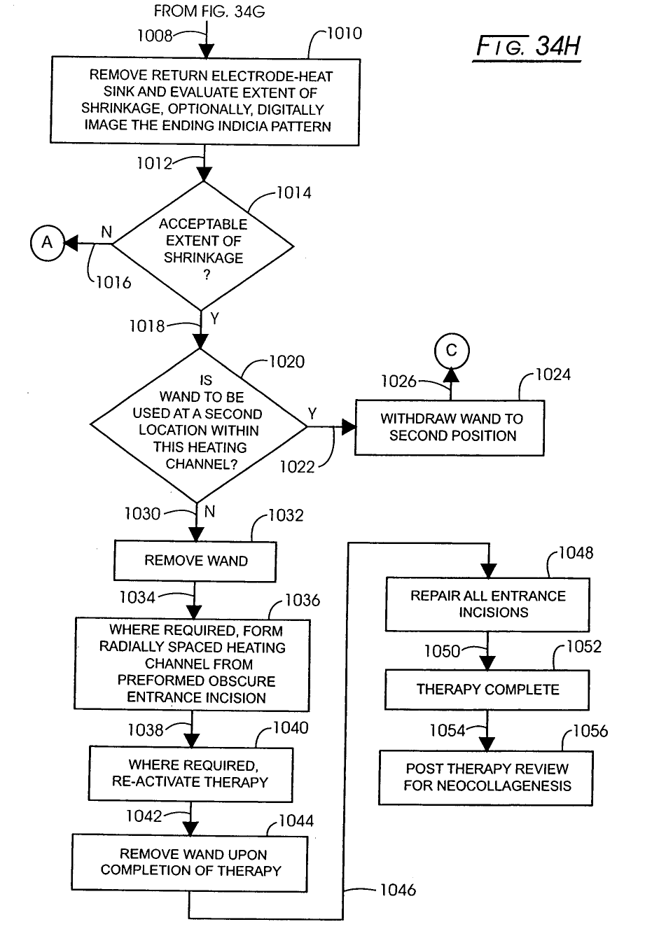

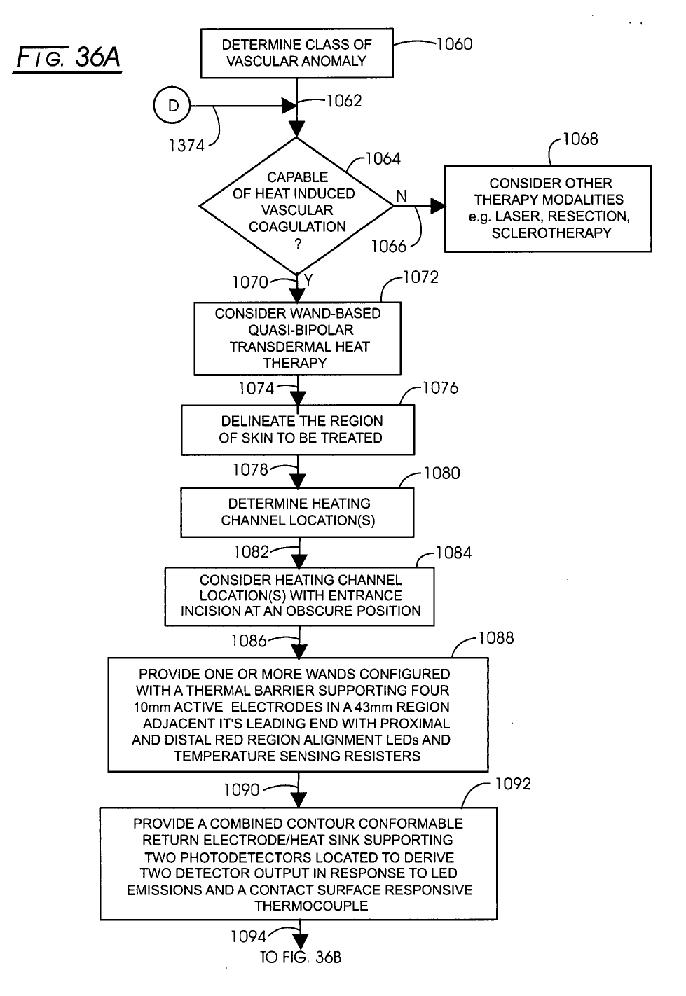

FIGS. 34A-34H combine as labeled there on to provide a flowchart describing the method and system of the teachings at hand. Referring to FIG. 34A, as represented at block 720, the practitioner determines the skin region elected for contraction. Next, as represented at line 722 and block 724, the practitioner estimates the desire extent of linear shrinkage. In general, this election will be at the level of about 10%, however, the shrinkage should generally not exceed about 25%. As represented at line 726 and block 728, the practitioner then determines heating channel location(s) considering a parallel relationship with Langer's (relaxed intrinsic skin tissue) lines. As discussed in the background hereof, in the course of carrying out animal (pig) studies, and considering journal articles concerning Langer's lines in conjunction with animal studies, the practitioner may find it appropriate to insert the implant or thermal wand in parallel relationship with what may be considered Langer's lines. In FIG. 35, a human head is represented in general at 730 along with dashed line representations of one publication derived representation of these intrinsic skin tension lines as shown, for instance at 731 and 732. Note in the figure that a thermal wand or implant 734 as described above is located within a heating channel, which is generally parallel with lines of intrinsic skin tension, such as line 732. When inserting implant 734 parallel to Langer's lines, the amount of relative shrinkage is expected to be enhanced relative to an insertion orientation perpendicular to Langer's lines, i.e. parallel to lines of relaxed intrinsic skin tension. Tension lines and lines of relaxed intrinsic skin tension are also present at other locations on the body, such as line 736 in FIG. 35.

Returning to FIG. 34A, a line 738 is seen extending to block 740 wherein the practitioner considers heating channel location(s) with an entrance location at an obscure position, for example, adjacent an ear, such selection being made for cosmetic purposes. Next, as represented at line 742 and block 744, one or more thermal wands are provided. In this regard, typically only one wand is employed at a time inasmuch as the return electrode and heat sink function is a hand-held one. Such wands, for example, have been described in connection with FIGS. 4-7. Note, additionally, that proximal and distal red region alignment LEDs and temperature sensing resistors are incorporated with these wands. From block 744, line 746 extends to block 748 wherein the combined contour conformable return electrode and heat sink is provided as described in connection with FIGS. 24 and 25. The device may also support two photo detectors located to derive two detector outputs in response to LED missions. Additionally, the device may incorporate a contact surface responsive thermocouple. Recall additionally, as discussed in connection with FIG. 26, this return electrode and heat sink function may be implemented with a solid block of conductive material such as aluminum. As represented at line 750 and block 752, one or more introducer instruments as described in connection with FIGS. 21 and 22 are provided for the dissection of a heating channel or channels. From block 752, line 754 extends to block 756 and provides a controller, for energization and control over the active electrodes and return electrode, resistor segments, LEDs, thermocouples, photo-detectors and PCSM circuit. Such a controller has been described in connection with FIGS. 27-29. Next, as represented at line 758 and block 760, the operator selects a high limit and setpoint threshold temperature for the electrodes as discussed in connection with FIG. 33. Optionally, a lower limit temperature may be set. Additionally, as represented at line 762 and block 764, a limit temperature is set for the heat sink thermocouple. Next, as represented at line 766 and block 768, a query is posed as to whether or not an adjuvant is to be used. In the event that it is to be used, then as represented at line 770 and block 772, a determination is made as to which adjuvant is to be employed. Next, as represented at line 774 and block 776, there will be an adjustment to the electrode setpoint threshold level and the high limit temperature as discussed respectively at lines 678 and 680 in FIG. 33. For example, these temperature levels may be reduced by ΔTₐ. As represented at line 778 and block 780, the elected adjuvant is administered at the skin region for shrinkage and as represented at line 782 and block 784, a delay ensues for an interval effective to diffuse the adjuvant. The procedure then proceeds as represented at line 786. Returning to block 768, where an adjuvant is not to be use, then the procedure continues as represented at line 788 which extends to block 790 as does line 786. Block 790 provides for optionally providing a starting pattern of visible indicia at the skin region suited for evaluating a percentage of shrinkage. Such an indicia has been illustrated in connection with the animal study associated with FIG. 31. Correspondingly, as represented at line 792 and block 794, if such a pattern is utilized, it may be digitally imaged to provide a pattern reference. Next, as represented at line 796 and block 798, an overall treatment interval may be selected, for example, including a ramp-up interval as well as the estimated interval for N stages as described in conjunction with FIG. 33. Additionally, as represented at line 800 and block 802, a post-therapy cool-down interval is selected. Typically, this interval will be about thirty seconds and during such interval, the practitioner may be voice cued to retain the heat sink function in place. Line 804 extends from 802 to block 806. Block 806 provides for selecting the on-time or power-on interval about the setpoint threshold temperature. As described in connection with FIG. 33, this interval for full power is initially established, for example, in connection with stage 1. Next, as represented at line 808 and block 810, the off-time or power-off interval for the pulses within a stage is selected. Stage timing then is considered as represented at line 812 and block 814. Block 814 provides for selecting the stage interval as discussed in connection with FIG. 33. Next, as represented at line 816 and block 818, the cool-down interval between stages as discussed in connection with FIG. 23 is selected, or established at the lower limit temperature. The program continues as represented at line 820 and block 822 wherein a circuit continuity test is carried out with respect to system components.

A local anesthetic then may be administered as represented at line 824 and block 826. Block 826 indicates that such an anesthetic may be of a conventional type, which contains an isotonic saline diluents. However, as represented at line 828 and block 830, to protect the fat layer and potentially the muscle underlying it, a nerve block removed from the skin region of interest may be administered, again, with a conventional local anesthetic having an isotonic saline diluent. As a further option, as represented at line 832 and block 834, an infiltration local anesthetic agent with a low electrical conductivity biocompatible diluent may be injected. With such an arrangement, current conduction toward the fat layer and muscle underlying it is not enhanced. Following administration of the local anesthetic, as represented at line 836 and block 838, a delay ensues to assure administered agents effectiveness. Following such delay as represented at line 840 and block 842, for an initial heating channel entrance location, the practitioner, using a scalpel forms an entrance incision to the dermis-subcutaneous fat layer interface. Then, as represented at line 844 and block 846, using a dissecting instrument as provided at block 752, the practitioner forms a heating channel from the noted entrance incision. For positioning purposes, as represented at line 848 and block 850, the physician inserts a thermal wand over the outer surface of the dissecting or introducer instrument. Optionally, that dissecting or introducer instrument may be removed from the heating channel and then the thermal wand is inserted. During this insertion, as represented at line 852 and block 854, the length of wand insertion is controlled by observing the positioning indicia with respect to the entrance incision. Such indicia has been identified and illustrated in FIG. 4 at 42 and in FIG. 6 at 62. Additionally, the practitioner may observe the red spectrum transmission from the proximal and distal LEDs as such emissions pass through the dermis and epidermis. Typically, as represented at line 856 and block 858, the practitioner will verify the final wand position within a heating channel by palpation.

A sequence of tests are then carried out, in this regard, as represented at line 860 and block 862, a determination is made as to whether all cables are connected to the controller, return electrode and wands. In the event they are not, as represented at line 864 and block 866, the operator is cued and prompted to recheck the connections of any cable indicating a fault. The program loops then as represented at line 868 to line 860. Where all cables are appropriately connected, the program continues as represented at line 870 extending to block 872. That block provides for the initiation of auto-calibration of all temperature sensing resistors with respect to selected electrode temperatures (see equation (3) above). As represented at line 874 and block 876, resultantly derived resistance values based on setpoint threshold temperature, high limit temperature, and optionally lower limit temperature are placed in memory. In this regard, reference is made again to FIG. 33. Next, as represented at line 878 and block 880, a determination is made as to whether the auto-calibration has been successfully completed. In the event that it has not, then as represented at line 882 and block 884, a fault cue is promulgated to the operator and as represented at line 886 and block 888, the operator is prompted to recheck the connections of cable to the controller and replace a faulty wand, whereupon the program loops to line 870 as represented at line 890.

Returning to block 880, where auto-calibration has successfully been completed, then as represented at line 892 and block 894 the skin surface over the heating channel and the return electrode-heat sink contact surface are coated with a thermally and electrically conductive solution such as isotonic saline solution. Then, as represented at line 896 and block 898, the operator positions the return electrode-heat sink contact surface over and in alignment with the four wand electrodes. A check as to proper alignment then is carried out. In this regard, line 900 extends to the query posed at block 902 determining whether the photo-detectors within the return electrode-heat sink have provided two detector outputs. Where two such outputs are not present, then as represented at line 904 and block 906 the controller promulgates a voice cue and prompt to the operator to reposition the contact surface. Utilization of a voice cue for this purpose is helpful inasmuch as the practitioner is in the process of holding the heat sink over the four electrodes of the wand. The program then loops as represented at line 908 to line 896. Where two detector outputs are present, then as represented at line 910 and block 912 a patient circuit safety monitor (PCSM) test may be carried out and, as represented at line 914 and block 916, a determination is made as to whether the PCSM test was ok. In the event that it was not, then as represented at line 918 and block 920, the practitioner is voice prompted to replace the return electrode-heat sink. The program then loops as represented at line 922 and node A. Node A reappears in conjunction with line 924 extending to line 896. Where the PCSM test is found to be ok, then as represented at line 926 and blank 928, therapy is started and the therapy interval timing commences. The active electrodes are energized and the temperature sensing resistor segments are enabled. During therapy, as represented at line 930 and block 932, a check is made to ascertain that the two photo-detector detector outputs are present. Where they are not, as represented at line 934 and block 936, therapy is stopped and a voice cue and prompt is provided to the operator. In this regard, as represented at line 938 and block 940, this voice prompt instructs the practitioner to retain the return electrode-heat sink contact surface in position for a cool-down interval. This is to avoid any epidermal burn or thermal trauma. The program then continues as represented at line 942 to earlier-described node A and line 896.

Returning to block 832, where two detector outputs remain present, then as represented at line 944 and block 946, a determination is made as to whether the electrode temperature high limit has been reached. This high limit temperature has been described in FIG. 33 in conjunction with dashed line 680. Where that high limit temperature has been reached, then as represented at line 948 and block 950, therapy is stopped and the practitioner is given a voice cue and prompt. In that regard, as represented at line 952 and block 954, the prompt to the operator is to retain the return electrode-heat sink contact surface in position for a cool-down interval. The program then continues as represented at line 956, which extends to node B. Node B reappears in conjunction with line 958 extending to line 998. Where the high limit temperature has not been reached, then as represented at line 960 and block 962, a determination is made as to whether the heat sink thermocouple limit temperature has been reached. In the event that it has, then as represented at line 964 and block 966, therapy is stopped and a voice cue and prompt is provided to the practitioner. As represented at line 968 and block 970, the voice prompt tells the operator to retain the return electrode-heat sink contact surface in position for a cool-down interval. The procedure then continues to earlier-described node B as represented at line 972.

Where the thermocouple limit temperature has not been reached, then as represented at line 978 and block 980, a determination is made as to whether the therapy interval is completed. In the event that it is not completed, as represented at line 982 and block 984, a determination is made as to whether an operator initiated stop therapy is in place. In the event that it has not been initiated, then the program loops as represented at line 986 to line 974.

Returning to block 980, where the therapy interval is completed, then as represented by lines 994, 990 and block 992, all electrodes are de-energized. Finally, where the operator has initiated a stop therapy, as represented at line 996, line 990 and block 992, all electrodes are de-energized. With such de-energization, as represented at line 998 and block 1000, the post therapy cool-down interval timing is initiated. Typically, this interval will be about thirty seconds. With the initiation of the cool-down interval, as represented at line 1002 and block 1004, a determination is made as to whether the cool-down interval has been completed. In the event that it has not, then the program loops as represented at line 1006 to line 1002. Where the post therapy cool-down interval is completed, then as represented at line 1008 and block 1010, the practitioner removes the return electrode-heat sink from contact over the thermal wand and evaluates the extent of shrinkage. Optionally, the ending indicia pattern may be digitally imaged such that average lineal shrinkage can be computed. The program continues as represented at line 1012 and block 1014 determining whether an acceptable extent of shrinkage has been reached. In the event it has not been reached, then as represented at line 1016 the program reverts to node A. As noted above, node A reappears in conjunction with line 924 extending to line 896. Where an acceptable extent of shrinkage has been reached, then as represented at line 1018 and block 1020 a determination is made, as to whether the thermal wand is to be used at a second location within the present heating channel. Where it is to be so used a second time, then as represented at line 1022 and block 1024, the wand is withdrawn to an extent that the four electrodes thereof are at the desired second position. The program then continues as represented at line 1026 and node C. Node C reappears with line 1028 extending to line 852.

Returning to block 1020, where the wand is not to be utilized at a second location within the instant heating channel, then as represented by line 1030 and block 1032, the wand is removed from this heating channel. The program continues as represented at line 1034 and block 1036, which provides that where required, a radially spaced heating channel may be provided which extends from a preformed obscure entrance incision. Where such a requirement exists, as represented at line 1038 and block 1040, the therapy may be reactivated with respect to this newly formed heating channel as represented at line 1042 and block 1044. At the completion of such reactivated therapy, the wand is removed and as represented at line 1046 and block 1048 all entrance incisions are repaired. As represented at line 1050 and block 1052, therapy is completed. Upon completion of the therapy, as represented at line 1054 and block 1056, there is provided a post therapy review to determine whether appropriate neocollagenisis has occurred.

The present system can also be utilized to treat certain vascular anomalies as are discussed in the Background, and elsewhere in the disclosure. As noted in that Background, attempts have been made classify these vascular anomalies with sub-classifications having been developed, for example, with respect to port wine stains.

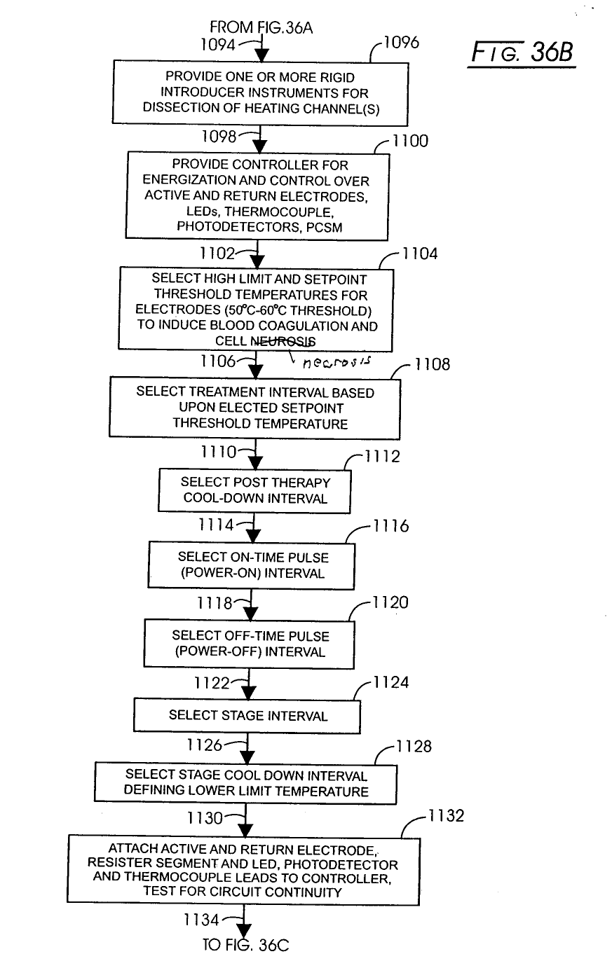

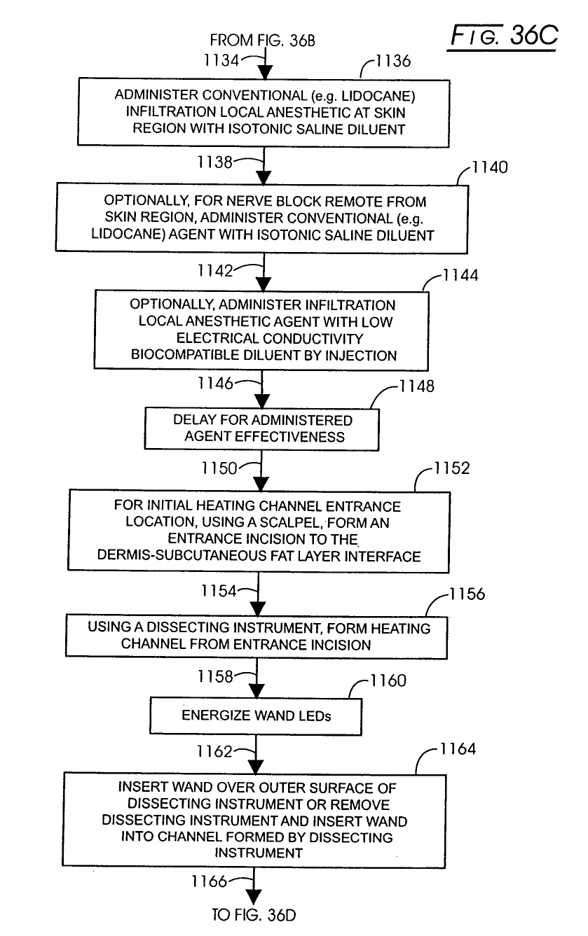

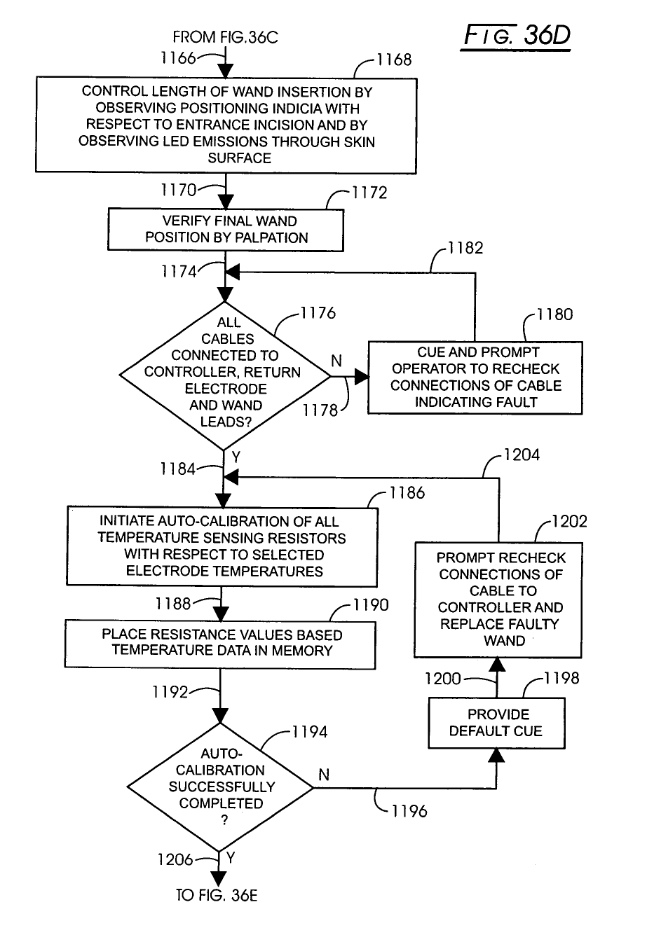

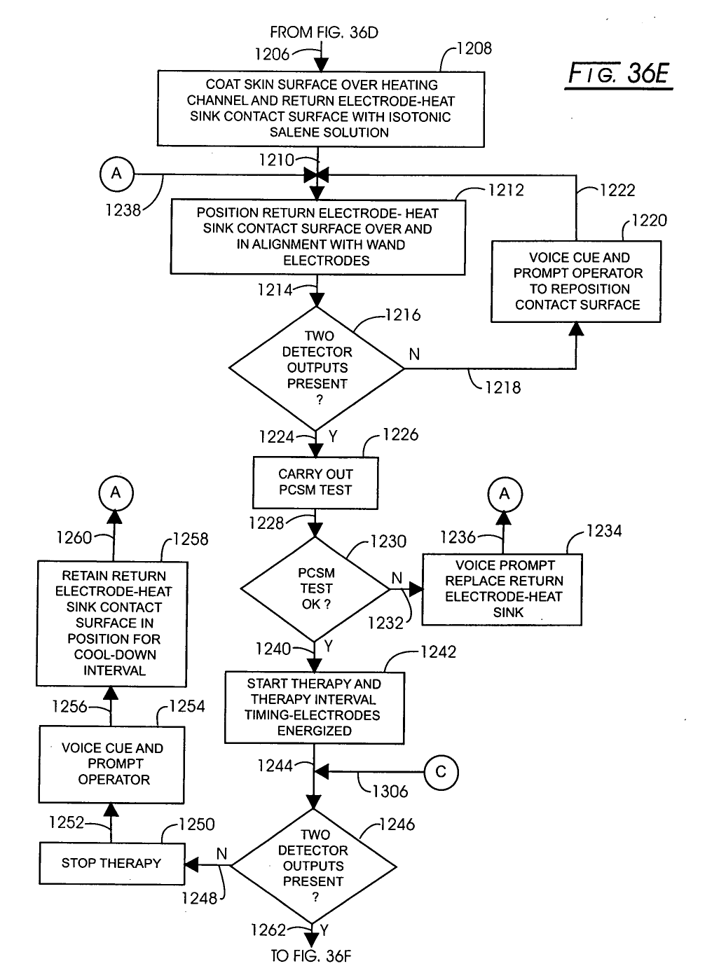

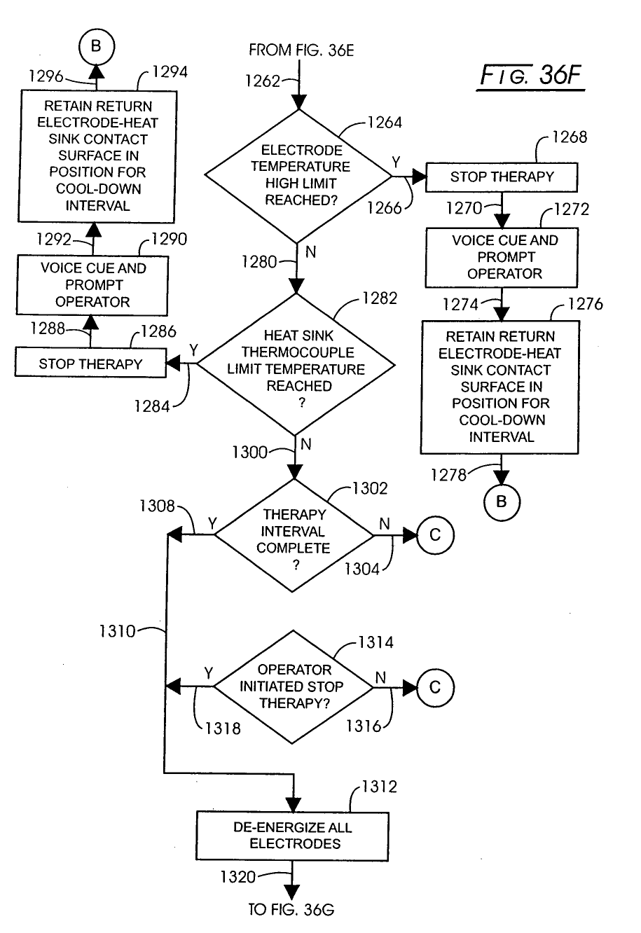

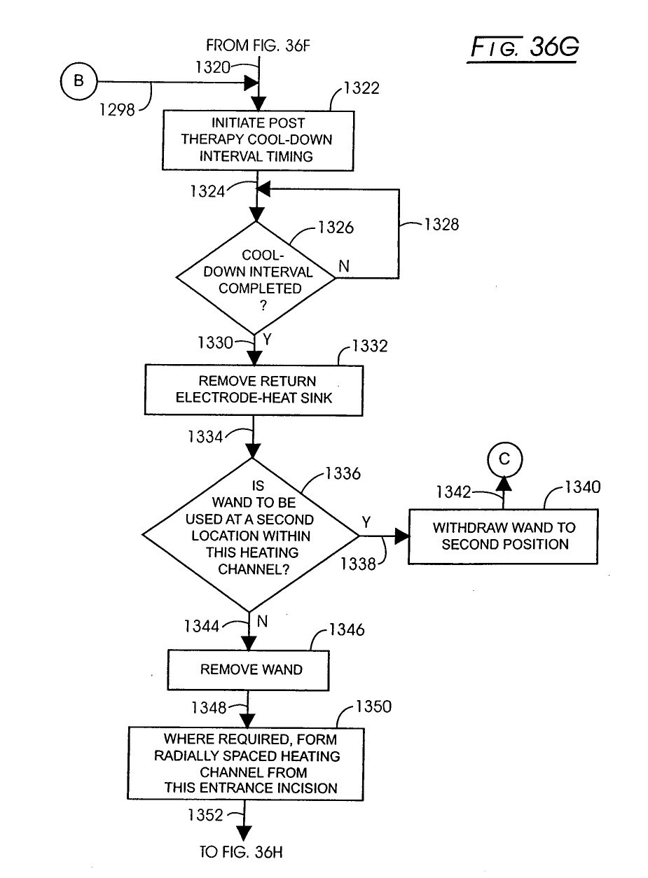

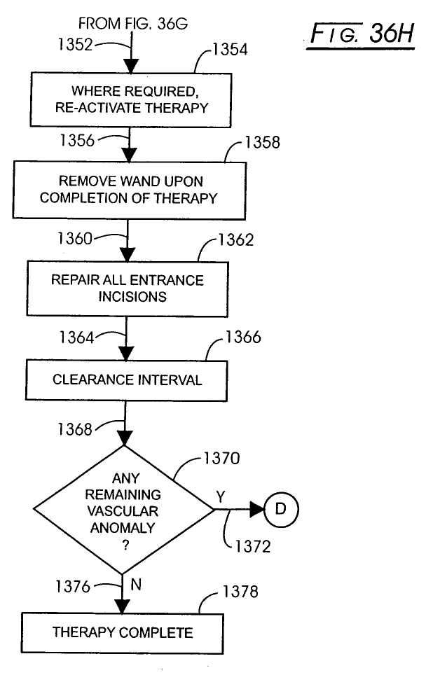

FIGS. 36A-36H should be considered together as labeled thereon. Reference is made to FIG. 36A. As represented at block 1060 an initial determination is made as to the class vascular anomaly at hand. Such determination involves a decision as is represented at line 1062 and block 1064. That decision is the subject as to whether the identified class is capable of heat-induced vascular coagulation. If it is not, then as represented at line 1066 and block 1068, a consideration is made as to other therapy modalities, for example, laser therapy, resection or sclerotherapy. Where an affirmative determination is made with respect to the query at block 1064, then as represented at line 1070 and block 1072, the instant system should be considered which is a wand-based, quasi-bipolar transdermal heat therapy. Then, as represented at line 1074 and block 1076, a region of skin to be treated is delineated and, as represented at line 1078 and block 1080, a determination is made as to the location of a heating channel or channels. Since there is no consideration of dermis shrinkage, the earlier-discussed Langer line evaluation need not be made. As represented at line 1082 and block 1084, the practitioner may consider heating channel location or locations with an entrance incision located at an obscure position, for example, near the ear. Next, as represented at line 1086 and block 1088, one or more wands are provided as has been described in connection with FIGS. 4 and 6. Those wands will incorporate four active electrodes, four corresponding temperature sensing resistor segments and proximal and distal LEDs, which emit in the red region of the spectrum. Additionally, as represented at line 1090 and block 1092, a combined return electrode-heat sink incorporating two photo-detectors located to derive two detector outputs in response to the LED emissions as well as having a contact surface responsive thermocouple. This heat sink may be contour conformable as described in connection with FIGS. 24 and 25 or a solid block as described in connection with FIG. 26. Line 1094 extends from block 1092 to block 1096 providing for the provision of one or more rigid introducer instruments for dissection of a heating channel or channels. Such an instrument has been described in connection with FIGS. 21 and 22. Line 1098 extends from block 1096 to block 1100, describing the provisional controller for energization and control over the active and turn electrodes, LEDs, thermocouple, photo-detectors and PCSM. With that controller, as represented at line 1102 and block 1104, the high limit and setpoint threshold temperatures for the electrode are selected to induce blood coagulation and cell necrosis. This setpoint threshold generally will be within a range from about 50° C. to about 60° C. As represented at line 1106 and block 1108, a treatment interval is selected based upon the elected setpoint threshold temperature. Next, as represented at line 1110 and block 1112, the post therapy cool-down interval is selected. During this interval the four electrodes are de-energized and the heat sink function is maintained. As represented at line 1114 and block 1116 the on-time or pulse power-on interval is selected. As discussed in connection with FIG. 33 this interval will typically occur at stage 1 and subsequent to the ramping-up pulse for later stages through stage N. Next, as represented at line 1118 and block 1120 the off-time pulse power-off time interval is selected, following which, as represented at line 1122 and block 1124, the time interval of a stage is selected and, as represented at line 1126 and block 1128 the inter-stage cool-down interval is selected and the program continues as represented at line 1130 to block 1132. Block 1132 provides for attaching the active and return electrodes, resistor segments and LED, photo-detector and thermocouple leads to the controller, whereupon a test for circuit continuity is carried out. Where that test fails, then the operator is prompted to correct the problem and the test is repeated. The program continues as represented at line 1134 and block 1136, which provides for the administration of a conventional infiltration anesthetic on the skin region of interest. Such an agent will contain an isotonic saline diluent. As represented at line 1138 and block 1140, a conventional anesthetic agent may be employed for a nerve block located remotely from the skin region of interest. As another option, as represented at line 1142 and block 1144, the administered anesthetic agent may be provided with a low electrical conductivity biocompatible diluent. As represented at line 1146 and block 1148, a delay ensues for the administered agent to become effective. Then, as represented at line 1150 and block 1152, for the initial heating channel entrance location, using a scalpel, an entrance incision is formed to the dermis-subcutaneous fat layer interface. With the formation of that entrance incision, as represented at line 1154 and block 1156, using a dissecting instrument as described at block 1096, a heating channel is formed from the entrance incision, whereupon as represented at line 1158 and block 1160, the proximal and distal LEDs are energized. With such energization as represented at line 1162 and block 1164 a thermal wand is inserted over the upper surface of the dissecting instrument. Optionally, the dissecting instrument can be removed from the heating channel and a thermal wand then is inserted into the thus-formed channel. In the process of wand insertion, as represented at line 1166 and block 1168, the practitioner may control the extent or length of wand insertion by observing the positioning indicia with respect to the entrance incision. Such positioning indicia, has been identified at 42 in FIG. 4 and at 62 in FIG. 6. Electrode position can also be observed by observing the LED emissions through the skin surface. Next, as represented at line 1170 and block 1172, thermal wand position can be verified by palpation. With the wand thus positioned, as represented at line 1174 and block 1176, a determination is made as to whether all cables are appropriately connected between the controller, return electrode and wand leads. In the event they are not, then as represented at line 1178 and block 1180, the controller cues and prompts the practitioner or operator to recheck the connections of the cable indicating fault and as represented at line 1182 the program loops to line 1174. Where the cables are appropriately connected then, as represented at line 1184 and block 1186, the program initiates auto-calibration of all temperature sensing resistors with respect to the selected electrode temperatures. With the corresponding development of resistor values, as represented at line 1188 and block 1190, the resultant resistance value based temperature data is placed in memory and the procedure continues as represented at line 1192 to the query posed at block 1194. At block 1194 determination is made as to whether the auto-calibration has been successfully completed. If it has not, then as represented at line 1196 and block 1198 the system provides a fault cue and as represented at line 1200 and block 1202, the practitioner is prompted to recheck connections of the cables to the controller and replace a faulty wand, whereupon as represented at line 1204 the program loops to line 1184.

Returning to block 1194, where auto-calibration has been successfully completed, then as represented at line 1206 and block 1208, the skin surface over the heating channel and the return electrode-heat sink contact surface are coated with an isotonic saline solution, which is both thermally and electrically conductive. Upon providing such coatings as represented at line 1210 and block 1212, the return electrode-heat sink contact surface is positioned over and in alignment with the want electrodes. Recall that the two red region emitting LEDs are energized. Accordingly, as represented at line 1214 and block 1216 the controller determines whether two detector outputs from the photo-detectors mounted at the return electrode-heat sink contact surface are present. If both are not present, then as represented at line 1218 and block 1220 a voice cue and voice activated prompt is provided to the operator advising to reposition the contact surface to attain proper alignment. Voice cueing and prompting is utilized here inasmuch as the practitioner or operator will be hand-holding a heat sink over the four electrodes of a thermal wand. The program then loops as represented at line 1222 to line 1210. Where two detectors are present, then as represented at line 1224 and block 1226 the controller carries out a PCSM test, then, as represented at line 1228 and block 1230 a determination is made as to whether the PCSM test has been passed. In the event it has not, then as represented at line 1232 and block 1234, a voice prompt is provided instructing the operator or practitioner to replace the return electrode-heat sink and the program reverts to node A as represented at line 1236. Node A reappears in conjunction with line 1238 extending to line 1210.

Returning to block 1230, where the PCSM test is ok, then as represented at line 1240 and block 1242, therapy is started, the four electrodes are energized and therapy timing commences. As this occurs, the practitioner or operator will be hand-holding the return electrode-heat sink over the four electrodes of the implant. Accordingly, as represented at line 1244 and block 1246, a determination again is made as to whether two detector outputs are present. As noted above, these detector outputs are derived from the photo-detectors mounted at the contact surface of the return electrode-heat sink. Where both of those outputs are not present, then as represented at line 1248 and block 1250, therapy is stopped and, as represented at line 1252 and block 1254, a voice cue and prompt is made to the operator, in particular, as represented at line 1256 and block 1258 the operator prompt is one advising to retain the return electrode-heat sink contact surface in position over the electrodes for a cool-down interval, whereupon the program reverts to node A as represented at line 1260.

Returning to block 1246, where the two detector outputs are present, then as represented at line 1262 and block 1264, a determination is made as to whether the electrode temperature high limit has been reached. In the event that limit has been reached, then as represented at line 1266 and block 1268, therapy is stopped. With such stoppage, as represented at line 1270 and block 1272, the operator or practitioner is voice cued and prompted. In this regard, as represented at line 1274 and block 1276 the voice prompt will provide that the return electrode-heat sink contact surface must be retained in position for a cool-down interval. Next, as represented at line 1278, the program diverts to node B.

Returning to block 1264, where the high limit temperature has not been reached, then as represented at line 1280 and block 1282 a determination is made as to whether the heat sink thermocouple limit temperature has been reached. In the event that it has been reached, then as represented at line 1284 and block 1286, therapy is stopped and as represented at line 1288 and block 1290, a voice cue and prompt is provided to the operator. In this regard, as represented at line 1292 and block 1294 the voice cue and prompt tells the operator or practitioner to retain the return electrode-heat sink contact surface in position for a cool-down interval. As represented at line 1296 the program reverts to node B. Node B reappears in conjunction with line 1298 extending to line 1320.

Returning to block 1282 where the heat sink thermocouple limit temperature has not been reached, then the procedure continues as represented at line 1300 and block 1302. Block 1302 queries as to whether the therapy interval is now completed. If it is not, then as represented at line 1304, the program reverts to node C. Node C reappears in conjunction with line 1306 extending to line 1244. Returning to block 1302, where the therapy interval is completed, then as represented at lines 1308, 1310 and block 1312, all electrodes are de-energized.

Looking to block 1314, a determination made as to whether an operator initiated stop therapy signal has been received. In the event that it has not, then as represented at line 1316, the program reverts to node C. Where an operator initiated stop therapy has occurred, then as represented at lines 1318, 1310 and block 1312, all electrodes are de-energized. Next, as represented at line 1320 and block 1322, post therapy cool-down interval timing is undertaken. With the initiation of cool-down interval timing, as represented at line 1324 and block 1326, a determination is made as to whether the cool-down interval has been completed. If it has not, then as represented at line 1328 the program loops to line 1324. Where the cool-down interval is completed, then as represented at line 1330 and block 1332, the return electrode-heat sink is removed from contact with skin over the four electrodes. The program continues as represented at line 1334 to the query posed at block 1336. In this regard, a determination is made, as to whether the thermal wand is to be used at a second location within this heating channel. In the event that it is to be so used, then as represented at line 1338 and block 1340, the thermal wand is withdrawn to the second position within the instant heating channel. Then, as represented at line 1342 the procedure reverts to node C, which is associated with line 1306 extending to line 1244.

Returning to block 1336, where the thermal wand is not to be used at a second location within the given heating channel, then as represented at line 1344 and block 1346 the thermal wand is removed. Next, as represented at line 1348 and block 1350, where required, the practitioner may form a radially spaced heating channel extending from the present entrance incision. Accordingly, as represented at line 1352 and block 1354, as required, the therapy is reactivated. At the completion of the therapy, as represented at line 1356 and block 1358, the thermal wand is removed and, as represented at line 1360 and block 1362, all entrance incisions are repaired whereupon, as represented at line 1364 and block 1366, a clearance interval ensues. For a vascular anomaly, this clearance interval generally will be measured in terms of weeks. Following the clearance interval, the patient will have reappeared and as indicated by line 1368 and block 1370, a determination is made as to whether there is any remaining vascular anomaly. In the event of an affirmative determination with respect to the query posed at block 1370, then as represented at line 1372 the program reverts to node D. Node D reappears in conjunction with line 1374, extending to line 1062. Returning to block 1370, where no substantial vascular anomaly remains, then as represented at line 1376 and block 1378, therapy is completed.

Since certain changes may be made to the above apparatus, method and system without departing from the scope of the disclosure herein involved, it is intended that all matter contained in the above description or shown in the accompanying drawing shall be interpreted as illustrative and not in a limiting sense.

1. The method for effecting a heating of tissue within the region of the dermis of skin, comprising the steps:
(a) determining a skin region for treatment;
(b) providing one or more implants each having one or more R.F. excitable active electrodes, each electrode being associated in thermal exchange relationship with a temperature sensor having a sensor output;
(c) providing one or more combined return electrode and heat sink assemblages each having one or more contact surfaces;
(d) determining one or more heating channel locations along said skin region, each extending from an entrance location;
(e) locating each implant along a heating channel generally at the interface between dermis and next adjacent subcutaneous tissue wherein said one or more active electrodes are contactable with dermis;
(f) selecting a setpoint temperature threshold level for said one or more electrodes;
(g) positioning said one or more combined return electrode and heat sink assemblages contact surfaces over and in substantial alignment with said active electrodes and against epidermis in heat and electrical transfer relationship; and
(h) effecting a radiofrequency power energization at said one or more active electrodes for an energization interval wherein said energization is carried out for at least a portion of said energization interval during power-on intervals spaced apart in time by power-off intervals to reach and substantially maintain said setpoint temperature threshold level while avoiding thermal trauma to said epidermis.

2. The method of claim 1 in which:
step (g) includes the step of locating an electrically and thermally conductive material between said contact surface and said epidermis.

3. The method of claim 2 in which:
said electrically and thermally conductive material is isotonic saline solution.

4. The method of claim 1 in which:
step (d) generally determines said one or more heating channel locations along relaxed intrinsic skin tension lines.

5. The method of claim 1 in which:
step (b) provides said implants as having a light emitting diode aligned with and distal to said one or more electrodes and another light emitting diode aligned with and proximal to said one or more electrodes, said one and another light emitting diodes being energizable to emit light in a region of the spectrum effective to penetrate tissue; and
step (g) effects said substantial alignment of said contact surfaces while said light emitting diodes are energized.

6. The method of claim 5 in which:
step (b) provides said light emitting diodes as being energizable to emit in the red region of the spectrum.

7. The method of claim 1 in which:
step (e) initially locates an implant to position said one or more electrodes at an initial location along a heating channel, whereupon steps (g) and (h) are carried out, whereupon the implant is relocated within the heating channel to an extent positioning said one or more electrodes at a second untreated position whereupon steps (g) and (h) are repeated.

8. The method of claim 7 in which:
said initial position is forwardly along said heating channel; and
said second position is derived by pulling said implant toward its associated entrance location.

9. The method of claim 1 in which:
step (c) provides said return electrode and heat sink assemblage as an articulate combination of aluminum blocks manipulable to substantially conform the contact surface to the profile of skin within said region.

10. The method of claim 5 in which:
step (c) provides said return electrode and heat sink assemblage as one or more aluminum blocks supporting two photo-detectors centrally disposed adjacent a said contact surface and spaced apart a distance matching the space between said two light emitting diodes and having detector outputs responsive to light emitting diode emissions.

11. The method of claim 1 in which:
step (h) effects said radiofrequency energization during power-on intervals spaced apart in time by power-off intervals within one or more time stages of duration effective to avoid thermal trauma to tissue underlying dermis.

12. The method of claim 11 in which:
step (h) is carried out with said time stages being spaced apart by cool-down intervals of duration effective to cool tissue underlying dermis to non-traumatic temperature level.

13. The method of claim 1 in which:
step (c) provides said combined return electrode and heat sink as having two mutually electrically isolated return electrode components; and
step (g) includes the step of carrying out a patient circuit safety monitoring procedure.

14. Implant apparatus for effecting a controlled heating of tissue at the region of the dermis from a heating channel location generally at the interface of dermis and next adjacent subcutaneous tissue, comprising:
a thermally insulative generally flat support having a support surface and an oppositely disposed insulative surface having a lengthwise dimension extending along an implant axis between leading and trailing ends and a widthwise dimension between longitudinal edges extending along an insertion length;
an electrode circuit supported from said support surface having one or more electrodes of electrode length and formed of two or more electrically interconnected electrode segments having electrode width defining edges and energizable from a radiofrequency source to generate heat within tissue at the region of the dermis; and
a lead assemblage extending in electrical communication from each electrode to a lead contact region adjacent said support trailing end.

15. The implant apparatus of claim 14 in which:
each electrode width defining edge is spaced from an adjacent longitudinal edge a distance of from about 0.030 inch to about 0.100 inch.

16. The implant apparatus of claim 14 in which:
said electrode segments are present in a longitudinally spaced apart sequence, each arranged transversely to said implant axis.

17. The implant apparatus of claim 14 in which:
said electrode segments are present in a spaced apart sequence each arranged in parallel with said implant axis.

18. The implant apparatus of claim 14 in which:
said lead assemblage is electrically insulated at least where contactable with tissue.

19. The implant apparatus of claim 14 further comprising:
a light emitting diode aligned with and distal to said one or more electrodes and another light emitting diode aligned with and proximal to said one or more electrodes, said light emitting diodes being energizable to emit light in a region of the spectrum effective to penetrate tissue.

20. The implant apparatus of claim 19 in which:
said light emitting diodes are energizable to emit in the red region of the spectrum.

21. The implant apparatus of claim 14 in which:
said electrode circuit is located upon an electrically insulative electrode support substrate having an outer surface and an oppositely disposed inner surface supported from said support surface and extending to said trailing end.

22. The implant apparatus of claim 19 further comprising”
one or more electrically energizable resistor segments with a resistor lead assemblage extending therefrom located upon the outer surface of an electrically insulative resistor support substrate having an inner surface supported at said flat support surface and extending over said trailing end to expose a portion of said resistor lead assemblage at said insulative surface generally opposite said lead contact region; and
said electrode substrate inner surface is supported over said resistor support outer surface.

23. The implant apparatus of claim 22 in which:
said resistor lead assemblage is configured to provide four-point electrical connection with each resistor segment.

24. The implant apparatus of claim 14 in which:
said support leading end is slanted forwardly to an extent effective to provide a mechanical bias toward dermis when the implant is injected into said interface.

25. The implant apparatus of claim 14 in which:
the lengthwise dimension of said support is about 180 millimeters; and
said electrode circuit is configured with four electrodes mutually spaced apart about one millimeter and having a lengthwise extent of about ten millimeters and extending along said implant axis from a location generally adjacent said leading end.

26. The implant apparatus of claim 25 in which:
said electrodes generally exhibit a widthwise extent of about 3.7 millimeters.

27. Return electrode and heat sink apparatus for employment with radiofrequency driven implant apparatus supporting a sequence of active electrodes and locatable within an epidermis region of given contour generally at the interface between dermis and the next adjacent subcutaneous tissue, comprising:
a plurality of generally rectangular blocks formed of thermally and electrically conductive material, having oppositely disposed slide surfaces extending between an outer surface and a receiving surface, said blocks being joined in freely abutting relationship at said slide surfaces by a tensioned retainer located adjacent said outer surface and extending between oppositely disposed end blocks to form a sequence of mutually abutting blocks;
a thin flexible thermally and electrically conductive shim extending across and fixed to and electrically interconnecting the sequence of blocks while permitting slideable movement thereof to generally accommodate said given contour; and
a terminal assembly configured to electrically couple said sequence of blocks as a return electrode with a source of R.F. energy.

28. The return electrode and heat sink apparatus of claim 27 in which:
said blocks are formed of aluminum.

29. The return electrode and heat sink apparatus of claim 27 in which:
said sequence of blocks define a thermal mass effective to avoid thermal trauma to epidermis.

30. The return electrode and heat sink apparatus of claim 27 in which:
said implant apparatus is configured with two light emitting diodes, one being located adjacent the proximate end of said sequence of active electrodes and the other being located adjacent the distal end of said sequence of active electrodes, said light emitting diodes being energizable to emit in a portion of the light spectrum effective to penetrate tissue; and
two of said blocks are configured supporting two photodetectors located centrally of said slide surfaces and spaced apart a distance matching the space between said two light emitting diodes and having detector outputs when responding to the emissions thereof.

31. The return electrode and heat sink apparatus of claim 27 in which:
said shim is formed of a copper material bonded with said receiving surfaces with a thermally and electrically conductive adhesive.

32. The return electrode and heat sink apparatus of claim 27 in which:
said tensioned retainer is a coil spring extending through aligned openings within said blocks between said end blocks.

33. The return electrode and heat sink apparatus of claim 27 in which:
said sequence of abutting blocks is configured to define two mutually electrically isolated block sequences; and
said terminal assembly is effective additionally for performance in the carrying out of a patient circuit safety monitor operation.

34. The return electrode and heat sink apparatus of claim 27 further comprising:
a heat sink temperature sensor generally responsive to temperature at said skin surface to provide a heat sink temperature output.

35. The return electrode and heat sink apparatus of claim 27 in which:
said sequence of mutually abutting blocks has a length of about 3.75 inches, a width of about 0.75 inch and a height of about 1.5 inches.

36. A system for effecting a heating of tissue within the region of the dermis of skin, comprising:
one or more implants each having one or more active electrodes excitable from an R.F. source and having an electrode width defining edge, each electrode being associated in thermal exchange relationship with a temperature sensor having a sensor output, each implant being configured with a thermally insulative flat support having a widthwise dimension between longitudinal edge, configured for insertion within a heating channel located at the interface between dermis and next adjacent subcutaneous tissue wherein said one or more active electrodes are contactable with dermis;
a combined return electrode and heat sink formed of a thermally and electrically conductive metal material having a contact surface engagable with skin over said one or more active electrodes and having a terminal assembly connectible as a return with said R.F. source; and
a controller including said R.F. source connectible in energizing relationship with said active one or more electrodes and in return relationship with said terminal assembly, said controller being responsive to the sensor outputs and to an elected setpoint temperature threshold level when actuated to effect the R.F. energization at said one or more electrodes for an energization interval wherein such energization is carried out for at least a portion of said energization interval during power-on intervals spaced apart in time by power-off intervals to attain said setpoint temperature threshold level.

37. The system of claim 36 further comprising:
an electrically and thermally conductive material locatable between the combined return electrode and heat sink contact surface and the surface of skin over said electrodes.

38. The system of claim 37 in which:
said electrically and thermally conductive material is isotonic saline solution.

39. The system of claim 36 in which:
said controller is responsive to an electrode high limit temperature above said setpoint threshold temperature to terminate said electrode energization.

40. The system of claim 39 in which:
said controller is responsive, subsequent to an electrode reaching said high limit temperature to lower the voltage associated with an initial power-on interval.

41. The system of claim 36 in which:
said controller, when initially actuated effects the R.F. energization at said one or more electrodes for a ramp period applying increasing electrode drive voltage until said setpoint threshold temperature is reached.

42. The system of claim 41 in which:
said controller is initially responsive, subsequent to said ramp period to effect said R.F. energization during power-on intervals spaced apart in time by power-off intervals to define a sequence of uniform energization pulses within an initial time stage of duration effective to avoid thermal trauma to tissue underlying dermis.

43. The system of claim 42 in which:
said controller is responsive to effect a power-off condition during a cool-down period subsequent to said initial time stage until said one or more active electrode temperature reaches a lower limit temperature, where upon the initial power-on and power-off defined pulse of a next subsequent time stage is effected.

44. The system of claim 43 in which:
said controller maintains the initial power-on defined pulse of the next subsequent time stage until the temperature of said one or more active electrodes reaches the setpoint threshold temperature whereupon the controller carries out power-off and power-on conditions defining a sequence of uniform power pulses for the remainder of said next subsequent time stage.

45. The system of claim 44 in which:
said lower limit temperature is selected to effect a lowering of the temperature of said tissue underlying dermis to substantially return to normal body temperature.

46. The system of claim 45 in which:
said controller is responsive to continue the carrying out of time stages spaced apart by cool-down period until therapy is completed

47. The system of claim 42 in which:
said controller is responsive to effect a power-off condition during a cool-down period of predetermined extent effective to avoid thermal trauma to tissue underlying dermis, whereupon the initial power-on and power-off defined pulse of a next subsequent time stage is effected,

48. The system of claim 36 in which:
said over-temperature warning cue is an audible word-based cue.

49. The system of claim 36 in which:
said combined return electrode and heat sink includes a heat sink temperature sensor generally responsive to temperature at said contact surface to provide a heat sink temperature output; and
said controller is responsive to said heat sink temperature output representing a temperature at or above a contact surface temperature limit to promulgate a humanly perceptible heat sink over-temperature cue.

50. The system of claim 49 in which:
said heat sink over-temperature cue is an audible word-based cue.

51. The system of claim 36 in which:
said controller is responsive at any termination of said energization interval to establish a cool-down interval wherein said contact surface is retained against skin surface above said one or more active electrodes.

52. The system of claim 51 in which:
said controller is responsive at the termination of said cool-down interval to provide a cool-down completed humanly perceptible cue.

53. The system of claim 52 in which:
said cool-down completed cue is an audible word-based cue.

54. The system of claim 51 in which:
said controller is responsive at the commencement of said cool-down interval to provide a cool-down commencement humanly perceptible cue.

55. The system of claim 36 in which:
said controller is responsive at any termination of said energization interval to establish a post therapy cool-down interval wherein said contact surface is retained against skin surface above said one or more active electrodes.

56. The system of claim 51 in which:
said controller is responsive at the termination of said post therapy cool-down interval to provide a cool-down completed humanly perceptible cue.

57. The system of claim 52 in which:
said cool-down completed cue is an audible word-based cue.

58. The system of claim 51 in which:
said controller is responsive at the commencement of said post therapy cool-down interval to provide a cool-down commencement humanly perceptible cue.

59. The system of claim 54 in which:
said commencement of post therapy cool-down interval cue is an audible word-based cue.

60. The system of claim 36 in which:
said return electrode and heat sink is configured to define two mutually electrically isolated contact surfaces;
said terminal assembly is effective additionally for performance in the carrying out of a patient circuit safety monitor operation; and
said controller is responsive upon said actuation to effect the carrying out of a patient circuit safety monitor operation.

61. The system of claim 36 in which:
said one or more implants are configured with a first light emitting diode aligned with and distal to said one or more electrodes and a second light emitting diode aligned with and proximal to said one or more electrodes, said first and second light emitting diodes being engageable from said controller to emit light in a region of the spectrum effective to penetrate tissue; and
said controller is configured to energize said first and second light emitting diodes when they are electrically coupled therewith.

62. The system of claim 60 in which:
said first and second light emitting diodes emit in the red region of the spectrum.

63. The system of claim 62 in which:
said combined return electrode and heat sink is configured supporting a first photodetector located for deriving a first detector output in response to emission from said first light emitting diode and a second photodetector located for deriving a second detector output in response to emission from said second light emitting diode; and
said controller is respondive in the absence of either said first or second detector outputs to negate the energization of said one or more electrodes.

64. The system of claim 63 in which:
said controller is responsive to the absence of either said first or second detector outputs to promulgate a humanly perceptible misalignment cue.

65. The system of claim 64 in which:
said misalignment cue is an audible word-based cue.

66. The system of claim 36 in which:
each electrode width defining edge is spaced from an adjacent longitudinal edge a distance effective to avoid the presence of aberrational current flow toward said next adjacent subcutaneous tissue.

67. The system of claim 66 in which:
each electrode width defining edge is spaced from an adjacent longitudinal edge a distance of from about 0.030 to about 0.100 inch.

68. Return electrode and heat sink apparatus for employment with radiofrequency driven implant apparatus supporting a sequence of active electrodes locatable within an epidermis region generally at the interface between dermis and the next adjacent subcutaneous tissue, and configured with two light emitting diodes, one being located adjacent the distal end of said sequence of active electrodes and the other being located adjacent the proximal end of said sequence of active electrodes, said light emitting diodes being energizable to emit in a portion of the light spectrum effective to penetrate tissue, comprising:
at least one generally rectangular block formed of thermally and electrically conductive material, having a contact surface dimensioned to define a return electrode effective to return radiofrequency current generated from said electrode sequence and supporting two photo-detectors located centrally of said contact surface and spaced apart a distance matching the space between said two light emitting diodes and having detector outputs when responding to the emissions thereof; and
a terminal assembly associated with said block effective to define said return electrode and enable said photo-detectors.

69. The return electrode of claim 68 in which:
said at least one block is formed of aluminum.

70. The return electrode of claim 68 in which:
said at least one block defines a thermal mass effective to avoid thermal trauma to epidermis.

71. The return electrode of claim 68 in which:
said at least one block has a length of about 3.75 inches, a width of about 0.75 inch and a height of about 1.5 inches.
